(19) United States
(12) Patent Application Publication
(10) Pub. No.: US 2021/0115044 A1
(43) Pub. Date: Apr. 22, 2021

(54) TRICYCLIC HETEROCYCLE COMPOUNDS USEFUL AS HIV INTEGRASE INHIBITORS

(71) Applicant: Merck Sharp & Dohme Corp., Rahway, NJ (US)

(72) Inventors: Tao Yu, Edison, NJ (US); James M. Apgar, Highland Park, NJ (US); Alan Whitehead, Doylestown, PA (US); Yonglian Zhang, East Brunswick, NJ (US); Zhiyong Hu, Livingston, NJ (US); Valerie W. Shurtleff, Philadelphia, PA (US); John A. McCauley, Maple Glen, PA (US); Izzat T. Raheem, Doylestown, PA (US)

(73) Assignee: Merck Sharp & Dohme Corp., Rahway, NJ (US)

(21) Appl. No.: 17/048,811

(22) PCT Filed: Apr. 22, 2019
(86) PCT No.: PCT/US19/28432

§ 371 Date: Oct. 19, 2020

Related U.S. Application Data
(60) Provisional application No. 62/663,377, filed on Apr. 27, 2018.

(51) Int. Cl. C07D 471/16; A61K 31/4985; A61P 31/18; A61K 31/52; A61K 31/435; A61K 31/5383; A61K 31/506; A61K 31/513; A61K 31/685; A61K 31/427

(52) U.S. Cl. CPC C07D 471/16; A61K 31/4985; A61P 31/18; A61K 31/52; A61K 31/427; A61K 31/5383; A61K 31/506; A61K 31/513; A61K 31/685; A61K 31/435

(57) ABSTRACT

The present invention relates to Tricyclic Heterocycle Compounds of Formula (I): (I) and pharmaceutically acceptable salts or prodrug thereof, wherein R¹, R², R³, R⁴, R⁵, R⁶ and n are as defined herein. The present invention also relates to compositions comprising at least one Tricyclic Heterocycle Compound, and methods of using the Tricyclic Heterocycle Compounds for treating or preventing HIV infection in a subject.

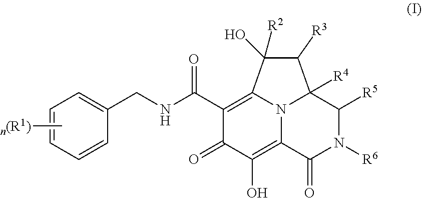

### FIELD OF THE INVENTION

The present invention relates to Tricyclic Heterocycle Compounds, compositions comprising at least one Tricyclic Heterocycle Compound, and methods of using the Tricyclic Heterocycle Compounds for treating or preventing HIV infection in a subject.

### BACKGROUND OF THE INVENTION

A retrovirus designated human immunodeficiency virus (HIV), particularly the strains known as HIV type-1 (HIV-1) virus and type-2 (HIV-2) virus, is the etiological agent of the complex disease that includes progressive destruction of the immune system (acquired immune deficiency syndrome; AIDS) and degeneration of the central and peripheral nervous system. A common feature of retrovirus replication is the insertion by virally-encoded integrase of +proviral DNA into the host cell genome, a required step in HIV replication in human T-lymphoid and monocytoid cells. Integration is believed to be mediated by integrase in three steps: assembly of a stable nucleoprotein complex with viral DNA sequences; cleavage of two nucleotides from the 3′ termini of the linear proviral DNA, and covalent joining of the recessed 3′ OH termini of the proviral DNA at a staggered cut made at the host target site. The fourth step in the process, repair synthesis of the resultant gap, may be accomplished by cellular enzymes.

Nucleotide sequencing of HIV shows the presence of a pol gene in one open reading frame [Ratner, L. et al., Nature, 313, 277(1985)]. Amino acid sequence homology provides evidence that the pol sequence encodes reverse transcriptase, integrase and an HIV protease [Toh, H. et al., EMBO J. 4, 1267 (1985); Power, M. D. et al., Science, 231, 1567 (1986); Pearl, L. H. et al., Nature, 329, 351 (1987)]. All three enzymes have been shown to be essential for the replication of HIV.

It is known that some antiviral compounds which act as inhibitors of HIV replication are effective agents in the treatment of AIDS and similar diseases, including reverse transcriptase inhibitors such as azidothymidine (AZT) and efavirenz and protease inhibitors such as indinavir and nelfinavir. The compounds of this invention are inhibitors of HIV integrase and inhibitors of HIV replication.

### SUMMARY OF THE INVENTION

In one aspect, the present invention provides Compounds of Formula (I):

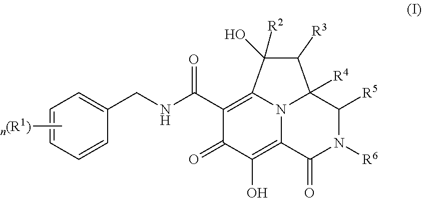

or a pharmaceutically acceptable salt thereof,
wherein:

each occurrence of R¹ is independently halo, hydroxyl, C₁₋₆ alkyl and —O—(C₁-C₆ alkyl);

R² is hydrogen, methyl or ethyl;

R³ is hydrogen, methyl or ethyl;

R⁴ is C₁₋₆ alkyl or (C₁₋₆ alkyl)OR⁷;

R⁵ is hydrogen, C₁₋₆ alkyl or (C₁₋₆ alkyl)OR⁷;

R⁶ is hydrogen, C₁₋₆ alkyl or (C₁₋₆ alkyl)OR⁷;

R⁷ is hydrogen or C₁₋₆ alkyl, which is optionally substituted with one to three halo;

n is an integer between one and three.

The Compounds of Formula (I) (also referred to herein as the “Tricyclic Heterocycle Compounds”) and pharmaceutically acceptable salts or prodrugs thereof may be useful, for example, for inhibiting HIV viral replication or replicon activity, or for treating or preventing HIV infection in a subject. Without being bound by any specific theory, it is believed that the Tricyclic Heterocycle Compounds inhibit HIV viral replication by inhibiting HIV Integrase.

Accordingly, the present invention provides methods for treating or preventing HIV infection in a subject, comprising administering to the subject an effective amount of at least one Tricyclic Heterocycle Compound.

The details of the invention are set forth in the accompanying detailed description below.

Although any methods and materials similar to those described herein may be used in the practice or testing of the present invention, illustrative methods and materials are now described. Other embodiments, aspects and features of the present invention are either further described in or will be apparent from the ensuing description, examples and appended claims.

### DETAILED DESCRIPTION OF THE INVENTION

The present invention includes Tricyclic Heterocycle Compounds, compositions comprising at least one Tricyclic Heterocycle Compound, and methods of using the Tricyclic Heterocycle Compounds for treating or preventing HIV infection in a subject.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “-O-alkyl,” etc.

As used herein, and throughout this disclosure, the following terms, unless otherwise indicated, shall be understood to have the following meanings:

A “subject” is a human or non-human mammal. In one embodiment, a subject is a human. In another embodiment, a subject is a primate. In another embodiment, a subject is a monkey. In another embodiment, a subject is a chimpanzee. In still another embodiment, a subject is a rhesus monkey.

The term “effective amount” as used herein, refers to an amount of Tricyclic Heterocycle Compound and/or an additional therapeutic agent, or a composition thereof that is effective in inhibiting HIV replication and in producing the desired therapeutic, ameliorative, inhibitory or preventative effect when administered to a subject suffering from HIV infection or AIDS. In the combination therapies of the present invention, an effective amount can refer to each individual agent or to the combination as a whole, wherein the amounts of all agents administered are together effective, but wherein the component agent of the combination may not be present individually in an effective amount.

The terms “treating” or “treatment” as used herein with respect to an HIV viral infection or AIDS, includes inhibiting the severity of HIV infection or AIDS a disease, i.e., arresting or reducing the development of the HIV infection or AIDS a disease or its clinical symptoms; or relieving the HIV infection or AIDS a disease, i.e., causing regression of the severity of HIV infection or AIDS a disease or its clinical symptoms.

The terms “preventing,” or “prohylaxis,” as used herein with respect to an HIV viral infection or AIDS, refers to reducing the likelihood or severity of HIV infection or AIDS.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. In one embodiment, an alkyl group is linear. In another embodiment, an alkyl group is branched. Unless otherwise indicated, an alkyl group is unsubstituted.

The term “halo,” as used herein, means —F, —Cl, —Br or —I.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃. The term “C₁-C₆ haloalkyl” refers to a haloalkyl group having from 1 to 6 carbon atoms.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any substituent or variable (e.g., R² and R³) occurs more than one time in any constituent or in Formula (I), its definition on each occurrence is independent of its definition at every other occurrence, unless otherwise indicated.

As used herein, the term “composition” is intended to encompass a product comprising the specified ingredients in the specified amounts, as well as any product which results from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a Tricyclic Heterocycle Compound or a pharmaceutically acceptable salt of the compound. The transformation may occur by various mechanisms (e.g., by metabolic or chemical processes), such as, for example, through hydrolysis in blood. For example, if a Tricyclic Heterocycle Compound or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₅)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as 0-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a Tricyclic Heterocycle Compound contains an alcohol functional group, a prodrug can be formed by the replacement of one or more of the hydrogen atoms of the alcohol groups with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, U-amino(C₁-C₄)alkylene-aryl, arylacyl and α-aminoacyl, or α-aminoacyl-α-aminoacyl, where each α-aminoacyl group is independently selected from the naturally occurring L-amino acids, or glycosyl (the radical resulting from the removal of a hydroxyl group of the hemiacetal form of a carbohydrate).

If a Tricyclic Heterocycle Compound incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N- or di-N,N—(C₁-C₆)alkylaminoalkyl; —C(Y⁴)Y wherein Y⁴ is H or methyl and Y⁵ is mono-N- or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino); (2) sulfonate esters, such as alkyl- or aralkylsulfonyl (for example, methanesulfonyl); (3) amino acid esters, including those corresponding to both natural and non-natural amino acids (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvates, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The Tricyclic Heterocycle Compounds can form salts which are also within the scope of this invention. Reference to a Tricyclic Heterocycle Compound herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Tricyclic Heterocycle Compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a Tricyclic Heterocycle Compound with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), arylalkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Tricyclic Heterocycle Compounds may be atropisomers (e.g., substituted biaryls) and are considered as part of this invention. Enantiomers can also be directly separated using chiral chromatographic techniques.

It is also possible that the Tricyclic Heterocycle Compounds may exist in different tautomeric forms, and all such forms are embraced within the scope of the invention. For example, all keto-enol and imine-enamine forms of the compounds are included in the invention.

Unless otherwise indicated, all stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a Tricyclic Heterocycle Compound incorporates a double bond or a fused ring, both the cis- and trans-forms, as well as mixtures, are embraced within the scope of the invention.

When a substituent on a chiral carbon atom is depicted without specific stereochemistry (by using a straight line bond to a chiral center), it is to be understood that both the alpha and beta configurations of said substituent group are to be considered part of the present invention. For example, the compound of the present invention, which is drawn as follows:

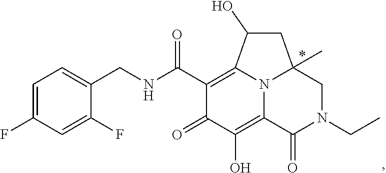 [SMILES: CCN1CC2(C)CC(O)c3c(C(=O)NCc4ccc(F)cc4F)c(=O)c(O)c(n32)C1=O]

is understood to encompass both stereoisomers at the indicated chiral center, the structures of which are as follows:

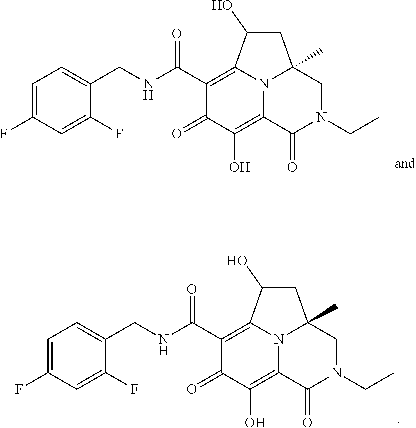 [SMILES: CCN1C[C@]2(C)CC(O)c3c(C(=O)NCc4ccc(F)cc4F)c(=O)c(O)c(n32)C1=O ; CCN1C[C@@]2(C)CC(O)c3c(C(=O)NCc4ccc(F)cc4F)c(=O)c(O)c(n32)C1=O]

In the Examples section below, compounds of the present invention that have been purified as individual stereoisomers are sometimes depicted in non-stereospecific form but identified using one or more of the terms: “diastereomer 1,” “diastereomer 2,” “isomer 1,” “isomer 2,” “enantiomer A” and “enantiomer B.” In this instance, the absolute stereochemistry of each isolated diastereomer and enantiomeric center has not been determined and the terms used above are used to represent each individual purified stereochemically pure compound.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may provide certain therapeutic advantages, such as increasing in vivo half-life or reducing dosage requirements, or may provide a compound useful as a standard for characterization of biological samples. Isotopically-enriched Compounds of Formula (I) can be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

The Tricyclic Heterocycle Compounds may be useful in human and veterinary medicine for treating or preventing HIV infection in a subject. In one embodiment, the Tricyclic Heterocycle Compounds can be inhibitors of HIV viral replication. In a specific embodiment, the Tricyclic Heterocycle Compounds are inhibitors of HIV-1. Accordingly, the Tricyclic Heterocycle Compounds may be useful for treating HIV infections and AIDS. In accordance with the invention, the Tricyclic Heterocycle Compounds can be administered to a subject in need of treatment or prevention of HIV infection.

Accordingly, in one embodiment, the invention provides methods for treating HIV infection in a subject comprising administering to the subject an effective amount of at least one Tricyclic Heterocycle Compound or a pharmaceutically acceptable salt thereof. In a specific embodiment, the present invention provides methods for treating AIDS in a subject comprising administering to the subject an effective amount of at least one Tricyclic Heterocycle Compound or a pharmaceutically acceptable salt thereof.

### The Compounds of Formula (I)

The present invention provides Tricyclic Heterocycle Compounds of Formula (I):

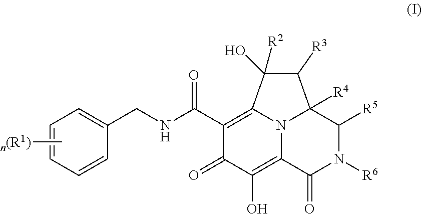

and pharmaceutically acceptable salts thereof, wherein:
wherein:
each occurrence of R¹ is independently halo, hydroxyl, C₁₋₆ alkyl and —O—(C₁-C₆ alkyl);

R² is hydrogen, methyl or ethyl;

R³ is hydrogen, methyl or ethyl;

R⁴ is C₁₋₆ alkyl or (C₁₋₆ alkyl)OR⁷;

R⁵ is hydrogen, C₁₋₆ alkyl or (C₁₋₆ alkyl)OR⁷;

R⁶ is hydrogen, C₁₋₆ alkyl or (C₁₋₆ alkyl)OR⁷;

R⁷ is hydrogen or C₁₋₆ alkyl, which is optionally substituted with one to three halo;

n is an integer between one and three.

In an embodiment of the invention, R¹ is halo. In a class of the embodiment, R¹ is fluoro. In a class of the embodiment, R is chloro.

In an embodiment of the invention, R² is hydrogen or methyl. In a class of the invention, R² is hydrogen. In another class of the invention, R² is methyl.

In an embodiment of the invention, R³ is hydrogen or methyl. In a class of the invention, R³ is hydrogen. In another class of the invention, R³ is methyl.

In an embodiment of the invention, R⁴ is methyl, ethyl, CH₂OCH₃, CH₂CH₂OCH₃ or CH₂CH₂OCHF₂. In a class of the invention, R⁴ is methyl or ethyl. In another class of the invention, R⁴ is methyl. In another class of the invention, R⁴ is ethyl. In another class of the invention, R⁴ is CH₂OCH₃. In another class of the invention, R⁴ is CH₂CH₂OCH₃. In another class of the invention, R⁴ is CH₂CH₂OCHF₂.

In an embodiment of the invention, R⁵ is C₁₋₆ alkyl. In another embodiment of the invention, R⁵ is hydrogen or methyl. In a class of the invention, R⁵ is methyl. In another class of the invention, R⁵ is hydrogen.

In an embodiment of the invention, R⁶ is C₁₋₆ alkyl. In a class of the invention, R⁶ is methyl or ethyl. In another class of the invention, R⁶ is methyl. In another class of the invention, R⁶ is ethyl. In another embodiment of the invention, R⁶ is hydrogen.

In an embodiment of the invention, n is one. In another embodiment of the invention, n is two. In another embodiment of the invention, n is three.

In another embodiment, the Compounds of Formula (I) are in substantially purified form.

It is to be understood that any of the aforementioned embodiments may be combined with one or more separate embodiments.

Other embodiments of the present invention include the following:

(a) A pharmaceutical composition comprising an effective amount of a Compound of Formula (I), and a pharmaceutically acceptable carrier.

(b) The pharmaceutical composition of (a), further comprising a second therapeutic agent selected from the group consisting of HIV antiviral agents, immunomodulators, and anti-infective agents.

(c) The pharmaceutical composition of (b), wherein the HIV antiviral agent is an antiviral selected from the group consisting of HIV protease inhibitors and HIV NNRTI inhibitors.

(d) A pharmaceutical combination that is (i) a Compound of Formula (I) and (ii) a second therapeutic agent selected from the group consisting of HIV antiviral agents, immunomodulators, and anti-infective agents; wherein the Compound of Formula (I) and the second therapeutic agent are each employed in an amount that renders the combination effective for inhibiting HIV replication, or for treating HIV infection and/or reducing the likelihood or severity of symptoms of HIV infection.

(e) The combination of (d), wherein the HIV antiviral agent is an antiviral selected from the group consisting of HIV protease inhibitors and HIV NNRTI inhibitors.

(f) A method of inhibiting HIV replication in a subject in need thereof which comprises administering to the subject an effective amount of a Compound of Formula (I).

(g) A method of treating HIV infection and/or reducing the likelihood or severity of symptoms of HIV infection in a subject in need thereof which comprises administering to the subject an effective amount of a Compound of Formula (I).

(h) The method of (g), wherein the Compound of Formula (I) is administered in combination with an effective amount of at least one second therapeutic agent selected from the group consisting of HIV antiviral agents, immunomodulators, and anti-infective agents.

(i) The method of (h), wherein the HIV antiviral agent is an antiviral selected from the group consisting of HIV protease inhibitors and HIV NNRTI inhibitors.

(j) A method of inhibiting HIV replication in a subject in need thereof which comprises administering to the subject the pharmaceutical composition of (a), (b) or (c) or the combination of (d) or (e).

(k) A method of treating HIV infection and/or reducing the likelihood or severity of symptoms of HIV infection in a subject in need thereof which comprises administering to the subject the pharmaceutical composition of (a), (b) or (c) or the combination of (d) or (e).

Additional embodiments of the present invention include the following:

(l) A pharmaceutical composition comprising an effective amount of a pharmaceutically acceptable salt of a Compound of Formula (I), and a pharmaceutically acceptable carrier.

(m) The pharmaceutical composition of (l), further comprising a second therapeutic agent selected from the group consisting of HIV antiviral agents, immunomodulators, and anti-infective agents.

(n) The pharmaceutical composition of (m), wherein the HIV antiviral agent is an antiviral selected from the group consisting of HIV protease inhibitors and HIV NNRTI inhibitors.

(o) A pharmaceutical combination that is (i) a pharmaceutically acceptable salt of a Compound of Formula (I) and (ii) a second therapeutic agent selected from the group consisting of HIV antiviral agents, immunomodulators, and anti-infective agents; wherein the pharmaceutically acceptable salt of the Compound of Formula (I) and the second therapeutic agent are each employed in an amount that renders the combination effective for inhibiting HIV replication, or for treating HIV infection and/or reducing the likelihood or severity of symptoms of HIV infection.

(p) The combination of (o), wherein the HIV antiviral agent is an antiviral selected from the group consisting of HIV protease inhibitors and HIV NNRTI inhibitors.

(q) A method of inhibiting HIV replication in a subject in need thereof which comprises administering to the subject an effective amount of a pharmaceutically acceptable salt of a Compound of Formula (I).

(r) A method of treating HIV infection and/or reducing the likelihood or severity of symptoms of HIV infection in a subject in need thereof which comprises administering to the subject an effective amount of a pharmaceutically acceptable salt of a Compound of Formula (I).

(s) The method of (r), wherein the pharmaceutically acceptable salt of the Compound of Formula (I) is administered in combination with an effective amount of at least one second therapeutic agent selected from the group consisting of HIV antiviral agents, immunomodulators, and anti-infective agents.

(t) The method of (s), wherein the HIV antiviral agent is an antiviral selected from the group consisting of HIV protease inhibitors and HIV NS5B polymerase inhibitors.

(u) A method of inhibiting HIV replication in a subject in need thereof which comprises administering to the subject the pharmaceutical composition of (1), (m) or (n) or the combination of (o) or (p).

(v) A method of treating HIV infection and/or reducing the likelihood or severity of symptoms of HIV infection in a subject in need thereof which comprises administering to the subject the pharmaceutical composition of (1), (m) or (n) or the combination of (o) or (p).

Further embodiments of the present invention include the following:

(w) A pharmaceutical composition comprising an effective amount of a Compound of Formula (I) or a pharmaceutically acceptable salt thereof, and a pharmaceutically acceptable carrier.

(x) The pharmaceutical composition of (w), further comprising a second therapeutic agent selected from the group consisting of HIV antiviral agents, immunomodulators, and anti-infective agents.

(y) The pharmaceutical composition of (x), wherein the HIV antiviral agent is an antiviral selected from the group consisting of HIV protease inhibitors and HIV NNRTI inhibitors.

(z) A pharmaceutical combination that is (i) a Compound of Formula (I) and (ii) or a pharmaceutically acceptable salt thereof, a second therapeutic agent selected from the group consisting of HIV antiviral agents, immunomodulators, and anti-infective agents; wherein the Compound of Formula (I) and the second therapeutic agent are each employed in an amount that renders the combination effective for inhibiting HIV replication, or for treating HIV infection and/or reducing the likelihood or severity of symptoms of HIV infection.

(aa) The combination of (z), wherein the HIV antiviral agent is an antiviral selected from the group consisting of HIV protease inhibitors and HIV NNRTI inhibitors.

(bb) A method of inhibiting HIV replication in a subject in need thereof which comprises administering to the subject an effective amount of a Compound of Formula (I) or a pharmaceutically acceptable salt thereof.

(cc) A method of treating HIV infection and/or reducing the likelihood or severity of symptoms of HIV infection in a subject in need thereof which comprises administering to the subject an effective amount of a Compound of Formula (I) or a pharmaceutically acceptable salt thereof.

(dd) The method of (cc), wherein the Compound of Formula (I) or pharmaceutically acceptable salt thereof, is administered in combination with an effective amount of at least one second therapeutic agent selected from the group consisting of HIV antiviral agents, immunomodulators, and anti-infective agents.

(ee) The method of (dd), wherein the HIV antiviral agent is an antiviral selected from the group consisting of HIV protease inhibitors and HIV NNRTI inhibitors.

(ff) A method of inhibiting HIV replication in a subject in need thereof which comprises administering to the subject the pharmaceutical composition of (w) (x) or (y) or the combination of (z) or (aa).

(gg) A method of treating HIV infection and/or reducing the likelihood or severity of symptoms of HIV infection in a subject in need thereof which comprises administering to the subject the pharmaceutical composition of (w) (x) or (y) or the combination of (z) or (aa).

The present invention also includes a compound of the present invention for use (i) in, (ii) as a medicament for, or (iii) in the preparation of a medicament for: (a) medicine; (b) inhibiting HIV replication or (c) treating HIV infection and/or reducing the likelihood or severity of symptoms of HIV infection. In these uses, the compounds of the present invention can optionally be employed in combination with one or more second therapeutic agents selected from HIV antiviral agents, anti-infective agents, and immunomodulators.

Additional embodiments of the invention include the pharmaceutical compositions, combinations and methods set forth in (a)-(gg) above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate.

It is further to be understood that the embodiments of compositions and methods provided as (a) through (gg) above are understood to include all embodiments of the compounds, including such embodiments as result from combinations of embodiments.

Non-limiting examples of the Compounds of Formula (I) include compounds 1-80 as set forth in the Examples below, and pharmaceutically acceptable salts thereof.

### Methods For Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below and generalized in the Schemes below. Alternative synthetic pathways and analogous structures will be apparent to those skilled in the art of organic synthesis.

### General List of Abbreviations

Abbreviations and acronyms employed herein include the following:

| | |
|---|---|
| Ac | Acetyl |
| Aq | Aqueous |
| CAN | Acetonitrile |
| AUC | Area under the curve |
| BAST | Bis(2-methoxyethyl)aminosulfur trifluoride |
| Bu | Butyl |
| Bz | Benzoyl |
| DBDMH | 1,3-Dibromo-5,5-dimethylhydantoin |
| DCM | Dichloromethane |
| DHP | 3,4-dihydro-2H-pyran |
| DIEA, DIPEA or Hünig's base | N,N-diisopropylethylamine |
| DMAP | 4-dimethylaminopyridine |
| DME | dimethyoxyethane |
| DMF | dimethylformamide |
| DMP | Dess-Martin periodinane |
| Dppf | 1,1′-Bis(diphenylphosphino)ferrocene |
| DMSO | dimethyl sulfoxide |
| EDCI | N-ethyl-N′-(3-dimethylaminopropyl)carbodiimide |
| | hydrochloride |
| Et | Ethyl |
| EtOH | Ethanol |
| EtOAc | ethyl acetate |
| G | Grams |
| GI | Gastrointenstinal |
| H | Hour |
| HIV | human immunodeficiency virus |
| HPBCD | hydroxypropyl β-cyclodextrin |
| HPLC | high-performance liquid chromatography |
| mCPBA, CPBA | meta-Chloroperoxybenzoic |
| Hz | Hertz |
| IPA | Isopropanol |
| IV | Intravenous |
| iPr | Isopropyl |
| Ir[dF(CF₃)ppy]₂(dtbpy)PF₆ | [4,4′-Bis(1,1-dimethylethyl)-2,2′-bipyridine-N1,N1]bis[3,5- |
| | difluoro-2-[5-(trifluoromethyl)-2-pyridinyl-N]phenyl- |
| | C]Iridium(III) hexafluorophosphate |
| L | Liter |
| LC | liquid chromatography |
| LC/MS | liquid chromatography mass spectrometry |
| LED | light-emitting diode |
| LiHMDS | lithium bis(trimethylsilyl)amide |
| Me | Methyl |
| MeOH | Methanol |
| Mg | Milligrams |
| MHz | Megahertz |
| Min | Minute |
| μL | Microliters |
| mL | Milliliters |
| Mmol | Millimoles |
| MOM-CI | chloromethyl methyl ether |
| MS | mass spectrometry |
| NBS | N-Bromosuccinimide |
| NHS | normal human serum |
| NIS | N-Iodosuccinimide |
| NMO | 4-methylmorpholine N-oxide |
| NMR | nuclear magnetic resonance spectroscopy |
| PBMC | peripheral blood mononuclear cell |
| Ph | Phenyl |
| P.O. | Oral |
| PTSA | para-toluenesulfonic acid |
| Pr | Propyl |
| Rpm | revolutions per minute |
| RT or rt | room temperature (ambient, about 25° C.) |
| sat or sat'd | Saturated |
| SFC | supercritical fluid chromatography |
| TBAF | Tetra-n-butylammonium fluoride |
| TBDPSC1 | tert-Butyldiphenylchlorosilane |
| tBu | tert-butyl |
| TEA | triethylamine (Et₃N) |
| TEMED | tetramethylethylenediamine |
| TFA | trifluoroacetic acid |
| TFV | Tenofovir |
| TFV-MP | Tenofovir monophosphoate |
| TFV-DP | Tenofovir diphosphate |
| THF | Tetrahydrofuran |
| TMS | Tetramethylsilane |
| UPLC | ultrahigh pressure liquid chromatography |
| UV | Ultraviolet |
| UV/VIS | ultraviolet/visible |
| W | Watt |

### General Procedures

Starting materials and intermediates are purchased or are made using known procedures, or as otherwise illustrated. The general route applied to the synthesis of compounds of Formula I is described in the Schemes that follows. In some cases the order of carrying out the reaction steps in the schemes may be varied to facilitate the reaction or to avoid unwanted reaction products.

Reactions sensitive to moisture or air were performed under nitrogen or argon using anhydrous solvents and reagents. The progress of reactions was determined by either analytical thin layer chromatography (TLC) usually performed with E. Merck pre-coated TLC plates, silica gel 60F-254, layer thickness 0.25 mm or liquid chromatography-mass spectrometry (LC/MS).

Typically the analytical LC-MS system used consisted of a Waters ZQ™ platform with electrospray ionization in positive ion detection mode with an Agilent 1100 series HPLC with autosampler. The column was commonly a Waters Xterra MS C18, 3.0×50 mm, 5 μm or a Waters Acquity UPLC® BEH C18 1.0×50 mm, 1.7 μm. The flow rate was 1 mL/min, and the injection volume was 10 μL. UV detection was in the range 210-400 nm. The mobile phase consisted of solvent A (water plus 0.05% TFA) and solvent B (MeCN plus 0.05% TFA) with a gradient of 100% solvent A for 0.7 min changing to 100% solvent B over 3.75 min, maintained for 1.1 min, then reverting to 100% solvent A over 0.2 min. Alternatively, the column was commonly a Waters Acquity UPLC® BEH C18 1.0×50 mm, 1.7 μm. The flow rate was 0.3 mL/min, and the injection volume was 0.5 μL. UV detection was 215 or 254 nm. Either the mobile phase consisted of solvent A (water plus 0.05% TFA) and solvent B (MeCN plus 0.05% TFA) with a gradient of 90% solvent A changing to 99% solvent B over 1.6 min, maintained for 0.4 min, then reverting to 90% solvent A over 0.1 min or the mobile phase consisted of solvent A (water plus 0.05% TFA) and solvent B (MeCN plus 0.05% TFA) with a gradient of 97% solvent A changing to 4% then 50% solvent B over 0.5 min and 0.9 min, 50%-99% solvent B over 0.2 min, maintained for 0.4 min, then reverting to 90% solvent A over 0.1 min.

Preparative HPLC purifications were usually performed using either a mass spectrometry directed system or anon-mass guided system. Usually they were performed on a Waters Chromatography Workstation configured with LC-MS System consisting of: Waters ZQ™ single quad MS system with Electrospray Ionization, Waters 2525 Gradient Pump, Waters 2767 Injecto/Collector, Waters 996 PDA Detector, the MS Conditions of: 150-750 amu, Positive Electrospray, Collection Triggered by MS, and a Waters SUNFIRE® C-18 5 micron, 30 mm (id)×100 mm column. The mobile phases consisted of mixtures of acetonitrile (10-100%) in water containing 0.1% TFA. Flow rates were maintained at 50 mL/min, the injection volume was 1800 μL, and the UV detection range was 210-400 nm. An alternate preparative HPLC system used was a Gilson Workstation consisting of. Gilson GX-281 Injector/Collector, Gilson UV/VIS-155 Detector, Gilson 322, 333, and 334 Pumps, and a Phenomenex Gemini-NX C-18 5 micron, 50 mm (id)×250 mm column, a Waters XBridge™ C-18 5 micron OBD™, 30 mm (id)×250 mm column, or a Waters SUNFIRE™ C-18 OBD™ 10 micron, 30 mm (id)×150 mm column. The mobile phases consisted of mixtures of acetonitrile (0-90%) in water containing 0.1% or 0.05% TFA. Flow rates were maintained at 50 mL/min for the Waters Xbridge™ column, 90 mL/min for the Phenomenex Gemini column, and 30 mL/min for the Waters SUNFIRE™ column. The injection volume ranged from 1000-8000 μL, and the UV detection range was 210-400 nm. Mobile phase gradients were optimized for the individual compounds. Reactions performed using microwave irradiation were normally carried out using an Emrys Optimizer manufactured by Personal Chemistry, or an Initiator manufactured by Biotage. Reactions performed using photon irradiation were normally carried out using either a second generation Merck photoreactor or a Kessil 34 W blue LED lamp. Concentration of solutions was carried out on a rotary evaporator under reduced pressure. Flash chromatography was usually performed using either a Biotage® Flash Chromatography apparatus (Dyax Corp.), an ISCO CombiFlash® Rf apparatus, or an ISCO CombiFlash® Companion XL on silica gel (32-63 microns, 60 Å pore size) in pre-packed cartridges of the size noted. ¹H NMR spectra were acquired at 500 MHz spectrometers in CDCl₃ solutions unless otherwise noted. Chemical shifts were reported in parts per million (ppm). Tetramethylsilane (TMS) was used as internal reference in CD₃Cl solutions, and residual CH₃OH peak or TMS was used as internal reference in CD₃OD solutions. Coupling constants (J) were reported in hertz (Hz). Chiral analytical chromatography was most commonly performed on one of CHIRALPAK® AS, CHIRALPAK® AD, CHIRALCEL® OD, CHIRALCEL® IA, or CHIRALCEL® OJ columns (250×4.6 mm) (Daicel Chemical Industries, Ltd.) with noted percentage of ethanol in hexane (% EtOH/Hex), isopropanol in heptane (% IPA/Hep), ethanol in carbon dioxide (% EtOH/CO₂), or isopropanol in carbon dioxide (% IPA/CO₂) as isocratic solvent systems. Chiral preparative chromatography was conducted on one of CHIRALPAK AS, of CHIRALPAK AD, CHIRALCEL® OD, CHIRALCEL®IA, CHIRALCEL® OJ columns (20×250 mm) (Daicel Chemical Industries, Ltd.) with desired isocratic solvent systems identified on chiral analytical chromatography or by supercritical fluid (SFC) conditions.

Several methods for preparing the compounds of this invention are also described in the Examples. Starting materials and intermediates were purchased commercially from common catalog sources or were made using known procedures, or as otherwise illustrated.

### Example 1

### Preparation of Intermediate Compound Int-1

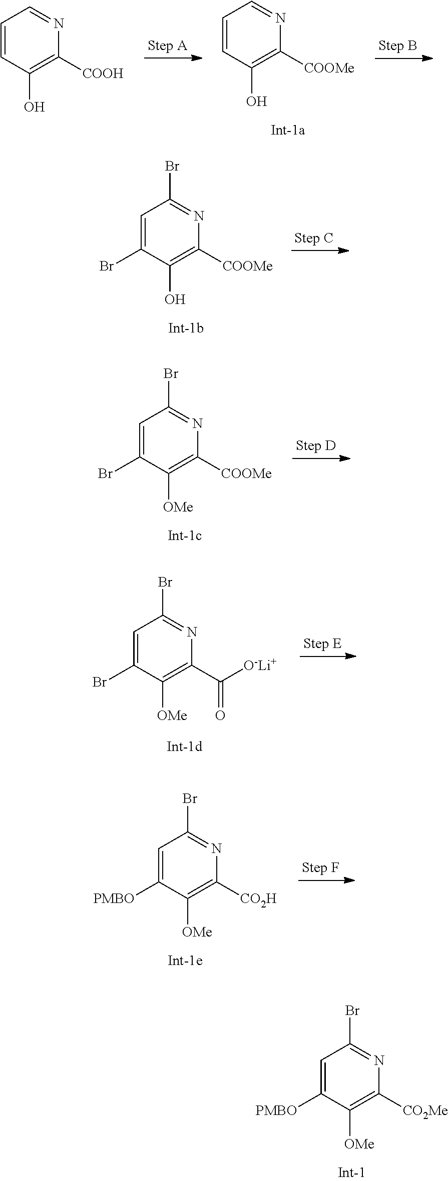

### Step A—Synthesis of Compound Int-1a

To a solution of 3-hydroxypicolinic acid (340 g, 2.44 mol) in 2.8 L of MeOH stirred at 15° C., was added H₂SO₄ (720 g, 7.33 mol). The reaction was heated to 65° C. by an oil bath and stirred for 2 hours. After it was cooled to room temperature, the reaction content was neutrolized to pH=7 by slow addition of saturated Na₂CO₃ aqueous solution. The resulting mixture was extracted with EtOAc. The combined organic layers were washed with brine, dried over anhydrous Na₂SO₄. After filtration, the filtrate was concentrated under vacuum to give compound Int-1a. The crude material was used in the next reaction without further purification. ¹HNMR (400 MHz, CDCl₃) δ 10.62 (s, 1H), 6.28 (d, J=4.4 Hz, 2H), 4.05 (s, 3H).

### Step B—Synthesis of Compound Int-1b

To a mixture of compound Int-1a (50 g, 327 mmol) in water (5.0 L) stirred at 15° C., was added bromine (157 g, 979 mmol). The mixture was stirred at 15° C. for 5 hours. The resulting mixture was filtered, and the filter cake was washed with water and dried under vacuum to give compound Int-1b. The crude material was used in the next reaction without further purification. ¹H NMR (400 MHz, CDCl₃) δ 11.37 (s, 1H), 7.87 (s, 1H), 4.07 (s, 3H).

### Step C—Synthesis of Compound Int-1c

To a solution of compound Int-1b (200 g, 643 mmol) in acetone (4.0 L) stirred at 15° C., was added Cs₂CO₃ (377 g, 1.160 mol) followed by dropwise addition of iodomethane (274 g, 1930 mmol). The reaction was heated at 60° C. for 2 hours. After it was cooled to room temperature, the reaction mixture was filtered. The filter cake was washed with acetone, and purified by silica gel chromatography eluting with petroleum ether: EtOAc=25:110:1 to give compound Int-1c. ¹H NMR (400 MHz, CDCl₃) δ 7.85 (s, 1H), 3.99 (s, 3H), 3.98 (s, 3H).

### Step D—Synthesis of Compound Int-1d

To a solution of compound Int-1c (350 g, 1080 mmol) in THF (1.8 L) stirred at 15° C., was added water (350 mL) followed by lithium hydroxide monohydrate (54 g, 1300 mmol). The reaction mixture was stirred at 25° C. for 2 hours. The solvent was removed under vacuum to give compound Int-1d. The crude material was used in the next reaction without further purification. ¹H NMR (400 MHz, DMSO-d₆) δ 7.73 (s, 1H), 3.83 (s, 3H).

### Step E—Synthesis of Compound Int-1e

To a solution of compound Int-1d (240 g, 757 mmol) and DMF (1.50 L) stirred at 0˜5° C., was slowly added NaH (115 g, 2.88 mol, 60% wt.). It was stirred at 0˜5° C. for 30 min, and then a solution of (4-methoxyphenyl)methanol (157 g, 1.14 mol) in DMF (1.50 L) was added. The reaction was stirred at 0˜5° C. for 30 min, then warmed to 15° C. and stirred for 2 hours. The reaction was quenched by adding 1 L of saturated NH₄Cl aqueous solution, and acidified with 4 N HCl aqueous solution until pH=4˜5. The resulting mixture was extracted with EtOAc. The organic layer was washed with brine, dried over anhydrous Na₂SO₄, and then concentrated under vacuum to give compound Int-1e. Mass Calc'd for C₁₅H₁₄NBrO₅: 367.0, found 389.8 (M+Na)⁺.

### Step F—Synthesis of Compound Int-1f

To a mixture of compound Int-1e (290 g, 788 mmol) and K₂CO₃ (272 g, 1970 mmol) in DMF (2.5 L) stirred at 15° C., was slowly added iodomethane (355 g, 2360 mmol). The reaction was stirred at 15° C. for 12 h. The reaction mixture was diluted with 1.5 L of water and extracted with EtOAc. The organic layer was washed with brine, dried over anhydrous Na₂SO₄, then concentrated under vacuum. The residue was purified by silica gel chromatography eluting with petroleum ether:EtOAc:dichloromethane=10:1˜2:1. The product containing fractions were combined and concentrated under vacuum. The residue was recrystallized from EtOAc/petroleum ether. The solid was collected by filtration, washed with petroleum ether, and dried under vacuum to give compound Int-1. ¹H NMR (400 MHz, CDCl₃): δ 7.35 (d, J=8.8 Hz, 2H), 7.16 (s, 1H), 6.95 (d, J=8.8 Hz, 2H), 5.10 (s, 2H), 3.95 (s, 3H), 3.91 (s, 3H), 3.84 (s, 3H).

### Example 2

### Preparation of Compound Int-2e

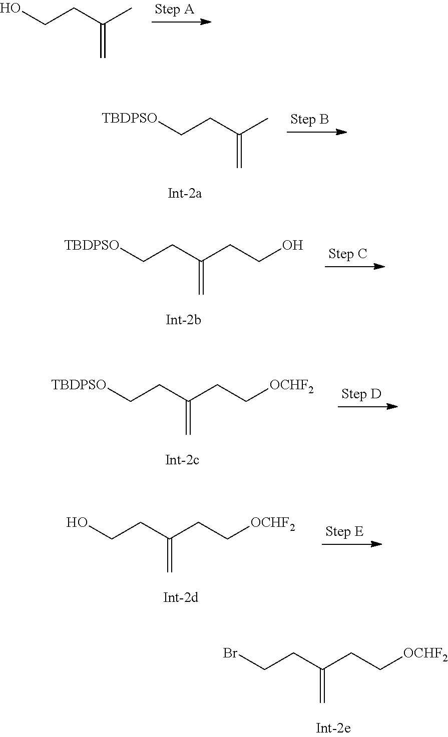

### Step A Synthesis of Compound Int-2a

To a solution of 3-methylbut-3-en-1-ol (20 g, 232 mmol) in DCM (300 mL) was added imidazole (31.6 g, 464 mmol) and TBDPSCl (89 mL, 348 mmol) in portions at 0° C. The solution was stirred at 25° C. for 5 hours before being quenched with water (80 mL) and separated. The aqueous layer was extracted with EtOAc (3×60 mL). The combined organic phase was dried over anhydrous Na₂SO₄, filtered, and the filtrate was concentrated in vacuo. The residue was purified by column chromatography on silica gel (200 g) eluting with 100% petroleum ether to afford compound Int-2a. ¹H NMR (400 MHz, CDCl₃) δ: 7.67 (dd, J=7.8, 1.7 Hz, 4H); 7.45-7.35 (m, 6H); 4.78-4.64 (m, 2H); 3.76 (t, J=6.9 Hz, 2H); 2.28 (t, J=6.8 Hz, 2H); 1.68 (s, 3H); 1.04 (s, 9H).

### Step B—Synthesis of Compound Int-2b

To a mixture of compound Int-2a (10 g, 30.8 mmol) and paraformaldehyde (1.018 g, 33.9 mmol) in DCM (150 mL) was added dropwise a solution of 1 M dimethylaluminum chloride in heptane (40.1 mL, 40.1 mmol) at 0° C. The mixture was stirred at 0° C. for 2 hours before being quenched with water (40 mL). 1 N aqueous HCl was added dropwise to dissolve the precipitate. The mixture was filtered and the filtrate was separated. The aqueous layer was extracted with EtOAc (3×40 mL). The combined organic layers were dried over sodium sulfate, filtered, and concentrated in vacuo. The residue was purified by silica gel chromatography eluting with 10% EtOAc/petroleum ether to afford compound Int-2b. ¹H NMR (400 MHz, CDCl₃) δ: 7.70-7.64 (m, 4H); 7.44-7.35 (m, 6H); 4.88 (s, 2H); 3.77 (t, J=6.7 Hz, 2H); 3.66 (t, J=6.2 Hz, 2H); 2.31-2.23 (m, 4H); 1.04 (s, 9H).

### Step C—Synthesis of Compound Int-2c

To a mixture of compound Int-2b (3 g, 8.46 mmol) and potassium acetate (3.32 g, 33.8 mmol) in DCM (4 mL) and water (4 mL) was added (bromodifluoromethyl)trimethylsilane (3.44 g, 16.92 mmol) at 25° C. under a nitrogen balloon. The mixture was stirred at 25° C. for 15 hours before being diluted with water (5 mL) and extracted with EtOAc (3×15 mL). The combined organic layers were dried over anhydrous sodium sulfate, filtered, and concentrated in vacuo. The residue was purified by flash silica gel chromatography (40 g column) eluting with 0-5% EtOAc/petroleum ether to afford compound Int-2c. ¹H NMR (400 MHz, CDCl₃) δ: 7.66 (dd, J 7.7, 1.5 Hz, 4H); 7.43-7.36 (m, 6H); 6.39-5.94 (m, 1H); 4.83 (br s, 2H); 3.89 (t, J=7.0 Hz, 2H); 3.75 (t, J=6.8 Hz, 2H); 2.30 (dt, J=12.5, 6.5 Hz, 4H); 1.04 (s, 9H).

### Step D—Synthesis of Compound Int-2d

To a mixture of compound Int-2c (8 g, 19.77 mmol) in THF (80 mL) was added a solution of 1 M TBAF in THF (23.73 mL, 23.73 mmol). The mixture was stirred at 15° C. for 2 hours before being concentrated in vacuo. The residue was purified by flash silica gel chromatography (80 g column) eluting with 0-15% EtOAc/petroleum ether to afford compound Int-2d. ¹H NMR (400 MHz, CDCl₃) δ: 6.20 (t, J=75.0 Hz, 1H); 4.95 (d, J=6.8 Hz, 2H); 3.96 (t, J=13.0 Hz, 2H); 3.48 (t, J=7.2 Hz, 2H); 2.61 (t, J=7.6 Hz, 2H); 2.38 (t, J=6.4 Hz, 2H).

### Step E—Synthesis of Compound Int-2e

To a stirred solution of compound Int-2d (4.2 g, 25.3 mmol) in DCM (40 mL) was added triphenylphosphine (7.96 g, 30.3 mmol) and carbon tetrabromide (10.90 g, 32.9 mmol). The mixture was stirred at 20° C. for 1 hour before being concentrated in vacuo. The residue was purified by flash silica gel chromatography (40 g column) eluting with 0-5% EtOAc/petroleum ether to afford compound Int-2e. ¹H NMR (400 MHz, CDCl₃) δ: 6.21 (t, J=74.4 Hz, 1H); 4.96 (d, J=7.2 Hz, 2H); 3.97 (t, J=6.8 Hz, 2H); 3.48 (t, J=6.8 Hz, 2H); 2.63 (t, J=7.2 Hz, 2H); 2.40 (t, J=6.8 Hz, 2H).

### Example 3

### Preparation of Compounds 1-4

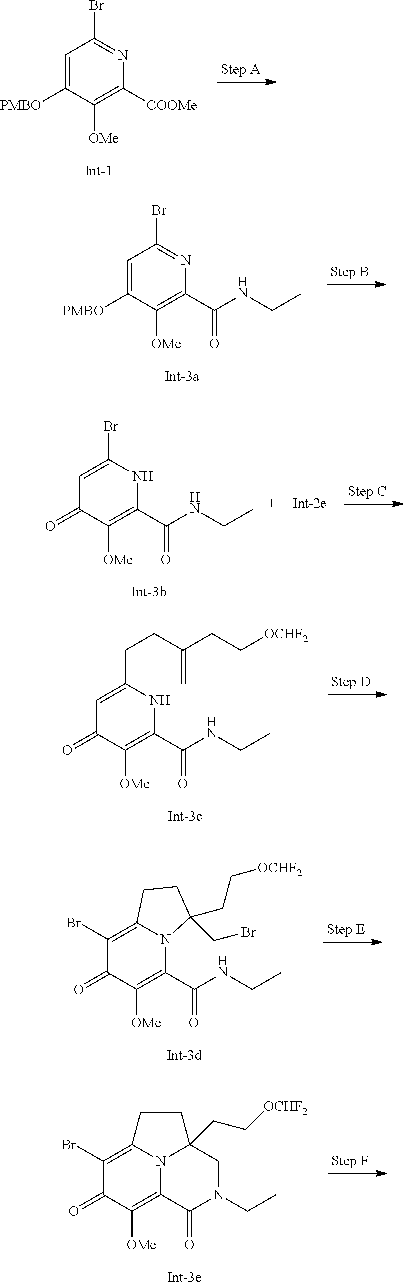

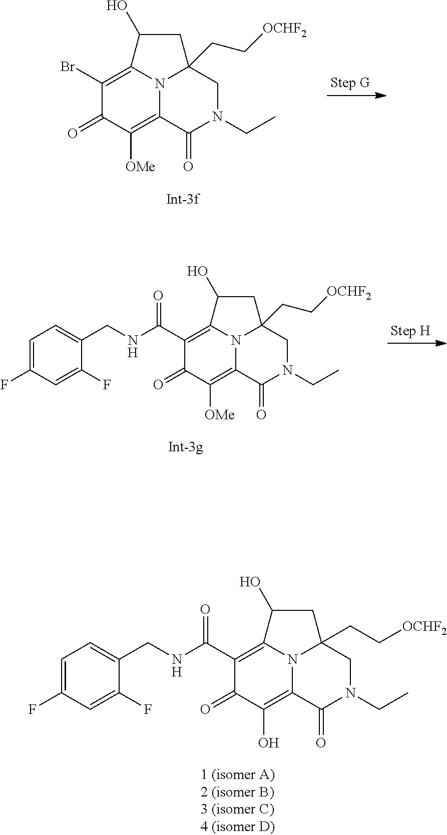

### Step A—Synthesis of Compound Int-3a

To a stirred solution of compound Int-1 (10 g, 26.2 mmol) in THF (3 mL) was added ethanamine (30 mL, 26.2 mmol, THF solvent). The mixture was stirred at 20° C. for 5 hours before being concentrated under reduced pressure to give compound Int-3a. LCMS anal. calcd. for C₁₇H₁₉BrN₂O₄: 394.1, 396.1; Found: 395.0, 397.0 (M+1)⁺.

### Step B—Synthesis of Compound Int-3b

To a stirred solution of compound Int-3a (10 g, 25.3 mmol) in DCM (50 mL) was added TFA (10 mL). The mixture was stirred at 20° C. for 2 hours before being concentrated under reduced pressure. The residue was purified by silica gel chromatography eluting with 10% MeOH/DCM to give compound Int-3b. LCMS anal. calcd. for C₉H₁₁BrN₂O₃: 274.0, 276.0; Found: 275.0, 277.0 (M+1)⁺.

### Step C—Synthesis of Compound Int-3c

A vial equipped with a magnetic stirring bar (vial A) was charged with Ir[dF(CF₃)ppy]₂(dtbpy)PF₆ (8.16 mg, 7.27 μmol), compound Int-3b (200 mg, 0.727 mmol), sodium carbonate (154 mg, 1.454 mmol), and tris(trimethylsilyl)silane (542 mg, 2.181 mmol). Meanwhile, a separate vial (vial B) was sequentially charged with nickel(II) chloride ethylene glycol dimethyl ether complex (37 mg, 0.168 mmol), 4,4′-di-tert-butyl-2,2′-bipyridine (45 mg, 0.168 mmol), and 16 mL of MeCN, and the mixture was sonicated until homogeneous (˜15 minutes). 7.3 mL of this stock solution in vial B was added to vial A containing the other reaction components. The reaction mixture was degassed via sparging with nitrogen for 10 minutes. Compound Int-2e (500 mg, 2.181 mmol) was added before the vial was sealed with parafilm. The vial was then placed in front of a Kessil 34 W blue LED lamp. The reaction was allowed to stir with irradiation for 4 hours before being filtered. The filtrate was concentrated in vacuo and the residue was purified by flash silica gel chromatography (20 g column) eluting with 0-10% MeOH/DCM to afford compound Int-3c. LCMS anal. calcd. for C₁₆H₂₂F₂N₂O₄: 344.2; Found: 345.2 (M+1)⁺.

### Step D—Synthesis of Compound Int-3d

To a stirred solution of compound Int-3c (380 mg, 1.104 mmol) in THF (5 mL) was added NBS (393 mg, 2.207 mmol). The mixture was stirred at 15° C. for 0.5 hours before being concentrated in vacuo. The residue was purified by flash silica gel chromatography (12 g column) eluting with 0-10% MeOH/DCM to afford compound Int-3d. LCMS anal. calcd. for C₁₆H₂Br₂F₂N₂O₄: 502.0; Found: 503.0 (M+1)⁺.

### Step E—Synthesis of Compound Int-3e

A mixture of compound Int-3d (570 mg, 1.135 mmol) and Cs₂CO₃ (1110 mg, 3.41 mmol) in DMF (10 mL) was stirred at 20° C. for 9 hours before being filtered. The filtrate was concentrated in vacuo and the residue was purified by flash silica gel chromatography (20 g column) eluting with 0-10% MeOH/DCM to afford compound Int-3e. LCMS anal. calcd. for C₁₆H₉BrF₂N₂O₄: 420.1, 422.1; Found: 420.9, 422.9 (M+1)⁺.

### Step F—Synthesis of Compound Int-3f

To a solution of compound Int-3e (160 mg, 0.380 mmol) in THF (16 mL) was added LiHMDS 1 M in THF (1.140 mL, 1.140 mmol) at −78° C. After 20 minutes, to the mixture was added a solution of 3-phenyl-2-(phenylsulfonyl)-1,2-oxaziridine (198 mg, 0.760 mmol) in THF (0.5 mL) at −78° C. The mixture was stirred at 16° C. for 20 minutes before being quenched with MeOH (2 mL) and concentrated in vacuo. The residue was purified by flash silica gel chromatography (12 g column) eluting with 0-10% MeOH/DCM to afford compound Int-3f. LCMS anal. calcd. for C₁₆H₁₉BrF₂N₂O₅: 436.0, 438.0; Found: 437.1, 439.1 (M+1)⁺.

### Step H—Synthesis of Compound Int-3g

To a solution of compound Int-3f (40 mg, 0.091 mmol) in DMSO (1.5 mL) and MeOH (0.5 mL) was added (2,4-difluorophenyl)methanamine (39.3 mg, 0.274 mmol), N-ethyl-N-isopropylpropan-2-amine (59.1 mg, 0.457 mmol), and Pd(Ph₃P)₄ (52.9 mg, 0.046 mmol). The mixture was degassed and purged with CO three times. The resulting mixture was stirred at 120° C. under CO (15 psi). After 2 hours, the mixture was diluted with EtOAc (20 mL) and washed with water (5 mL) and brine (5 mL). The organic layer was dried over sodium sulfate, filtered, and concentrated in vacuo. The residue was purified by preparative TLC plate eluting with 100% EtOAc to afford the product, which was further purified by preparative SFC (DAICEL CHIRALPAK AD-H, 5 μm, 30×250 mm column, 60 mL/min, 40% (EtOH+0.1% NH₃H₂O)/CO₂) to afford Isomer A of compound Int-3g (1^{st} eluting component), Isomer B of compound Int-3g (2^{nd} eluting component), Isomer C of compound Int-3g (3^{rd} eluting component), and Isomer D of compound Int-3g (4^{th} eluting component). Isomer C of compound Int-3g was further purified by preparative SFC (DAICEL CHIRALCEL OJ-H, 5 μm, 30×250 mm column, 60 mL/min, 30% (EtOH+0.1% NH₃H₂O)/CO₂) to afford Isomer C of compound Int-3g. Isomer D of compound Int-3g was further purified by preparative SFC (DAICEL CHIRALCEL OJ-H, 5 μm, 30×250 mm column, 60 mL/min, 30% (EtOH+0.1% NH₃H₂O)/CO₂) to afford Isomer D of compound Int-3g. LCMS anal. calcd. for C₂₄H₂₅F₄N₃O₆: 527.2; Found: 528.1 (M+1)⁺.

### Step H—Synthesis of Compound 1, Compound 2, Compound 3, and Compound 4

To a stirred solution of Isomer A of compound Int-3g (7 mg, 0.013 mmol) in acetonitrile (1 mL) was added magnesium bromide (12.22 mg, 0.066 mmol). The mixture was stirred at 30° C. for 2 hours before being purified by preparative reverse phase HPLC (Boston Green ODS, 5 μm, 30×150 mm column) eluting with 30-60% ACN/(water+0.1% TFA). After lyophilization, the product was co-evaporated with toluene (2×10 mL) to afford compound 1. ¹H NMR (400 MHz, CD₃OD) δ: 7.48-7.37 (m, 1H); 7.00-6.86 (m, 2H); 6.49-6.06 (m, 1H); 5.69 (t, J=7.9 Hz, 1H); 4.63 (br s, 2H); 3.99-3.83 (m, 4H); 3.73 (br dd, J=13.4, 6.8 Hz, 1H); 3.50-3.61 (m, 1H); 3.02 (dd, J=13.1, 7.7 Hz, 1H); 2.23-1.97 (m, 3H); 1.24 (t, J=7.2 Hz, 3H). LCMS anal. calcd. for C₂₃H₂₃F₄N₃O₆: 513.2; Found: 514.0 (M+1)⁺.

Following essentially the method employed to produce compound 1 in step H of example 3, compound 2 was prepared from Isomer B of compound Int-3g. ¹H NMR (400 MHz, CD₃OD) δ: 7.48-7.39 (m, 1H); 6.99-6.89 (m, 2H); 6.55-6.12 (m, 1H); 5.73 (d, J=7.6 Hz, 1H); 4.62 (s, 2H); 4.02-4.15 (m, 2H); 3.94-3.87 (m, 1H); 3.84-3.72 (m, 2H); 3.50 (dq, J=13.8, 7.1 Hz, 1H); 2.55-2.48 (m, 1H); 2.43-2.28 (m, 3H); 1.24 (t, J=7.2 Hz, 3H). LCMS anal. calcd. for C₂₃H₂₃F₄N₃O₆: 513.2; Found: 514.0 (M+1)⁺.

Following essentially the method employed to produce compound 1 in step H of example 3, compound 3 was prepared from Isomer C of compound Int-3g. ¹H NMR (400 MHz, CD₃OD) δ: 7.43 (br d, J=6.8 Hz, 1H); 7.00-6.87 (m, 2H); 6.57-6.12 (m, 1H); 5.73 (br d, J=7.3 Hz, 1H); 4.62 (br s, 2H); 4.09 (br d, J=5.1 Hz, 2H); 3.94-3.87 (m, 1H); 3.86-3.75 (m, 2H); 3.53-3.46 (m, 1H); 2.56-2.47 (m, 1H); 2.44-2.30 (m, 3H); 1.24 (br t, J=7.2 Hz, 3H). LCMS anal. calcd. for C₂₃H₂₃F₄N₃O₆: 513.2; Found: 514.0 (M+1)⁺.

Following essentially the method employed to produce compound 1 in step H of example 3, compound 4 was prepared from Isomer D of compound Int-3g. ¹H NMR (400 MHz, CD₃OD) δ: 7.42 (br d, J=8.3 Hz, 1H); 6.94 (br d, J=11.2 Hz, 2H); 6.49-6.06 (m, 1H); 5.75-5.62 (m, 1H); 4.63 (br s, 2H); 3.91 (br d, J=12.5 Hz, 4H); 3.72 (br s, 1H); 3.61-3.52 (m, 1H); 3.09-2.97 (m, 1H); 2.24-2.04 (m, 3H); 1.24 (br t, J=7.3 Hz, 3H). LCMS anal. calcd. for C₂₃H₂₃F₄N₃O₆: 513.2; Found: 514.0 (M+1)⁺.

### Example 4

### Preparation of Compounds 5-8

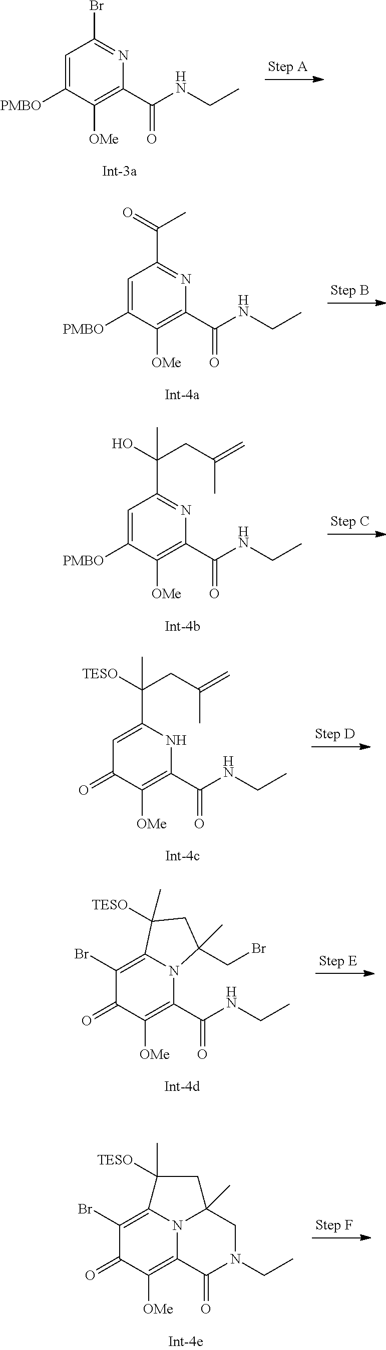

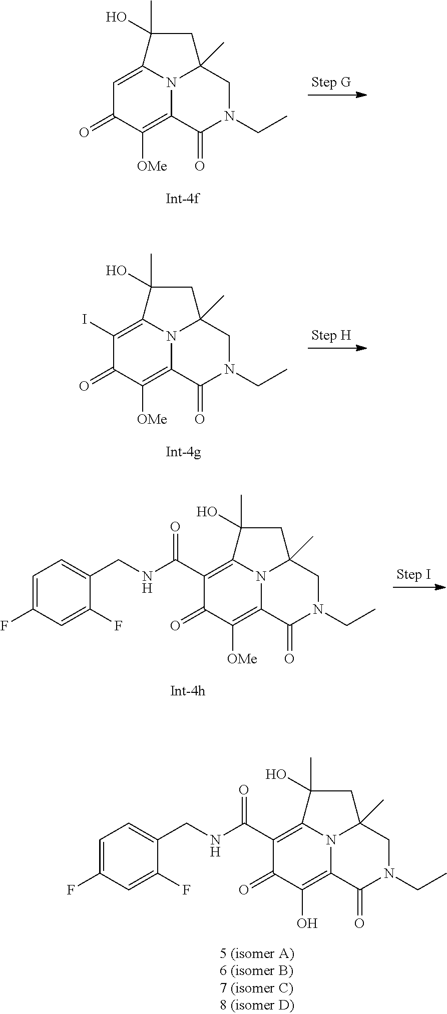

### Step A—Synthesis of Compound Int-4a

To a solution of compound Int-3a (5 g, 12.65 mmol) in acetonitrile (100 mL) was added tributyl(1-ethoxyvinyl)stannane (5.13 mL, 15.18 mmol) and bis(triphenylphosphine)palladium(II) dichloride (0.888 g, 1.265 mmol). The mixture was sparged with nitrogen for 5 minutes before being heated at 75° C. overnight. The reaction was cooled to room temperature prior to the addition of phosphoric acid (12.65 mL, 12.65 mmol). The reaction was stirred for 1 hour before saturated aqueous sodium bicarbonate (150 mL) was added. The mixture was extracted with EtOAc (3×100 mL). The combined organic layers were washed with brine, dried over magnesium sulfate, filtered, and concentrated. The residue was purified by flash silica gel chromatography (220 g column) eluting with 0-100% EtOAc/hexanes to afford compound Int-4a. LCMS anal. calcd. for C₁₉H₂₂N₂O₅: 358.15; Found: 359.12 (M+1)⁺.

### Step B—Synthesis of Compound Int-4b

To a solution of compound Int-4a (2.0 g, 5.58 mmol) in tetrahydrofuran (50 mL) was added 3-bromo-2-methylprop-1-ene (1.507 g, 11.16 mmol), sodium iodide (1.673 g, 11.16 mmol), and indium (1.281 g, 11.16 mmol) under a N₂ atmosphere. The mixture was stirred at room temperature for 30 minutes before being heated to 70° C. for 1 hour. The reaction was cooled to room temperature and filtered. The filtrate was concentrated and the residue was purified by flash silica gel chromatography (80 g column) eluting with 10% MeOH/DCM to afford compound Int-4b. LCMS anal. calcd. for C₂₃H₃₀N₂O₅: 414.22; Found: 415.30 (M+1)⁺.

### Step C—Synthesis of Compound Int-4c

To a solution of compound Int-4b (2.0 g, 4.83 mmol) in dichloromethane (20 mL) was added trifluoacetic acid (2 mL, 26.1 mmol). The mixture was stirred at room temperature for 1 hour before being concentrated. To a solution of the resulting residue in N,N-dimethylformamide (10 mL) was added imidazole (0.657 g, 9.65 mmol), followed by chlorotriethylsilane (1.09 g, 7.24 mmol). The resulting mixture was stirred at 50° C. for 2 hours before being concentrated. The resulting residue was purified by silica gel column chromatography (80 g column) eluting with eluting with 0-10% MeOH/DCM to afford compound Int-4c. LCMS anal. calcd. for C₂₁H₃₆N₂O₄Si: 408.24; Found: 409.34 (M+1)⁺.

### Step D—Synthesis of Compound Int-4d

To a stirred solution of compound Int-4c (1.0 g, 2.447 mmol) in acetonitrile (25 mL) was added 1-bromopyrrolidine-2,5-dione (1.089 g, 6.12 mmol). The mixture was stirred at room temperature for 1.5 hours before being concentrated. The residue was taken up in 50% EtOAc/hexanes (3 mL) and filtered. The filtrate was concentrated and the resulting residue was purified by C18 reverse phase chromatography (80 g column) eluting with 10-100% (ACN/water)+0.05% TFA to afford compound Int-4d. LCMS anal. calcd. for C₂₁H₃₄Br₂N₂O₄Si: 566.06; Found: 567.06 (M+1)⁺.

### Step E—Synthesis of Compound Int-4e

To a stirred solution of compound Int-4d (720 mg, 1.271 mmol) in dimethyl sulfoxide (12 mL) was added cesium carbonate (621 mg, 1.907 mmol). The mixture was stirred at room temperature for 1.5 hours before being directly purified on a C18 reverse phase column eluting with 0-100% (ACN/water)+0.05% TFA to afford compound Int-4e. LCMS anal. calcd. for C₂₁H₃₃BrN₂O₄Si: 484.14; Found: 485.13 (M+1)⁺.

### Step F—Synthesis of Compound Int-4f

Hydrochloric acid 4 M in dioxane (0.520 mL, 2.080 mmol) was added to a stirred solution of compound Int-4e (0.5 g, 1.04 mmol) in methanol (15 mL). The reaction mixture was stirred at room temperature for 2 hours before being concentrated under reduced pressure. The resulting residue was re-dissolved in methanol (15 mL) and palladium on carbon (10% wt.) (0.111 g, 0.104 mmol) was added prior to placing the mixture under an H₂ balloon. After 2 hours, the reaction was filtered and concentrated under reduced pressure. The residue was purified by flash silica gel chromatography (40 g column) eluting with 0-30% MeOH/DCM to afford compound Int-4f. LCMS anal. calcd. for C₁₅H₂₀N₂O₄: 292.14; Found: 293.12 (M+1)⁺.

### Step G—Synthesis of Compound Int-4g

N-iodosuccinimide (142 mg, 0.631 mmol) and 3-choloroperbenzoic acid (136 mg, 0.631 mmol) were added to a stirred solution of compound Int-4f (123 mg, 0.421 mmol) in methanol (5 mL). The reaction mixture was heated at 70° C. for 2 hours before being cooled to room temperature and concentrated under reduced pressure. The resulting residue was purified by preparative TLC plate eluting with 10% MeOH/DCM to afford compound Int-4g. LCMS anal. calcd. for C₁₅H₁₉₁N₂O₄: 418.04; Found: 419.00 (M+1)⁺.

### Step H—Synthesis of Compound Int-4h

Tetrakis(triphenylphosphine)palladium(0) (61 mg, 0.053 mmol), N,N-diisopropylethylamine (184 μl, 1.052 mmol), and 2,4-difluorobenzylamine (75 mg, 0.526 mmol) were added to a stirred solution of compound Int-4g (110 mg, 0.263 mmol) in dimethyl sulfoxide (2 mL). The reaction mixture was degassed and placed under a carbon monoxide atmosphere. The resulting reaction mixture was stirred at 90° C. for 1 hour before being cooled to room temperature, filtered through a 0.45 μm syringe filter, diluted with methanol, and purified by reverse phase HPLC (RediSep Rf C18, 100 g column) eluting with 10-100% (ACN/water)+0.05% TFA to afford the product, which was further purified by chiral preparative SFC (ChiralPak AD-H, 21×250 mm column, 70 g/min, 120 bar, 25% EtOH/CO₂, 40° C.) to afford Isomer A of compound Int-4h (1^{st} eluting component), Isomer B of compound Int-4h (2^{nd} eluting component), Isomer C of compound Int-4h (3^{rd} eluting component), and Isomer D of compound Int-4h (4^{th} eluting component). LCMS anal. calcd. for C₂₃H₂₅F₂N₃O₅: 461.18; Found: 462.41 (M+1)⁺.

### Step I—Synthesis of Compound 5, Compound 6, Compound 7, and Compound 8

Isomer A of compound Int-4h (19.0 mg, 0.041 mmol), magnesium bromide (114 mg, 0.618 mmol) and acetonitrile (1.0 mL) were combined and stirred at room temperature. After 30 minutes, the reaction mixture was diluted with MeOH and filtered through a 0.45 μm syringe filter before being purified by reverse phase HPLC (Waters Sunfire C18 OBD, 10 μm, 30×150 mm column). Product fractions were combined, frozen and lyophilized to afford compound 5. ¹H NMR (500 MHz, CDCl₃) δ 11.48 (s, 1H); 8.20 (br s, 1H); 7.35-7.25 (m, 1H); 6.82-6.78 (m, 2H); 4.66-4.65 (m, 2H); 3.83-3.79 (d, J=20 Hz, 1H); 3.74 (m, 1H); 3.69 (m, 1H); 3.50-3.47 (m, 1H); 2.46 (m, 2H); 1.86 (s, 3H); 1.54 (s, 3H); 1.26 (t, J=5.0 Hz, 3H). LCMS anal. calcd. for C₂₂H₂₃F₂N₃O₅: 447.16; Found: 448.13 (M+1)⁺.

Following essentially the method employed to produce compound 5 in step I of example 4, compound 6 was prepared from Isomer B of compound Int-4h. ¹H NMR (500 MHz, CDCl₃) δ 10.75 (s, 1H); 7.40-7.26 (m, 1H); 6.84-6.81 (m, 2H); 4.72-4.68 (m, 2H); 4.60-4.56 (m, 2H); 3.76-3.71 (m, 2H); 3.58-3.50 (m, 2H); 2.59-2.56 (d, J=15 Hz, 1H); 2.13-2.10 (s, J=15 Hz, 1H); 1.73 (s, 3H); 1.65 (s, 3H); 1.27 (t, J=10 Hz, 3H). LCMS anal. calcd. for C₂₂H₂₃F₂N₃O₅: 447.16; Found: 448.40 (M+1)⁺.

Following essentially the method employed to produce compound 5 in step I of example 4, compound 7 was prepared from Isomer C of compound Int-4h. ¹H NMR (500 MHz, CDCl₃) δ 10.75 (s, 1H); 7.41-7.35 (m, 1H); 6.86-6.78 (m, 2H); 4.73-4.68 (m, 2H); 4.60-4.56 (m, 2H); 3.77-3.70 (m, 2H); 3.57-3.50 (m, 2H); 2.59-2.56 (d, J=15 Hz, 1H); 2.13-2.10 (s, J=15 Hz, 1H); 1.72 (s, 3H); 1.65 (s, 3H); 1.27 (t, J=10 Hz, 3H). LCMS anal. calcd. for C₂₂H₂₃F₂N₃O₅: 447.16; Found: 448.42 (M+1)⁺.

Following essentially the method employed to produce compound 5 in step I of example 4, compound 8 was prepared from Isomer D of compound Int-4h. ¹H NMR (500 MHz, CDCl₃) δ 11.48 (s, 1H); 8.22 (broad, 1H); 7.36-7.33 (m, 1H); 6.82-6.80 (m, 2H); 4.65 (m, 2H); 3.83-3.79 (d, J=20 Hz, 1H); 3.74 (m, 1H); 3.71 (m, 1H); 3.50-3.47 (m, 1H); 2.46 (m, 2H); 1.86 (s, 3H); 1.54 (s, 3H); 1.26 (t, J=5.0 Hz, 3H). LCMS anal. calcd. for C₂₂H₂₃F₂N₃O₅: 447.16; Found: 448.13 (M+1)⁺.

### Example 5

### Preparation of Compounds 9-12

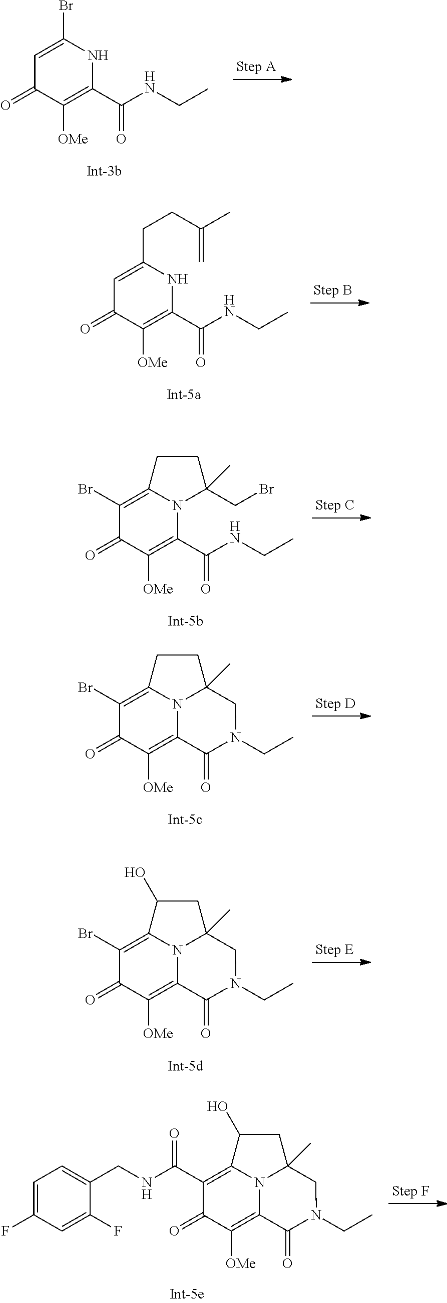

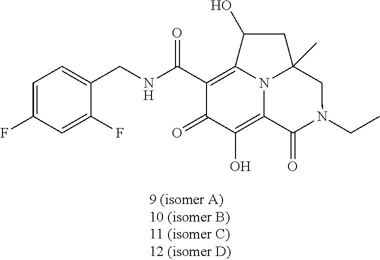

### Step A Synthesis of Compound Int-5a

A 40 mL vial equipped with a magnetic stirring bar was charged with Ir[dF(CF₃)ppy]2(dtbpy)PF₆ (12.23 mg, 10.91 μmol), compound Int-3b (300 mg, 1.091 mmol), sodium carbonate (231 mg, 2.181 mmol), and 1,1,1,3,3,3-hexamethyl-2-(trimethylsilyl)trisilane (1.009 mL, 3.27 mmol). Meanwhile, a separate 40 mL vial was charged with nickel(II) chloride glyme (50.3 mg) and 4,4′-di-tert-butyl-2,2′-bipyridine (61.5 mg). 1,2-Dimethoxyethane (22.9 mL) was added and the mixture was sonicated until homogeneous (˜15 min). 10.9 mL of this solution was added to the vial containing the other reaction components. The reaction mixture was degassed via sparging with N₂ for 10 minutes. 4-bromo-2-methylbut-1-ene (0.390 mL, 3.27 mmol) was added before the vial was sealed with parafilm. The vial was then placed in a second-generation Merck photoreactor (50% LED power, 1000 rpm stirring, 10200 rpm fan cooling). After 1 hour, the vial was opened and the mixture was allowed to stir under air. The mixture was filtered, washing with dichloromethane. The filtrate was concentrated in vacuo and the residue was chromatographed on silica gel (80 g column) eluting with 0-90% (25% EtOH/EtOAc)/hexanes to afford compound Int-5a. LCMS anal. calcd. for C₁₄H₂₀N₂O₃: 264.15; Found: 265.24 (M+1)⁺.

### Step B Synthesis of Compound Int-5b

A 100 mL round-bottom flask equipped with a magnetic stirring bar was charged with compound Int-5a (406 mg, 1.536 mmol). THF (15.4 mL) and N-bromosuccinimide (547 mg, 3.07 mmol) were added and the mixture was allowed to stir at room temperature. After 15 minutes, the mixture was concentrated in vacuo to afford compound Int-5b, which was used in Step C of example 5 without further purification. LCMS anal. calcd. for C₁₄H₁₈Br₂N₂O₃: 421.97; Found: 423.07 (M+1)⁺.

### Step C—Synthesis of Compound Int-5c

A 40 mL vial containing compound Int-5b was equipped with a magnetic stirring bar. Cesium carbonate (1501 mg, 4.61 mmol) and DMSO (30.7 mL) were added and the mixture was stirred at room temperature. After 16.5 hours, the reaction mixture was diluted with dichloromethane, water, and brine. The aqueous layer was extracted with three portions of dichloromethane. The combined organic layers were dried over Na₂SO₄, filtered, and concentrated. The residue was purified by flash chromatography on silica gel (80 g column) eluting with 0-100% (25% EtOH/EtOAc)/hexanes. The residue was found to contain DMSO, and was therefore taken up in DCM and washed with LiCl. The LiC layer was back extracted once with DCM. The combined organic layers were dried over Na₂SO₄, filtered, and concentrated to afford compound Int-5c. LCMS anal. calcd. for C₁₄H₇BrN₂O₃: 340.04; Found: 341.10 (M+1)⁺.

### Step D—Synthesis of Compound Int-5d

Compound Int-5c was placed under an atmosphere of N₂. THF (21.2 mL) and DMF (4.25 mL) were added and the mixture was cooled to −78° C. with stirring. Lithium bis(trimethylsilyl)amide 1.0 M in THF (3.825 mL, 3.82 mmol) was added dropwise and the mixture was allowed to stir for 10 minutes at −78° C. 3-Phenyl-2-(phenylsulfonyl)-1,2-oxaziridine (733 mg, 2.80 mmol) was then added dropwise as a solution in a minimum volume of THF. The mixture was allowed to warm to room temperature. After the reaction had warmed to room temperature (˜20 minutes), the mixture was diluted with MeOH and concentrated. The mixture was partitioned between EtOAc and water and the layers were separated. The EtOAc layer was extracted with an additional portion of water. The combined aqueous layers were filtered and purified directly via reverse-phase HPLC eluting with 0-50% (MeCN/H₂O)+0.1% TFA to afford compound Int-5d. LCMS anal. calcd. for C₁₄H₇BrN₂O₄: 356.04; Found: 357.13 (M+1)⁺.

### Step E—Synthesis of Compound Int-5e

A 100 mL round-bottom flask containing compound Int-5d (320 mg, 0.896 mmol) was equipped with a magnetic stirring bar. DMSO (17.9 mL) was added. The flask was evacuated and backfilled with N₂ before (2,4-difluorophenyl)methanamine (319 μl, 2.69 mmol), N,N-diisopropylethylamine (782 μl, 4.48 mmol), and Pd(dppf)Cl₂ (131 mg, 0.179 mmol) were added. The flask was evacuated and backfilled with CO from a balloon three times before being heated to 100° C. and stirred for 24 hours. The mixture was cooled, diluted with a small amount of methanol, and filtered. The resulting solution was purified via reverse-phase HPLC eluting with 20-90% (MeCN/H₂O)+0.1% TFA. Material from product fractions were further purified via chromatography on silica gel (40 g column) eluting with 0-70% (25% EtOH/EtOAc)/hexanes to afford the product that was further purified by chiral preparative SFC (ChiralPak AD-H, 20×250 mm column, 50 mL/min, 100 bar, 50% MeOH/CO₂) to afford Isomer A of compound Int-5e (1^{st} eluting component), Isomer B of compound Int-5e (2^{nd} eluting component), Isomer C of compound Int-5e (3^{rd} eluting component), and Isomer D of compound Int-5e (4^{th} eluting component). Isomer B was further purified under the same SFC conditions to afford material of sufficient purity. LCMS anal. calcd. for C₂₂H₂₃F₂N₃O₅: 447.16; Found: 448.26 (M+1)⁺.

### Step F—Synthesis of Compound 9, Compound 10, Compound 11, and Compound 12

A 4 mL vial equipped with a magnetic stirring bar was charged with Isomer A of compound Int-5e and DMF (223 μl). Lithium chloride (9.47 mg, 0.223 mmol) was added and the mixture was heated to 100° C. with stirring. After 2 hours, the mixture was diluted with DMF and purified directly by reverse-phase HPLC eluting with 5-95% (MeCN/H₂O)+0.1% TFA. Pure fractions were lyophilized to afford compound 9. ¹H NMR (500 MHz, CDCl₃) δ 11.29 (s, 1H); 7.35 (q, J=8.4 Hz, 1H); 6.87-6.78 (m, 2H); 5.73 (t, J=7.9 Hz, 1H); 4.69 (dd, J=15.2, 5.7 Hz, 1H); 4.62 (dd, J=15.0, 5.3 Hz, 1H); 3.88 (d, J=12.9 Hz, 1H); 3.73 (dq, J=14.4, 7.4 Hz, 1H); 3.64-3.49 (m, 2H); 2.81 (dd, J=12.6, 7.4 Hz, 1H); 2.20 (dd, J=12.5, 8.7 Hz, 1H); 1.45 (s, 3H); 1.28 (t, J=7.2 Hz, 3H). LCMS anal. calcd. for C₂₁H₂F₂N₃O₅: 433.14; Found: 434.26 (M+1)⁺.

Following essentially the method employed to produce compound 9 in step F of example 5, compound 10 was prepared from Isomer B of compound Int-5e. ¹H NMR (500 MHz, CDCl₃) δ 10.86 (s, 1H); 7.36 (q, J=8.2 Hz, 1H); 6.89-6.76 (m, 2H); 5.72 (d, J=7.5 Hz, 1H); 4.67 (dd, J 15.2, 5.8 Hz, 1H); 4.60 (dd, J=15.3, 5.5 Hz, 1H); 3.81-3.70 (m, 2H); 3.61-3.49 (m, 2H); 2.43 (d, J=13.5 Hz, 1H); 2.34 (dd, J=13.6, 7.7 Hz, 1H); 1.69 (s, 3H); 1.28 (t, J=7.2 Hz, 3H). LCMS anal. calcd. for C₂₁H₂₁F₂N₃O₅: 433.14; Found: 434.24 (M+1)⁺.

Following essentially the method employed to produce compound 9 in step F of example 5, compound 11 was prepared from Isomer C of compound Int-5e. ¹H NMR (600 MHz, CDCl₃) δ 10.92 (s, 1H); 7.40-7.33 (m, 1H); 6.87-6.78 (m, 2H); 5.68 (d, J=7.5 Hz, 1H); 5.26 (s, 1H); 4.67 (dd, J=15.2, 6.3 Hz, 1H); 4.60 (dd, J=15.4, 5.5 Hz, 1H); 3.81-3.67 (m, 2H); 3.60-3.46 (m, 2H); 2.40 (d, J=13.5 Hz, 1H); 2.33 (dd, J=13.5, 7.8 Hz, 1H); 1.67 (s, 3H); 1.27 (t, J=7.2 Hz, 3H). LCMS anal. calcd. for C₂₁H₂F₂N₃O₅: 433.14; Found: 434.24 (M+1)⁺.

Following essentially the method employed to produce compound 9 in step F of example 5, compound 12 was prepared from Isomer D of compound Int-5e. ¹H NMR (500 MHz, CDCl₃) δ 11.31 (t, J=5.1 Hz, 1H); 7.36 (q, J=8.2 Hz, 1H); 6.87-6.78 (m, 2H); 5.72 (t, J=8.0 Hz, 1H); 4.69 (dd, J=15.3, 5.7 Hz, 1H); 4.62 (dd, J=15.2, 5.6 Hz, 1H); 3.88 (d, J=12.8 Hz, 1H); 3.73 (dq, J=14.3, 7.2 Hz, 1H); 3.63-3.52 (m, 2H); 2.80 (dd, J=12.6, 7.4 Hz, 1H); 2.20 (dd, J 12.5, 8.7 Hz, 1H); 1.45 (s, 3H); 1.28 (t, J=7.2 Hz, 3H). LCMS anal. calcd. for C₂₁H₂F₂N₃O₅: 433.14; Found: 434.24 (M+1)⁺.

### Example 6

### Preparation of Compound Int-6c

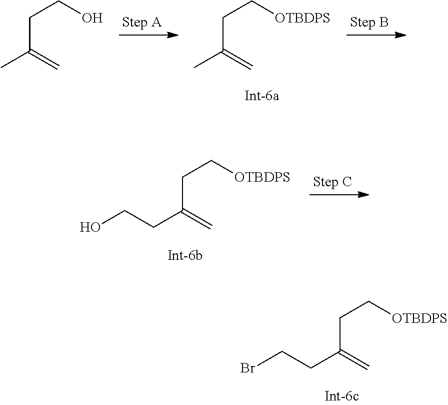

### Step A—Synthesis of Compound Int-6a

To a mixture of 3-methylbut-3-en-1-ol (21 g, 244 mmol) and imidazole (33 g, 487 mmol) in DCM (200 mL) was added TBDPSCl (100 g, 0.365 mmol) in portions. The mixture was stirred at 25° C. for 10 hours before being washed with brine (100 mL). The aqueous layer was extracted with EtOAc (2×100 mL). The combined organic layers were dried over sodium sulfate, filtered, and concentrated in vacuo. The resulting residue was purified by silica gel chromatography eluting with 100% PE to afford compound Int-6a. ¹H NMR (400 MHz, CDCl₃) δ: 7.68-7.66 (m, 4H); 7.42-7.36 (m, 6H); 4.74-4.67 (d, J=24.8 Hz, 2H); 3.77-3.74 (t, J=7.2 Hz, 2H); 2.29-2.25 (t, J=6.8 Hz, 2H); 1.67 (s, 3H); 1.04 (s, 9H).

### Step B—Synthesis of Compound Int-6b

To a mixture of compound Int-6a (10 g, 30.8 mmol) and paraformaldehyde (1.018 g, 33.9 mmol) in DCM (150 mL) was added dropwise dimethylaluminum chloride 1 M in hexanes (40.1 mL, 40.1 mmol) at 0° C. The mixture was stirred at 0° C. for 2 hours before being quenched with water (40 mL). 1 N HCl was added dropwise to dissolve the precipitate. The mixture was extracted with DCM (3×40 mL), and the combined organic layers were dried over sodium sulfate, filtered, and concentrated in vacuo. The resulting residue was purified by silica gel chromatography eluting with 10% EtOAc/PE to afford compound Int-6b. ¹H NMR (400 MHz, CDCl₃) δ: 7.68 (dd, J=7.7, 1.5 Hz, 4H); 7.44-7.37 (m, 6H); 4.89 (s, 2H); 3.78 (t, J=6.8 Hz, 2H); 3.67 (t, J=6.4 Hz, 2H); 2.30-2.22 (m, 4H); 1.06-1.05 (m, 9H).

### Step C—Synthesis of Compound Int-6c

To a stirred solution of compound Int-6b (12 g, 33.8 mmol) in DCM (120 mL) was added triphenylphosphine (10.65 g, 40.6 mmol) and CBr₄ (14.59 g, 44.0 mmol). The mixture was stirred at 20° C. for 2.5 hours before being concentrated in vacuo. The resulting residue was purified by silica gel chromatography eluting with 2% EtOAc/PE to afford compound Int-6c. ¹H NMR (400 MHz, CDCl₃) δ: 7.67 (dd, J=7.9, 1.3 Hz, 4H); 7.45-7.37 (m, 6H); 4.89-4.81 (m, 2H); 3.79-3.71 (m, 2H); 3.39 (t, J=7.5 Hz, 2H); 2.54 (t, J=7.5 Hz, 2H); 2.28 (t, J=6.8 Hz, 2H); 1.07-1.05 (m, 9H).

### Example 7

### Preparation of Compounds 13-16

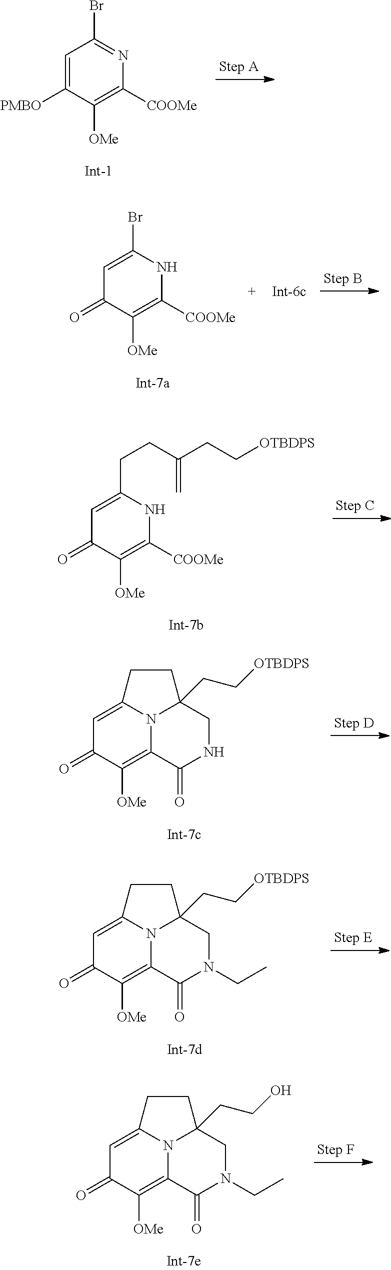

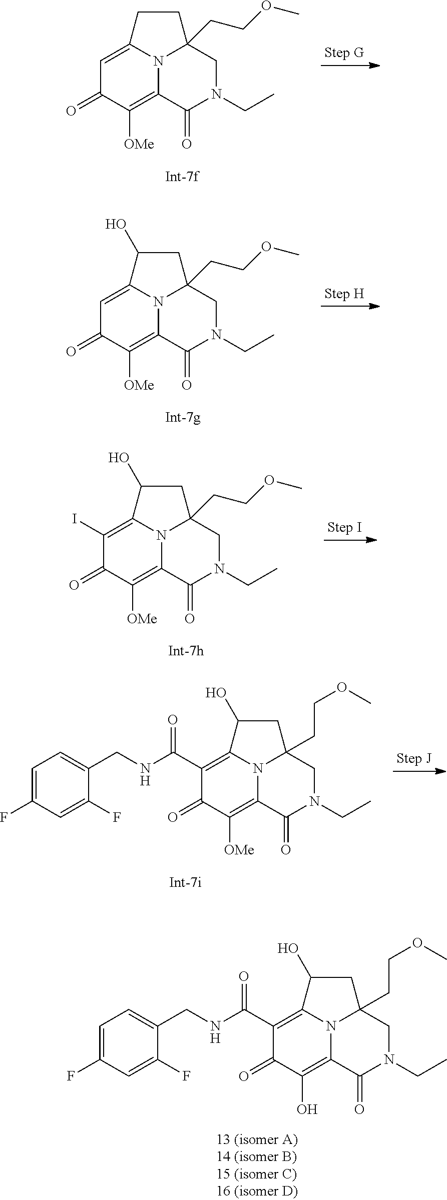

### Step A—Synthesis of Compound Int-7a

To a stirred solution of compound Int-1 (3 g, 7.85 mmol) in DCM (30 mL) was added TFA (10 mL, 130 mmol) dropwise. The mixture was stirred at 25° C. for 3 hours before being concentrated in vacuo. The resulting residue was purified by flash silica gel chromatography (24 g column) eluting with 5% MeOH/DCM to afford compound Int-7a. LCMS anal. calcd. for C₈H₈BrNO₄: 263.0; Found: 263.9 (M+1)⁺.

### Step B—Synthesis of Compound Int-7b

A vial equipped with a magnetic stirring bar (vial A) was charged with Ir[dF(CF₃)ppy]2(dtbpy)PF₆ (4.28 mg, 3.82 μmol), compound Int-7a (100 mg, 0.382 mmol), sodium carbonate (81 mg, 0.763 mmol), and tris(trimethylsilyl)silane (285 mg, 1.145 mmol). Meanwhile, a separate vial (vial B) was charged with nickel (II) chloride ethylene glycol dimethyl ether complex (37 mg, 0.168 mmol) and 4,4′-di-tert-butyl-2,2′-bipyridine (45 mg, 0.168 mmol). DME (16 mL) was added and the mixture was sonicated until homogeneous (˜15 minutes). 3.6 mL of this stock solution was added to vial A containing the other reaction components. The reaction mixture was degassed via sparging with N₂ for 10 minutes. Compound Int-6c (319 mg, 0.763 mmol) was added before the vial was sealed with parafilm. The vial was then placed in front of a Kessil 34 W blue LED lamp. The reaction was allowed to stir with irradiation and cooling by fan. After 16 hours, the reaction mixture was diluted with EtOAc (20 mL) and washed with water (10 mL). The aqueous layer was extracted with EtOAc (3×20 mL). The combined organic layers were dried over sodium sulfate, filtered, and concentrated in vacuo. The residue was purified by preparative TLC plate eluting with 9% MeOH/DCM to afford compound Int-7b. LCMS anal. calcd. for C₃₀H₃₇NO₅Si: 519.2; Found: 520.2 (M+1)⁺.

### Step C—Synthesis of Compound Int-7c

A mixture of compound Int-7b (1.1 g, 2.117 mmol), 0-(2,4-dinitrophenyl)hydroxylamine (0.843 g, 4.23 mmol), and bis[rhodium(α,α,α′,α′-tetramethyl-1,3-benzenedipropionic acid)] (0.032 g, 0.042 mmol) in CF₃CH₂OH (15 mL) was degassed and purged with nitrogen. The resulting mixture was stirred at 50° C. for 10 hours before being concentrated in vacuo and purified by silica gel chromatography eluting with 7% MeOH/DCM to afford compound Int-7c. LCMS anal. calcd. for C₂₉H₃₄N₂O₄Si: 502.2; Found: 503.0 (M+1)⁺.

### Step D—Synthesis of Compound Int-7d

To a mixture of compound Int-7c (1.1 g, 2.188 mmol) and iodoethane (1.024 g, 6.56 mmol) in DMF (15 mL) was added sodium hydride (0.175 g, 4.38 mmol, 60% w/w). The mixture was stirred at 0° C. for 1 hour before being quenched with aqueous 1 M HCl (3 mL, 3 mmol) and concentrated in vacuo. The residue was purified by silica gel chromatography eluting with 9% MeOH/DCM to afford compound Int-7d. LCMS anal. calcd. for C₃₁H₃N₂O₄Si: 530.3; Found: 531.1 (M+1)⁺.

### Step E—Synthesis of Compound Int-7e

To a mixture of compound Int-7d (0.9 g, 1.696 mmol) in THF (5 mL) was added TBAF 1.0 M in THF (2.035 mL, 2.035 mmol). The mixture was stirred at 15° C. for 2 hours before being concentrated in vacuo. The resulting residue was purified by preparative TLC eluting with 30% MeOH/THF to afford compound Int-7e. LCMS anal. calcd. for C₁₅H₂₀N₂O₄: 292.1; Found: 293.0 (M+1)⁺.

### Step F—Synthesis of Compound Int-7f

To a stirred mixture of compound Int-7e (150 mg, 0.513 mmol) in DMF (5 mL) was added sodium hydride (41.0 mg, 1.026 mmol, 60% w/w) and iodomethane (87 mg, 0.616 mmol) at 0° C. The mixture was stirred at 20° C. for 1 hour before being quenched with saturated aqueous NH₄Cl (5 mL) and concentrated in vacuo. The resulting residue was purified by silica gel chromatography eluting with 9% MeOH/DCM to afford compound Int-7f. LCMS anal. calcd. for C₁₆H₂₂N₂O₄: 306.2; Found: 307.0 (M+1)⁺.

### Step G—Synthesis of Compound Int-7g

To a solution of compound Int-7f (100 mg, 0.326 mmol) in THF (8 mL) and DMF (1.5 mL) was added LiHMDS 1 M in THF (0.979 mL, 0.979 mmol) at −78° C. After 20 minutes, to the above mixture was added 3-phenyl-2-(phenylsulfonyl)-1,2-oxaziridine (171 mg, 0.653 mmol) in THF (1 mL) at −78° C. The resulting mixture was stirred at 16° C. for 20 minutes before being quenched with MeOH and concentrated in vacuo. The crude product was purified by preparative TLC eluting with 10% methanol/dichloromethane to afford compound Int-7g. LCMS anal. calcd. for C₁₆H₂₂N₂O₅: 322.2; Found: 323.2 (M+1)⁺.

### Step H—Synthesis of Compound Int-7h

To a stirred solution of compound Int-7g (40 mg, 0.124 mmol) in MeOH (1 mL) was added m-CPBA (26.8 mg, 0.124 mmol) and NIS (55.8 mg, 0.248 mmol). The mixture was stirred at 60° C. for 1 hour before being quenched with saturated aqueous Na₂SO₃ (2 mL) and concentrated in vacuo. The resulting residue was purified by preparative TLC plate eluting with 6% MeOH/DCM to afford compound Int-7h. LCMS anal. calcd. for C₁₆H₂IN₂O₅: 448.1; Found: 449.1 (M+1)⁺.

### Step I—Synthesis of Compound Int-7i

To a stirred solution of compound Int-7h (45 mg, 0.100 mmol) in DMSO (2 mL) was added (2,4-difluorophenyl)methanamine (28.7 mg, 0.201 mmol), N-ethyl-N-isopropylpropan-2-amine (64.9 mg, 0.502 mmol), and Pd(PPh₃)₄ (116 mg, 0.100 mmol) under N₂. The reaction mixture was stirred at 75° C. under CO (15 psi) for 1.5 hours before being treated with water (5 mL) and EtOAc (5 mL). The organic layer was separated, and the aqueous layer was extracted with EtOAc (2×5 mL). The combined organic extracts were washed with water and brine, dried over Na₂SO₄, and concentrated in vacuo. The crude product was purified using preparative TLC (SiO₂, petroleum ether/EtOAc=1:2), which was further purified by SFC (DAICEL CHIRALPAK AD, 10 μm, 30×250 mm column, 60 mL/min, 40% (EtOH+0.1% NH₃H₂O)/CO₂) to afford Isomer A of compound Int-7i (st eluting component), Isomer B of compound Int-7i (2^{nd} eluting component), Isomer C of compound Int-7i (3^{rd} eluting component), and Isomer D of compound Int-7i (4^{th} eluting component). LCMS anal. calcd. for C₂₄H₂₇F₂N₃O₆: 491.2; Found: 492.2 (M+1)⁺.

### Step J—Synthesis of Compound 13, Compound 14, Compound 15, and Compound 16

To a solution of isomer A of compound Int-7i (10 mg, 0.020 mmol) in acetonitrile (1 mL) was added magnesium bromide (18.73 mg, 0.102 mmol). The mixture was stirred at room temperature 20° C. for 1 hour before being filtered and purified by reverse phase HPLC (Boston Green ODS, 5 μm, 30×150 mm column) eluting with 40-70% ACN/(water+0.1% TFA) to afford compound 13. ¹H NMR (400 MHz, CDCl₃) δ:7.46-7.43 (m, 1H); 6.98-6.91 (m, 2H); 5.66-5.62 (t, J=2.4, 1H); 4.64 (s, 2H); 3.90 (s, 2H); 3.72-3.34 (m, 4H); 3.17 (s, 3H); 3.04-2.99 (m, 1H); 2.08-1.93 (m, 3H); 1.28-1.20 (t, J=7.2 Hz, 3H). LCMS anal. calcd. for C₂₃H₂₅F₂N₃O₆: 477.2; Found: 478.0 (M+1)⁺.

Following essentially the method employed to produce compound 13 in step J of example 7, compound 14 was prepared from Isomer B of compound Int-7i. ¹H NMR (400 MHz, CDCl₃) δ: 7.44 (s, 1H); 6.95-6.93 (m, 2H); 5.71-5.69 (m, 1H); 4.62 (s, 2H); 3.98-3.58 (m, 6H); 3.27 (s, 3H); 2.55-2.15 (m, 4H); 1.26-1.20 (t, J=7.2 Hz, 3H). LCMS anal. calcd. for C₂₃H₂₅F₂N₃O₆: 477.2; Found: 478.0 (M+1)⁺.

Following essentially the method employed to produce compound 13 in step J of example 7, compound 15 was prepared from Isomer C of compound Int-7i. ¹H NMR (400 MHz, CD₃Cl) δ: 7.48-7.44 (m, 1H); 6.99-6.91 (m, 2H); 5.70-5.68 (m, 1H); 4.62 (m, 2H); 3.98-3.49 (m, 6H); 3.27 (s, 3H); 2.54-2.13 (m, 4H); 1.26-1.22 (t, J=7.2 Hz, 3H). LCMS anal. calcd. for C₂₃H₂₅F₂N₃O₆: 477.2; Found: 478.0 (M+1)⁺.

Following essentially the method employed to produce compound 13 in step J of example 7, compound 16 was prepared from Isomer D of compound Int-7i. ¹H NMR (400 MHz, CDCl₃) δ: 7.43 (s, 1H); 6.93 (s, 2H); 5.66-5.62 (t, J=2.4, 1H); 4.64 (s, 2H); 3.90 (s, 2H); 3.70-3.34 (m, 4H); 3.17 (s, 3H); 3.04-2.99 (m, 1H); 2.08-1.93 (m, 3H); 1.28-1.22 (t, J=7.2 Hz, 3H). LCMS anal. calcd. for C₂₃H₂₅F₂N₃O₆: 477.2; Found: 478.0 (M+1)⁺.

### Example 8

### Preparation of Compounds 17-20

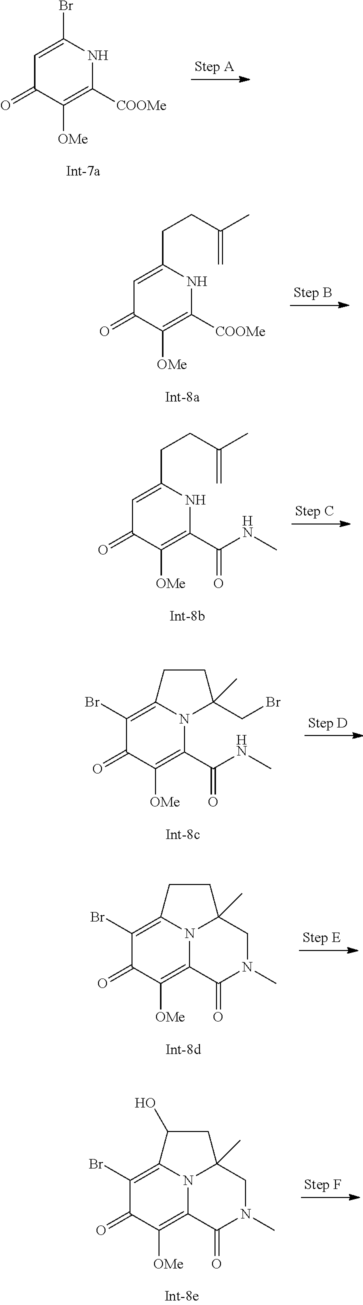

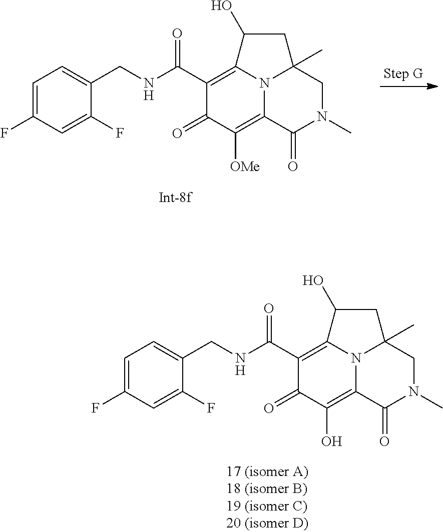

### Step A—Synthesis of Compound Int-8a

A 40 mL vial equipped with a magnetic stirring bar was charged with Ir[dF(CF₃)ppy]2(dtbpy)PF₆ (12.84 mg, 0.011 mmol), compound Int-7a (300 mg, 1.145 mmol), 1,1,1,3,3,3-hexamethyl-2-(trimethylsilyl)trisilane (1.060 mL, 3.43 mmol), and 2,6-lutidine (0.265 mL, 2.290 mmol). Meanwhile, a separate 40 mL vial was charged with nickel(II) chloride glyme (37.0 mg) and 4,4′-di-tert-butyl-2,2′-bipyridine (45.0 mg). 1,2-Dimethoxyethane (16.7 mL) was added and the mixture was sonicated until homogeneous (˜15 minutes). 11.4 mL of this stock solution was added to the vial containing the other reaction components. The reaction mixture was degassed via sparging with N₂ for 10 minutes. 4-bromo-2-methylbut-1-ene (0.409 mL, 3.43 mmol) was added before the vial was sealed with parafilm. The vial was then placed in a second-generation Merck photoreactor (50% LED power, 700 rpm stirring, 10200 rpm fan cooling). After 2 hours, the reaction was removed from the light and concentrated. The residue was purified by flash chromatography on silica gel (120 g column) eluting with 0-100% (25% EtOH/EtOAc)/hexanes to afford impure product. This material was further purified by reverse-phase HPLC (Sunfire Prep C18 OBD, 10 μm, 50×250 mm column) eluting with 0-80% (MeCN/H₂O)+0.1% TFA to afford compound Int-8a. LCMS anal. calcd. for C₁₃H₇NO₄: 251.12; Found: 252.15 (M+1)⁺.

### Step B—Synthesis of Compound Int-8b

A 40 mL vial equipped with a magnetic stirring bar was charged with a solution of compound Int-8a (505 mg, 2.010 mmol) in methanol (10.0 mL). Methylamine 2.0 M in THF (10.05 mL, 20.10 mmol) was added and the mixture was allowed to stir at room temperature. After 6 hours, the mixture was concentrated in vacuo to afford compound Int-8b, which was used in Step C of example 8 without further purification. LCMS anal. calcd. for C₁₃H₁₈N₂O₃: 250.13; Found: 251.16 (M+1)⁺.

### Step C—Synthesis of Compound Int-8c

A 40 mL vial containing compound Int-8b (503 mg, 2.01 mmol) was equipped with a magnetic stirring bar. THF (20.1 mL) and N-bromosuccinimide (787 mg, 4.42 mmol) were added and the mixture was stirred at room temperature. After 15 minutes, the mixture was diluted with MeOH and concentrated to afford compound Int-8c, which was used in Step D of example 8 without further purification. LCMS anal. calcd. for C₁₃H₁₆Br₂N₂O₃: 407.95; Found: 409.04 (M+1)⁺.

### Step D—Synthesis of Compound Int-8d

A 40 mL vial equipped with a magnetic stirring bar was charged with compound Int-8c (820 mg, 2.01 mmol). DMSO (20.1 mL) and cesium carbonate (2619 mg, 8.04 mmol) were added and the mixture was allowed to stir at room temperature. After 1 hour, the mixture was filtered, washed with DMSO, and purified by reverse-phase HPLC (Sunfire Prep C18 OBD, 10 μm, 50×250 mm) eluting with 0-60% (MeCN/H₂O)+0.1% TFA. The solids from the filtration were taken up in MeOH and filtered again. The filtrate was concentrated and taken up in DMSO/MeOH. This mixture was purified by reverse-phase HPLC using the same conditions described above. Product fractions were concentrated in vacuo to afford compound Int-8d. LCMS anal. calcd. for C₁₃H₁₅BrN₂O₃: 326.03; Found: 327.08 (M+1)⁺.

### Step E—Synthesis of Compound Int-8e

A flame-dried 200 mL round-bottom flask equipped with a magnetic stirring bar was charged with compound Int-8d (608 mg, 1.858 mmol) and placed under an atmosphere of N₂. THF (24.8 mL) and DMF (12.4 mL) were added and the mixture was cooled to −78° C. with stirring. Lithium bis(trimethylsilyl)amide 1.0 M in THF (5.575 mL, 5.58 mmol) was added dropwise and the mixture was allowed to stir for 10 minutes at −78° C. 3-phenyl-2-(phenylsulfonyl)-1,2-oxaziridine (1068 mg, 4.09 mmol) was then added dropwise as a solution in a minimum volume of THF. The mixture was allowed to warm to room temperature. The mixture was partitioned between water and EtOAc. A small amount of brine was added to facilitate separation of the layers. The EtOAc layer was washed with two additional small portions of water. The combined aqueous layers were filtered and directly purified by reverse-phase HPLC (Sunfire Prep C18 OBD, 10 μm, 50×250 mm column) eluting with an initial 100% H₂O+0.1% TFA isocratic hold, followed by 0-50% (MeCN/H₂O)+0.1% TFA. The product was collected and further purified by reverse-phase HPLC (Phenomenex Luna Prep C18, 5 μm, 50×250 mm column) eluting with an initial 100% H₂O+0.1% TFA isocratic hold followed, by 0-50% (MeCN/H₂O)+0.1% TFA. Product fractions were concentrated to afford compound Int-8e. LCMS anal. calcd. for C₁₃H₁₅BrN₂O₄: 342.02; Found: 343.07 (M+1)⁺.

### Step F—Synthesis of Compound Int-8f

A 50 mL round-bottom flask equipped with a magnetic stirring bar was charged with compound Int-8e (140 mg, 0.408 mmol). DMSO (8.16 mL), (2,4-difluorophenyl)methanamine (0.145 mL, 1.224 mmol), N,N-diisopropylethylamine (0.356 mL, 2.040 mmol), and Pd(dppf)Cl2 (59.7 mg, 0.082 mmol) were added. The flask was evacuated and backfilled with CO from a balloon three times, then heated to 100° C. and stirred for 7.5 hours. The reaction was cooled to room temperature, filtered, and purified by reverse-phase HPLC (Sunfire Prep C18 OBD, 10 μm, 50×250 mm column) eluting with 5-85% (MeCN/H₂O)+0.1% TFA to afford a pale yellow foam/solid. This material was further purified by chiral preparative SFC (ChiralPak IA, 20×150 mm column, 65 mL/min, 100 bar, 20-35% ethanol/CO₂) to afford Isomer A of compound Int-8f (1^{st} eluting component), Isomer B of compound Int-8f (2^{nd} eluting component), Isomer C of compound Int-8f (3^{rd} eluting component), and Isomer D of compound Int-8f (4^{th} eluting component). LCMS anal. calcd. for C₂₁H₂₁F₂N₃O₅: 433.14; Found: 434.21 (M+1)⁺.

### Step G—Synthesis of Compound 17, Compound 18, Compound 19, and Compound 20

A 20 mL vial containing Isomer A of compound Int-8f (21 mg, 0.048 mmol) was equipped with a magnetic stirring bar. DMF (0.500 mL) was added followed by lithium chloride (20.54 mg, 0.485 mmol) and the mixture was heated to 100° C. with stirring. After 3 hours, the reaction was cooled to room temperature. The mixture was diluted with DMSO and purified by reverse-phase HPLC (Sunfire Prep C18 OBD, 5 μm, 30×150 mm column) eluting with 5-95% (MeCN/H₂O)+0.1% TFA. Product fractions were concentrated, co-evaporated with DCM/MeOH/toluene, and lyophilized to afford compound 17. ¹H NMR (500 MHz, SO(CD₃)₂) δ 11.66 (s, 1H); 11.45 (t, J=5.5 Hz, 1H); 7.44 (q, J=7.9, 7.4 Hz, 1H); 7.30-7.21 (m, 1H); 7.13-7.04 (m, 1H); 6.89 (s, 1H); 5.63 (t, J=7.9 Hz, 1H); 4.58 (qd, J=14.9, 6.1 Hz, 2H); 3.95 (d, J 12.9 Hz, 1H); 3.73 (d, J=12.8 Hz, 1H); 3.09 (s, 3H); 2.71 (dd, J=11.9, 7.6 Hz, 1H); 2.03-1.96 (m, 1H); 1.36 (s, 3H). LCMS anal. calcd. for C₂₀H₁₉F₂N₃O₅: 419.13; Found: 420.23 (M+1)⁺.

Following essentially the method employed to produce compound 17 in step G of example 8, compound 18 was prepared from Isomer B of compound Int-8f. ¹H NMR (500 MHz, CDCl₃) δ 11.41 (br. s, 1H); 10.89 (t, J=5.0 Hz, 1H); 7.37 (q, J=8.2 Hz, 1H); 6.88-6.77 (m, 2H); 5.66 (d, J=7.4 Hz, 1H); 5.24 (s, 1H); 4.70-4.56 (m, 2H); 3.84 (d, J=12.7 Hz, 1H); 3.51 (d, J=12.7 Hz, 1H); 3.21 (s, 3H); 2.39 (d, J=13.4 Hz, 1H); 2.32 (dd, J=13.4, 7.5 Hz, 1H); 1.68 (s, 3H). LCMS anal. calcd. for C₂₀H₁₉F₂N₃O₅: 419.13; Found: 420.23 (M+1)⁺.

Following essentially the method employed to produce compound 17 in step G of example 8, compound 19 was prepared from Isomer C of compound Int-8f. ¹H NMR (500 MHz, SO(CD₃)₂) δ 11.66 (s, 1H); 11.45 (t, J=5.8 Hz, 1H); 7.44 (q, J=8.5 Hz, 1H); 7.26 (td, J 10.6, 2.4 Hz, 1H); 7.08 (td, J=8.8, 2.2 Hz, 1H); 6.89 (s, 1H); 5.62 (t, J=8.0 Hz, 1H); 4.58 (qd, J 15.1, 5.9 Hz, 2H); 3.95 (d, J=12.8 Hz, 1H); 3.73 (d, J=12.8 Hz, 1H); 3.08 (s, 3H); 2.71 (dd, J=12.2, 7.4 Hz, 1H); 1.99 (dd, J=12.1, 8.9 Hz, 1H); 1.36 (s, 3H). LCMS anal. calcd. for C₂₀H₁₉F₂N₃O₅: 419.13; Found: 420.22 (M+1)⁺.

Following essentially the method employed to produce compound 17 in step G of example 8, compound 20 was prepared from Isomer D of compound Int-8f. ¹H NMR (500 MHz, SO(CD₃)₂) δ 11.47 (s, 1H); 10.85 (t, J=5.3 Hz, 1H); 7.42 (q, J=8.4 Hz, 1H); 7.24 (td, J 10.5, 10.0, 2.2 Hz, 1H); 7.07 (td, J=8.6, 1.9 Hz, 1H); 5.68 (d, J=7.1 Hz, 1H); 5.45 (s, 1H); 4.56 (d, J=5.4 Hz, 2H); 3.79 (d, J=12.7 Hz, 1H); 3.72 (d, J=12.8 Hz, 1H); 3.09 (s, 3H); 2.32 (dd, J=13.2, 7.3 Hz, 1H); 2.15 (d, J=13.4 Hz, 1H); 1.56 (s, 3H). LCMS anal. calcd. for C₂₀H₁₉F₂N₃O₅: 419.13; Found: 420.22 (M+1)⁺.

### Example 9

### Preparation of Compounds 21-24

Starting from Int-8e, using essentially the same method described in Step F and Step G in example 8 with the exception of substituting with 4-fluorobenzylamine, purifying by chiral preparative SFC (ChiralPak AD-H, 20×250 mm column, 60 mL/min, 100 bar, 35% MeOH/CO₂) to afford a mixture of Isomers A and Isomer B, Isomer C, and Isomer D, and further purifying the mixture of Isomer A and Isomer B by chiral preparative SFC (ChiralPak AD-H, 20×250 mm column, 70 mL/min, 100 bar, 25% MeOH/CO₂) to afford Isomer A and Isomer B in Step F, the following compounds were prepared:

| Compound # | Structure | MS (M + H)⁺ | ¹H NMR |
|---|---|---|---|
| 21 (Isomer A) | 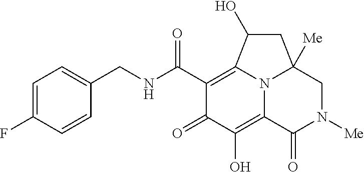 [SMILES: CN1CC2(C)CC(O)c3c(C(=O)NCc4ccc(F)cc4)c(=O)c(O)c(n32)C1=O] | 402.29 | (600 MHz, SO(CD₃)₂) δ 11.67 (s, 1H); 11.46 (t, J = 5.5 Hz, 1H); 7.37 (dd, J = 8.2, 5.5 Hz, 2H); 7.18 (t, J = 8.9 Hz, 2H); 6.98 (s, 1H); 5.63 (t, J = 8.6 Hz, 1H); 4.58 (dd, J = 15.1, 5.7 Hz, 1H); 4.53 (dd, J = 14.3, 5.7 Hz, 1H); 3.95 (d, J = 12.9 Hz, 1H); 3.74 (d, J = 12.8 Hz, 1H); 3.08 (s, 3H); 2.71 (dd, J = 12.1, 7.3 Hz, 1H); 2.05-1.95 (m, 1H); 1.36 (s, 3H). |
| 22 (Isomer B) | 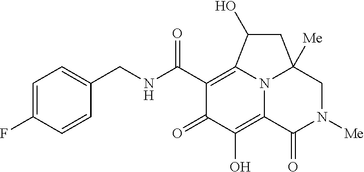 [SMILES: CN1CC2(C)CC(O)c3c(C(=O)NCc4ccc(F)cc4)c(=O)c(O)c(n32)C1=O] | 402.30 | (600 MHz, SO(CD₃)₂) δ 11.49 (s, 1H); 10.85 (t, J = 5.9 Hz, 1H); 7.37 (dd, J = 8.4, 5.8 Hz, 2H); 7.17 (t, J = 8.8 Hz, 2H); 5.69 (d, J = 7.2 Hz, 1H); 5.49 (s, 1H); 4.54 (d, J = 5.8 Hz, 2H); 3.80 (d, J = 12.7 Hz, 1H); 3.73 (d, J = 12.8 Hz, 1H); 3.09 (s, 3H); 2.33 (dd, J = 13.2, 7.4 Hz, 1H); 2.16 (d, J = 13.4 Hz, 1H); 1.56 (s, 3H). |
| 23 (Isomer C) | 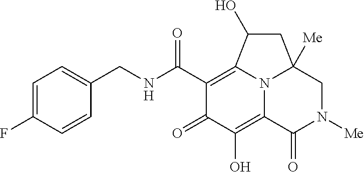 [SMILES: CN1CC2(C)CC(O)c3c(C(=O)NCc4ccc(F)cc4)c(=O)c(O)c(n32)C1=O] | 402.31 | (600 MHz, SO(CD₃)₂) δ 11.67 (s, 1H); 11.46 (t, J = 5.8 Hz, 1H); 7.37 (dd, J = 8.3, 5.8 Hz, 2H); 7.18 (t, J = 8.8 Hz, 2H); 6.98 (s, 1H); 5.63 (t, J = 8.2 Hz, 1H); 4.58 (dd, J = 14.7, 5.5 Hz, 1H); 4.53 (dd, J = 15.1, 5.5 Hz, 1H); 3.95 (d, J = 12.8 Hz, 1H); 3.74 (d, J = 12.7 Hz, 1H); 3.08 (s, 3H); 2.71 (dd, J = 12.2, 7.3 Hz, 1H); 2.00 (dd, J = 12.3, 8.8 Hz, 1H); 1.36 (s, 3H). |
| 24 (Isomer D) | 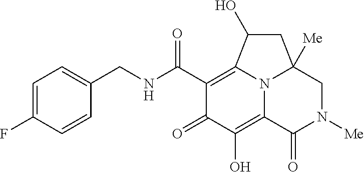 [SMILES: CN1CC2(C)CC(O)c3c(C(=O)NCc4ccc(F)cc4)c(=O)c(O)c(n32)C1=O] | 402.31 | (600 MHz, SO(CD₃)₂) δ 11.49 (s, 1H); 10.85 (t, J = 5.6 Hz, 1H); 7.37 (dd, J = 8.3, 5.7 Hz, 2H); 7.17 (t, J = 8.8 Hz, 2H); 5.69 (dd, J = 7.2, 2.7 Hz, 1H); 5.50 (d, J = 3.0 Hz, 1H); 4.54 (d, J = 5.9 Hz, 2H); 3.80 (d, J = 12.6 Hz, 1H); 3.73 (d, J = 12.8 Hz, 1H); 3.09 (s, 3H); 2.33 (dd, J = 13.4, 7.4 Hz, 1H); 2.16 (d, J = 13.3 Hz, 1H); 1.56 (s, 3H). |

### Example 10

### Preparation of Compounds 25-28

Starting from Int-8e, using essentially the same method described in Step F and Step G in example 8 with the exception of substituting with 2,4,6-trifluorobenzylamine, purifying by chiral preparative SFC (ChiralPak AD-H, 20×150 mm column, 60 m/m, 100 bar, 20 MeOH/CO₂) to afford Isomer A, Isomer B, Isomer C, and Isomer D, and further purifying Isomer C and Isomer under the same SFC conditions to afford material of sufficient purity in Step F, the following compounds were prepared:

| Compound # | Structure | MS (M + H)⁺ | ¹H NMR |
|---|---|---|---|
| 25 (Isomer A) | 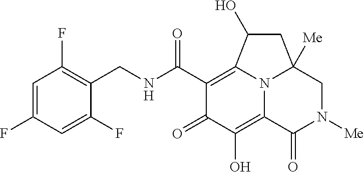 [SMILES: CN1CC2(C)CC(O)c3c(C(=O)NCc4c(F)cc(F)cc4F)c(=O)c(O)c(n32)C1=O] | 438.22 | (600 MHz, CDCl₃) δ 11.34 (t, J = 5.5 Hz, 1H); 7.04 (s, 1H); 6.67 (t, J = 8.0 Hz, 2H); 5.70 (t, J = 7.8 Hz, 1H); 4.71 (dd, J = 14.4, 5.6 Hz, 1H); 4.63 (dd, J = 14.4, 5.4 Hz, 1H); 3.89 (d, J = 12.7 Hz, 1H); 3.52 (d, J = 12.8 Hz, 1H); 3.21 (s, 3H); 2.77 (dd, J = 12.6, 7.3 Hz, 1H); 2.15 (dd, J = 12.6, 8.6 Hz, 1H); 1.44 (s, 3H). |
| 26 (Isomer B) | 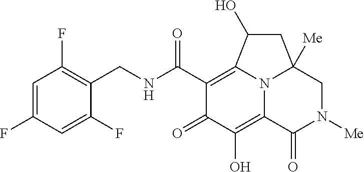 [SMILES: CN1CC2(C)CC(O)c3c(C(=O)NCc4c(F)cc(F)cc4F)c(=O)c(O)c(n32)C1=O] | 438.21 | (500 MHz, CDCl₃) δ 11.35 (br. s, 1H); 10.83 (s, 1H); 6.67 (t, J = 8.0 Hz, 2H); 5.66 (d, J = 7.2 Hz, 1H); 5.21 (s, 1H); 4.66 (dq, J = 17.1, 9.3, 7.5 Hz, 2H); 3.85 (d, J = 12.7 Hz, 1H); 3.50 (d, J = 12.7 Hz, 1H); 3.20 (s, 3H); 2.38 (d, J = 13.5 Hz, 1H); 2.33 (dd, J = 13.4, 7.3 Hz, 1H); 1.67 (s, 3H). |
| 27 (Isomer C) | 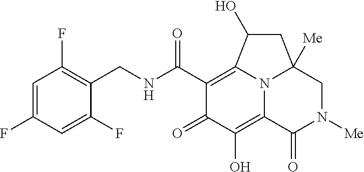 [SMILES: CN1CC2(C)CC(O)c3c(C(=O)NCc4c(F)cc(F)cc4F)c(=O)c(O)c(n32)C1=O] | 438.21 | (500 MHz, CDCl₃) δ 11.38-11.31 (m, 1H); 7.03 (s, 1H); 6.67 (t, J = 8.0 Hz, 2H); 5.70 (t, J = 8.0 Hz, 1H); 4.71 (dd, J = 14.5, 5.7 Hz, 1H); 4.63 (dd, J = 14.6, 5.3 Hz, 1H); 3.89 (d, J = 12.8 Hz, 1H); 3.52 (d, J = 12.8 Hz, 1H); 3.21 (s, 3H); 2.77 (dd, J = 12.6, 7.4 Hz, 1H); 2.15 (dd, J = 12.4, 8.6 Hz, 1H); 1.44 (s, 3H). |
| 28 (Isomer D) | 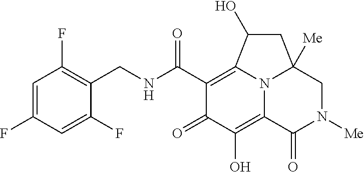 [SMILES: CN1CC2(C)CC(O)c3c(C(=O)NCc4c(F)cc(F)cc4F)c(=O)c(O)c(n32)C1=O] | 438.22 | (500 MHz, CDCl₃) δ 11.35 (br. s, 1H); 10.86 (s, 1H); 6.67 (t, J = 8.1 Hz, 2H); 5.70 (d, J = 7.6 Hz, 1H); 5.23 (s, 1H); 4.73-4.58 (m, 2H); 3.79 (d, J = 12.7 Hz, 1H); 3.52 (d, J = 12.7 Hz, 1H); 3.21 (s, 3H); 2.40 (d, J = 13.6 Hz, 1H); 2.30 (dd, J = 13.5, 7.8 Hz, 1H); 1.68 (s, 3H). |

### Example 11

### Preparation of Compounds 29-32

Starting from Int-8e, using essentially the same method described in Step F and Step G in example 8 with the exception of substituting with 3-chloro-2,6-difluorobenzyamine, purifying by chiral preparative SFC (ChiralPak IA, 20×150 mm column, 65 mL/min, 100 bar, 25% EtOH/CO₂) to afford Isomer A, Isomer B, Isomer C, and Isomer D, and further purifying Isomer B, Isomer C, and Isomer D under the same SFC conditions to afford material of sufficient purity in Step F, the following compounds were prepared:

| Compound # | Structure | MS (M + H)⁺ | ¹H NMR |
|---|---|---|---|
| 29 (Isomer A) | 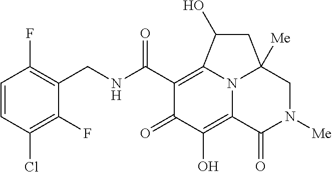 [SMILES: CN1CC2(C)CC(O)c3c(C(=O)NCc4c(F)ccc(Cl)c4F)c(=O)c(O)c(n32)C1=O] | 454.20 | (500 MHz, CDCl₃) δ 11.40 (s, 1H); 7.35-7.27 (m, 1H); 7.01 (s, 1H); 6.87 (t, J = 8.5 Hz, 1H); 5.70 (t, J = 8.0 Hz, 1H); 4.79 (dd, J = 14.6, 5.7 Hz, 1H); 4.69 (dd, J = 14.4, 5.3 Hz, 1H); 3.89 (d, J = 12.7 Hz, 1H); 3.52 (d, J = 12.8 Hz, 1H); 3.21 (s, 3H); 2.77 (dd, J = 12.6, 7.5 Hz, 1H); 2.16 (dd, J = 12.4, 8.7 Hz, 1H); 1.44 (s, 3H). |
| 30 (Isomer B) | 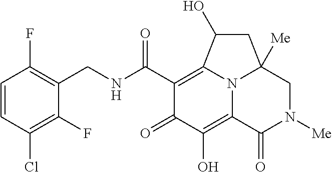 [SMILES: CN1CC2(C)CC(O)c3c(C(=O)NCc4c(F)ccc(Cl)c4F)c(=O)c(O)c(n32)C1=O] | 454.21 | (500 MHz, CDCl₃) δ 10.90 (t, J = 4.6 Hz, 1H); 7.34-7.28 (m, 1H); 6.88 (t, J = 8.7 Hz, 1H); 5.68 (d, J = 7.6 Hz, 1H); 5.21 (s, 1H); 4.72 (d, J = 5.5 Hz, 2H); 3.81 (d, J = 12.7 Hz, 1H); 3.51 (d, J = 12.7 Hz, 1H); 3.21 (s, 3H); 2.40 (d, J = 13.6 Hz, 1H); 2.32 (dd, J = 13.6, 7.7 Hz, 1H); 1.68 (s, 3H). |
| 31 (Isomer C) | 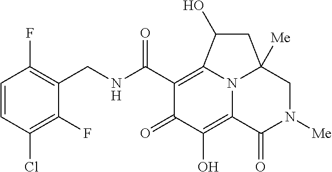 [SMILES: CN1CC2(C)CC(O)c3c(C(=O)NCc4c(F)ccc(Cl)c4F)c(=O)c(O)c(n32)C1=O] | 454.20 | (500 MHz, CDCl₃) δ 10.90 (t, J = 5.2 Hz, 1H); 7.34-7.28 (m, 1H); 6.87 (t, J = 8.7 Hz, 1H); 5.68 (d, J = 7.6 Hz, 1H); 5.20 (s, 1H); 4.72 (d, J = 5.5 Hz, 2H); 3.81 (d, J = 12.7 Hz, 1H); 3.51 (d, J = 12.7 Hz, 1H); 3.21 (s, 3H); 2.40 (d, J = 13.5 Hz, 1H); 2.32 (dd, J = 13.6, 7.6 Hz, 1H); 1.68 (s, 3H). |
| 32 (Isomer D) | 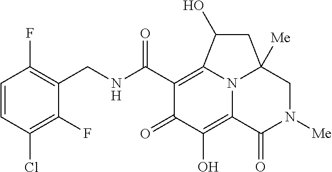 [SMILES: CN1CC2(C)CC(O)c3c(C(=O)NCc4c(F)ccc(Cl)c4F)c(=O)c(O)c(n32)C1=O] | 454.20 | (500 MHz, CDCl₃) δ 11.39 (t, J = 5.8 Hz, 1H); 7.33-7.28 (m, 1H); 7.01 (s, 1H); 6.87 (t, J = 8.8 Hz, 1H); 5.70 (t, J = 7.9 Hz, 1H); 4.79 (dd, J = 14.4, 5.9 Hz, 1H); 4.69 (dd, J = 14.6, 5.4 Hz, 1H); 3.89 (d, J = 12.8 Hz, 1H); 3.52 (d, J = 12.7 Hz, 1H); 3.21 (s, 3H); 2.77 (dd, J = 12.5, 7.4 Hz, 1H); 2.16 (dd, J = 12.6, 8.7 Hz, 1H); 1.44 (s, 3H). |

### Example 12

### Preparation of Compounds 33-36

Starting from Int-8e, using essentially the same method described in Step F and Step G in example 8 with the exception of substituting with 3-chloro-2-fluorobenzylamine and purifying by chiral preparative SFC (ChiralPak IA, 20×150 mm column, 65 mL/min, 100 bar, 2535% MeOH/CO₂) to afford Isomer A, Isomer B, Isomer C, and Isomer Din Step F, the following compounds were prepared:

| Compound # | Structure | MS (M + H)⁺ | ¹H NMR |
|---|---|---|---|
| 33 (Isomer A) | 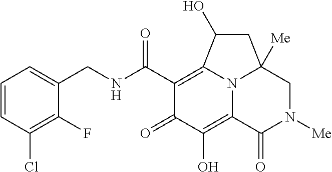 [SMILES: CN1CC2(C)CC(O)c3c(C(=O)NCc4cccc(Cl)c4F)c(=O)c(O)c(n32)C1=O] | 436.20 | (500 MHz, CDCl₃) δ 11.46 (s, 1H); 7.33-7.27 (m, 2H); 7.07-6.98 (m, 2H); 5.70 (t, J = 8.0 Hz, 1H); 4.72 (qd, J = 16.3, 15.6, 5.7 Hz, 2H); 3.90 (d, J = 12.7 Hz, 1H); 3.53 (d, J = 12.8 Hz, 1H); 3.22 (s, 3H); 2.78 (dd, J = 12.3, 7.1 Hz, 1H); 2.19-2.14 (m, 1H); 1.46 (s, 3H). |
| 34 (Isomer B) | 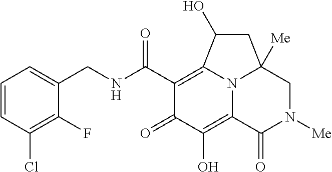 [SMILES: CN1CC2(C)CC(O)c3c(C(=O)NCc4cccc(Cl)c4F)c(=O)c(O)c(n32)C1=O] | 436.18 | (500 MHz, CDCl₃) δ 10.90 (s, 1H); 7.31-7.24 (m, 2H); 7.03 (d, J = 15.8 Hz, 1H); 5.65 (s, 1H); 5.39-5.11 (m, 1H); 4.80-4.49 (m, 2H); 3.81-3.61 (m, 1H); 3.55-3.38 (m, 1H); 3.17 (s, 3H); 2.43-2.32 (m, 1H); 2.31-2.19 (m, 1H); 1.67 (d, J = 13.6 Hz, 3H). |
| 35 (Isomer C) | 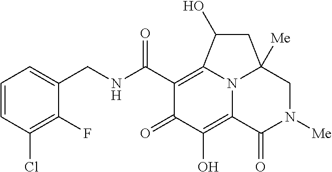 [SMILES: CN1CC2(C)CC(O)c3c(C(=O)NCc4cccc(Cl)c4F)c(=O)c(O)c(n32)C1=O] | 436.19 | (500 MHz, CDCl₃) δ 10.98-10.91 (m, 1H); 7.30 (q, J = 6.6 Hz, 2H); 7.04 (t, J = 7.8 Hz, 1H); 5.67 (d, J = 7.5 Hz, 1H); 5.21 (s, 1H); 4.73 (dd, J = 15.3, 5.6 Hz, 1H); 4.67 (dd, J = 15.7, 5.5 Hz, 1H); 3.82 (d, J = 12.7 HZ, 1H); 3.53 (d, J = 12.7 Hz, 1H); 3.22 (s, 3H); 2.40 (d, J = 13.5 Hz, 1H); 2.32 (dd, J = 13.5, 7.6 Hz, 1H); 1.69 (s, 3H). |
| 36 (Isomer D) | 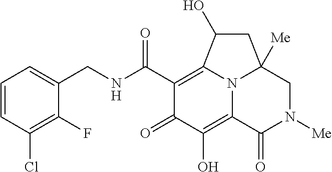 [SMILES: CN1CC2(C)CC(O)c3c(C(=O)NCc4cccc(Cl)c4F)c(=O)c(O)c(n32)C1=O] | 436.17 | (600 MHz, CDCl₃) δ 11.47 (s, 1H); 7.32-7.27 (m, 2H); 7.06-7.00 (m, 2H); 5.69 (t, J = 7.8 Hz, 1H); 4.75 (dd, J = 15.2, 6.1 Hz, 1H); 4.69 (dd, J = 15.5, 6.0 Hz, 1H); 3.90 (d, J = 12.7 Hz, 1H); 3.53 (d, J = 12.7 Hz, 1H); 3.22 (s, 3H); 2.78 (dd, J = 12.6, 7.5 Hz, 1H); 2.16 (dd, J = 12.4, 8.4 Hz, 1H); 1.46 (s, 3H). |

### Example 13

### Preparation of Compound Int-13-13b

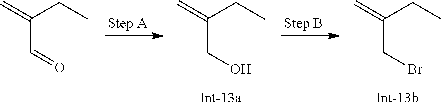

### Step A—Synthesis of Compound Int-13a

To a solution of 2-methylenebutanal (20 g, 238 mmol) in MeOH (120 mL) was added NaBH₄ (9.45 g, 250 mmol) in portions. The mixture was stirred at 0° C. for 1 hour. The mixture was quenched with saturated aqueous NH₄Cl (40 mL) and diluted with water (80 mL) before being extracted with EtOAc (3×100 mL). The combined organic layers were dried over Na₂SO₄, filtered, and evaporated. The resulting residue was purified by flash silica gel chromatography (80 g column) eluting with 0-10% EtOAc/petroleum ether to afford compound Int-13a. ¹H NMR (400 MHz, CDCl₃) δ: 4.99 (s, 1H); 4.98 (s, 1H); 4.07 (s, 2H); 2.08-2.02 (m, 2H); 1.22-1.02 (m, 3H).

### Step B—Synthesis of Compound Int-13b

To a solution of compound Int-13a (2 g, 23.22 mmol) in DCM (40 mL) was added PBr₃ (1.095 mL, 11.61 mmol) at 0° C. The mixture was warmed to 20° C. and stirred for 12 hours. The mixture was cooled to 0° C., quenched with 5% aqueous K₂CO₃ (15 mL), and diluted with water (20 mL). The organic phase was isolated, washed with brine (20 mL), dried over Na₂SO₄, filtered, and concentrated in vacuo to afford compound Int-13b. This material was used in Step D of example 14 without further purification. ¹H NMR (400 MHz, CDCl₃) δ: 5.23 (s, 1H); 4.99 (s, 1H); 3.83 (s, 2H); 2.20-2.14 (m, 2H); 1.03-1.00 (m, 3H).

### Example 14

### Preparation of Compounds 37-40

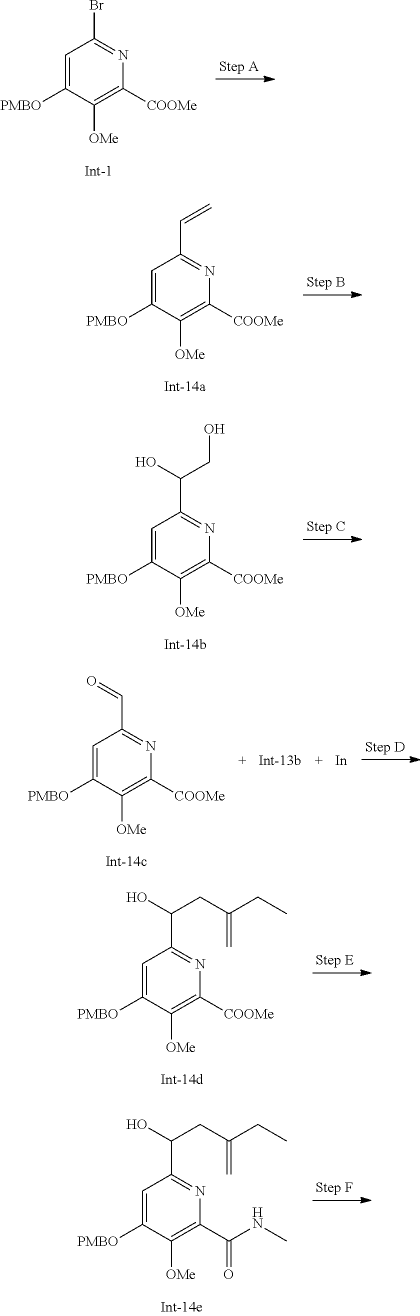

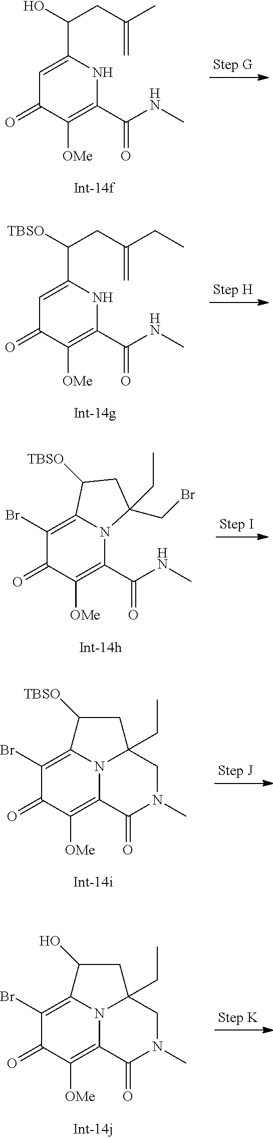

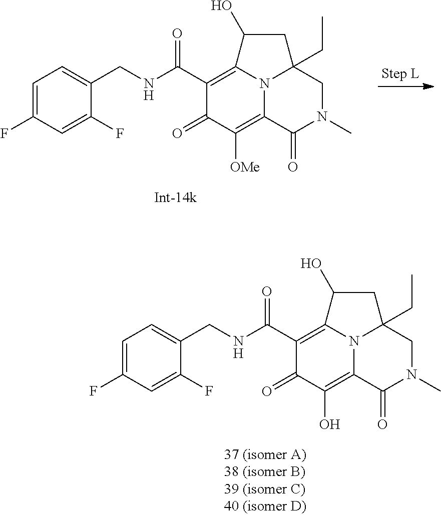

### Step A Synthesis of Compound Int-14a

1,1′-Bis(diphenylphosphino)ferrocene-palladium(II)dichloride dichloromethane complex (590.9 mg, 0.724 mmol) was added to a stirred solution of compound Int-1 (5.04 g, 13.19 mmol), potassium vinyltrifluoroborate (3.52 g, 26.3 mmol), and potassium carbonate (3.65 g, 26.4 mmol) in dioxane (53.0 mL) and water (13.0 mL). The reaction mixture was degassed (3×) and placed under nitrogen before being heated to 80° C. for 3 hours. The reaction mixture was cooled to room temperature before being partitioned between EtOAc (200 mL) and water (200 mL). The aqueous layer was extracted with EtOAc (2×100 mL). The organic layers were combined, washed with brine (1×30 mL), dried over MgSO₄, filtered, and evaporated under reduced pressure. The resulting solid was purified by silica gel column (220 g) chromatography eluting with 0-40% (25% EtOH/EtOAc)/hexanes to afford compound Int-14a. LCMS anal. calcd. for C₁₈H₁₉NO₅: 329.13; Found: 330.21 (M+1)⁺.

### Step B—Synthesis of Compound Int-14b

Osmium tetroxide 2.5 wt % in t-butanol (3.4 mL, 0.271 mmol) and NMO (1.4025 g, 11.97 mmol) were added to a stirred solution of compound Int-14a (1.7072 g, 5.18 mmol) in THF (24.0 mL), t-butanol (21.0 mL), and water (4.0 mL). The reaction mixture was stirred at room temperature for 2.5 hours before being diluted with THF (52.0 mL). Sodium metabisulfite (24.60 g, 129.4 mmol) was added to the reaction mixture, which was stirred for an additional hour before being filtered through a pad of celite. The filtrate was dried over Na₂SO₄, filtered, and evaporated under reduced pressure. The resulting oil was purified by silica gel column (120 g) chromatography eluting with 0-8% MeOH/DCM to afford compound Int-14b. LCMS anal. calcd. for C₁₈H₂₁NO₇: 363.13; Found: 364.25 (M+1)⁺.

### Step C—Synthesis of Compound Int-14c

Sodium periodate (2.1 g, 9.82 mmol) was added to a stirred solution of compound Int-14b (1.7344 g, 4.77 mmol) in THF (38.0 mL) and water (10.0 mL). The reaction mixture was stirred at room temperature for 3.5 hours before being filtered through a pad of celite, which was washed with EtOAc (2×30 mL). The filtrate was partitioned between EtOAc (40 mL), water (50 mL), and saturated aqueous sodium thiosulfate (50 mL). The aqueous layer was extracted with EtOAc (2×50 mL). The organic layers were combined, washed with brine (1×30 mL), dried over MgSO₄, filtered, and evaporated under reduced pressure. The resulting solid was purified by silica gel column (40 g) chromatography eluting with 0-40% EtOAc/hexanes to afford compound Int-14c. LCMS anal. calcd. for C₁₇H₁₇NO₆: 331.11; Found: 332.22 (M+1)⁺.

### Step D—Synthesis of Compound Int-14d

Compound Int-14c (106.7 mg, 0.322 mmol), sodium iodide (142.6 mg, 0.951 mmol), Int-13b (73.4 mg, 0.493 mmol), THF (1.5 mL) and water (1.5 mL) were combined and vigorously stirred at room temperature. Ten minutes later, indium (76.7 mg, 0.668 mmol) was added to the reaction mixture. After 17.5 hours, the reaction mixture was diluted with EtOAc (30 mL) and sonicated before being filtered through a pad of celite, which was washed with additional EtOAc (2×10 mL). The combined filtrate was dried over MgSO₄, filtered, and evaporated under reduced pressure. The residue was purified by silica gel column (12 g) chromatography eluting with 0-30% (25% EtOH/EtOAc)/hexanes to afford compound Int-14d. LCMS anal. calcd. for C₂₂H₂₇NO₆: 401.18; Found: 402.24 (M+1)⁺.

### Step E—Synthesis of Compound Int-14e

Compound Int-14d (63.3 mg, 0.158 mmol) and methylamine 2.0 M in THF (1.6 mL, 3.20 mmol) were combined and stirred at room temperature. After 2 days, methylamine 2.0 M in THF (1.6 mL, 3.20 mmol) was added to the reaction mixture. After an additional 3 days, the reaction mixture was evaporated under reduced pressure and the resulting residue was dissolved in methylamine 2.0 M in THF (1.6 mL, 3.20 mmol). The resulting solution was stirred at room temperature for 3 days before being heated to 40° C. for an additional 19.5 hours. The reaction mixture was cooled to room temperature and evaporated under reduced pressure. The resulting residue was dissolved in ACN/water, frozen, and lyophilized to afford compound Int-14e. LCMS anal. calcd. for C₂₂H₂₈N₂O₅: 400.20; Found: 401.31 (M+1)⁺.

### Step F—Synthesis of Compound Int-14f

p-Toluenesulfonic acid monohydrate (604.8 mg, 3.18 mmol) was added to a stirred solution of compound Int-14e (391.2 mg, 0.977 mmol) in MeOH (10.0 mL). The reaction mixture was stirred at room temperature for 24 hours before additional p-toluenesulfonic acid monohydrate (315.8 mg, 1.66 mmol) was added. After an additional 17 hours, additional p-toluenesulfonic acid monohydrate (213.8 mg, 1.12 mmol) was added to the reaction mixture. After 4 more days, the reaction mixture was concentrated to −4 mL under reduced pressure before being purified by reverse phase chromatography (50 g C18 RediSep™ gold column) eluting with 0-60% (ACN/water)+0.05% TFA. Clean product fractions were combined, frozen, and lyophilized. Product fractions that were significantly contaminated with tosic acid were combined and extracted with EtOAc (3×50 mL). Two spatulas of NaCl were added to the aqueous layer, which was extracted with EtOAc (50 mL) and DCM (50 mL). Three spatulas of NaCl were added to the aqueous layer, which was extracted with DCM (50 mL) and 10% MeOH/DCM (50 mL). Three more spatulas of NaCl were added to the aqueous layer, which was extracted with 10% MeOH/DCM (2×50 mL). The organic layers were combined, dried over MgSO₄, filtered, and evaporated under reduced pressure. The resulting residue was purified by reverse phase HPLC (Waters Sunfire C18 OBD, 10 μm, 30×150 mm column) eluting with 10-60% (ACN/water)+0.05% TFA. Product fractions were combined and evaporated under reduced pressure. The resulting residue was dissolved in MeOH/EtOAc, combined with the lyophilized product from the ISCO purification, and evaporated under reduced pressure to give compound Int-14f. LCMS anal. calcd. for C₁₄H₂N₂O₄: 280.14; Found: 281.23 (M+1)⁺.

### Step G—Synthesis of Compound Int-14g

tert-Butyldimethylsilyl chloride (475.0 mg, 3.15 mmol) was added to a stirred solution of compound Int-14f (371.6 mg, 1.326 mmol), imidazole (381.1 mg, 5.60 mmol), and DMAP (28.0 mg, 0.229 mmol) in DMF (6.6 mL). The reaction mixture was stirred at room temperature for 15 hours before being partitioned between EtOAc (150 mL) and water (40 mL). The organic layer was washed with water (2×40 mL) and brine (1×20 mL), dried over MgSO₄, filtered, and evaporated under reduced pressure. The resulting oil was purified by silica gel column (24 g) chromatography eluting with 0-40% (25% EtOH/EtOAc)/hexanes to afford compound Int-14g. LCMS anal. calcd. for C₂H₃₄N₂O₄Si: 394.23; Found: 395.42 (M+1)⁺.

### Step H—Synthesis of Compound Int-14h

NBS (277.1 mg, 1.557 mmol) was added to a stirred solution of compound Int-14g (306.9 mg, 0.778 mmol) in THF (7.8 mL). The reaction mixture was stirred at room temperature for 2 hours before additional NBS (69.3 mg, 0.389 mmol) was added. After an additional 1.5 hours, additional NBS (86.9 mg, 0.488 mmol) was added to the reaction mixture. After an additional hour, additional NBS (91.1 mg, 0.512 mmol) was added to the reaction mixture. 15 minutes later, the reaction mixture was partitioned between EtOAc (125 mL) and 0.1 M NaOH (50 mL). The organic layer was washed with 0.1 M NaOH (1×50 mL) and brine (1×20 mL). The organic layer diluted with DCM (˜75 mL). The combined aqueous layers were extracted with DCM (1×50 mL). The organic layers were combined, dried over MgSO₄, filtered, and evaporated under reduced pressure. The resulting residue was purified by column (24 g) chromatography eluting with 0-80% (25% EtOH/EtOAc)/hexanes followed by 10% MeOH/DCM to afford compound Int-14h. LCMS anal. calcd. for C₂₀H₃₂Br₂N₂O₄Si: 552.05; Found: 553.23 (M+1)⁺.

### Step I—Synthesis of Compound Int-14i

Cesium carbonate (1.1225 g, 3.45 mmol) was added to a stirred suspension of compound Int-14h (411.5 mg, 0.745 mmol) in DMSO (7.5 mL). The reaction mixture was stirred at room temperature for 17.5 hours before being partitioned between EtOAc (175 mL) and water (50 mL). The organic layer was washed with water (2×50 mL) and brine (1×20 mL). The combined aqueous layers were extracted with EtOAc (1×50 mL). The organic layers were combined, dried over MgSO₄, filtered, and evaporated under reduced pressure. The resulting solid was purified by silica gel column (24 g) chromatography eluting with 0-50% (25% EtOH/EtOAc)/hexanes to afford compound Int-14i. LCMS anal. calcd. for C₂₀H₃BrN₂O₄Si: 470.12, 472.12; Found: 471.27, 473.27 (M+1)⁺.

### Step J—Synthesis of Compound Int-14j

Compound Int-14i (300.8 mg, 0.638 mmol) and HCl 1.25 M in MeOH (6.5 mL, 8.13 mmol) were combined and heated to 40° C. with stirring. After 3 days, it was discovered that the cap had blown off and all of the solvent had evaporated. The resulting residue was purified by silica gel column (24 g) chromatography eluting with 0-10% MeOH/DCM to afford compound Int-14j. LCMS anal. calcd. for C₁₄H₁₇BrN₂O₄: 356.04, 358.04; Found: 357.16, 359.16 (M+1)⁺.

### Step K—Synthesis of Compound Int-14k

N,N-Diisopropylethylamine (0.2 mL, 1.145 mmol), 2,4-difluorobenzylamine (0.08 mL, 0.673 mmol), and 1,1′-bis(diphenylphosphino)ferrocene-palladium(II)dichloride dichloromethane complex (35.2 mg, 0.043 mmol) were added to a stirred solution of compound Int-14j (80.3 mg, 0.225 mmol) in DMSO (2.3 mL). The reaction mixture was degassed (3×) and placed under nitrogen before being degassed and placed under a carbon monoxide balloon. The reaction mixture was stirred at 100° C. for 16.5 hours. The reaction mixture was cooled to room temperature, diluted with MeOH, and filtered (0.45 μm syringe filter) before being purified by reverse phase chromatography (50 g C18 RediSep™ gold column) eluting with 0-100% (ACN/Water)+0.05% TFA. Product fractions were combined, frozen, and lyophilized to give an amber solid, which was further purified by chiral preparative SFC (ChiralPak AD-H, 21 250×mm column, 50 g/min, 120 bar, 35% (1:1 ACN/MeOH+0.2% DIPA)/CO₂, 40° C.) to afford isomer A of compound Int-14k (st eluting component), isomer B of compound Int-14k (2^{nd} eluting component), isomer C of compound Int-14k (3^{rd} eluting component), and isomer D of compound Int-14k (4^{th} eluting component). Isomer C and Isomer D were each separated a second time using the chiral preparative SFC conditions described above to afford sufficient purity. LCMS anal. calcd. for C₂₂H₂₃F₂N₃O₅: 447.16; Found: 448.30 (M+1)⁺.

### Step L—Synthesis of Compound 37, Compound 38, Compound 39, and Compound 40

Isomer A of compound Int-14k (37.5 mg, 0.084 mmol), magnesium bromide (160.2 mg, 0.870 mmol), and acetonitrile (1.8 mL) were combined and stirred at room temperature for 3 hours. The reaction mixture was diluted with MeOH and filtered (0.45 μm syringe filter) before being purified by reverse phase HPLC (Waters Sunfire C18 OBD, 10 μm, 30×150 mm column) eluting with 10-90% (ACN/water)+0.05% TFA. Product fractions were combined and concentrated under reduced pressure until most of the acetonitrile had been removed. The remaining aqueous solution was extracted with DCM (4×−5 mL). The organic layers were sequentially dried over sodium sulfate, filtered, combined, and evaporated under reduced pressure. The resulting residue was dissolved in ACN/water, frozen, and lyophilized to give compound 37. ¹H NMR (500 MHz, CD₃OD): δ 7.49-7.42 (m, 1H); 7.02-6.91 (m, 2H); 5.68 (t, J=7.9 Hz, 1H); 4.70-4.61 (m, 2H); 3.91 (d, J=13.3 Hz, 1H); 3.84 (d, J=13.3 Hz, 1H); 3.18 (s, 3H); 2.91 (dd, J=13.1, 7.7 Hz, 1H); 2.03 (dd, J=12.9, 8.1 Hz, 1H); 1.84 (dq, J=14.8, 7.4 Hz, 1H); 1.74 (dq, J=14.7, 7.5 Hz, 1H); 0.85 (t, J=7.5 Hz, 3H). LCMS anal. calcd. for C₂₁H₂F₂N₃O₅: 433.14; Found: 434.20 (M+1)⁺.

Following essentially the method employed to produce compound 37 in step L of example 14, compound 38 was prepared from Isomer B of compound Int-14k. ¹H NMR (500 MHz, CD₃OD): δ 7.50-7.42 (m, 1H); 7.01-6.91 (m, 2H); 5.72 (d, J=7.7 Hz, 1H); 4.69-4.60 (m, 2H); 3.87 (d, J=13.4 Hz, 1H); 3.75 (d, J=13.4 Hz, 1H); 3.18 (s, 3H); 2.42 (d, J=13.9 Hz, 1H); 2.29 (dd, J=13.9, 7.8 Hz, 1H); 1.99 (q, J=7.4 Hz, 2H); 1.03 (t, J=7.5 Hz, 3H). LCMS anal. calcd. for C₂₁H₂₁F₂N₃O₅: 433.14; Found: 434.22 (M+1)⁺.

Following essentially the method employed to produce compound 37 in step L of example 14, compound 39 was prepared from Isomer C of compound Int-14k. ¹H NMR (500 MHz, CD₃OD): δ 7.50-7.42 (m, 1H); 7.01-6.91 (m, 2H); 5.72 (d, J=7.8 Hz, 1H); 4.69-4.60 (m, 2H); 3.87 (d, J=13.4 Hz, 1H); 3.75 (d, J=13.4 Hz, 1H); 3.18 (s, 3H); 2.42 (d, J=13.9 Hz, 1H); 2.29 (dd, J=13.9, 7.8 Hz, 1H); 1.99 (q, J=7.4 Hz, 2H); 1.03 (t, J=7.5 Hz, 3H). LCMS anal. calcd. for C₂₁H₂₁F₂N₃O₅: 433.14; Found: 434.23 (M+1)⁺.

Following essentially the method employed to produce compound 40 in step L of example 14, compound 40 was prepared from Isomer D of compound Int-14k. ¹H NMR (500 MHz, CD₃OD): δ 7.50-7.42 (m, 1H); 7.02-6.91 (m, 2H); 5.68 (t, J=7.9 Hz, 1H); 4.70-4.60 (m, 2H); 3.92 (d, J=13.2 Hz, 1H); 3.84 (d, J=13.3 Hz, 1H); 3.18 (s, 3H); 2.91 (dd, J=13.1, 7.7 Hz, 1H); 2.04 (dd, J=12.9, 8.1 Hz, 1H); 1.84 (dq, J=14.8, 7.5 Hz, 1H); 1.74 (dq, J=14.7, 7.4 Hz, 1H); 0.85 (t, J=7.5 Hz, 3H). LCMS anal. calcd. for C₂₁H₂F₂N₃O₅: 433.14; Found: 434.27 (M+1)⁺.

### Example 15

### Preparation of Compounds 41-44

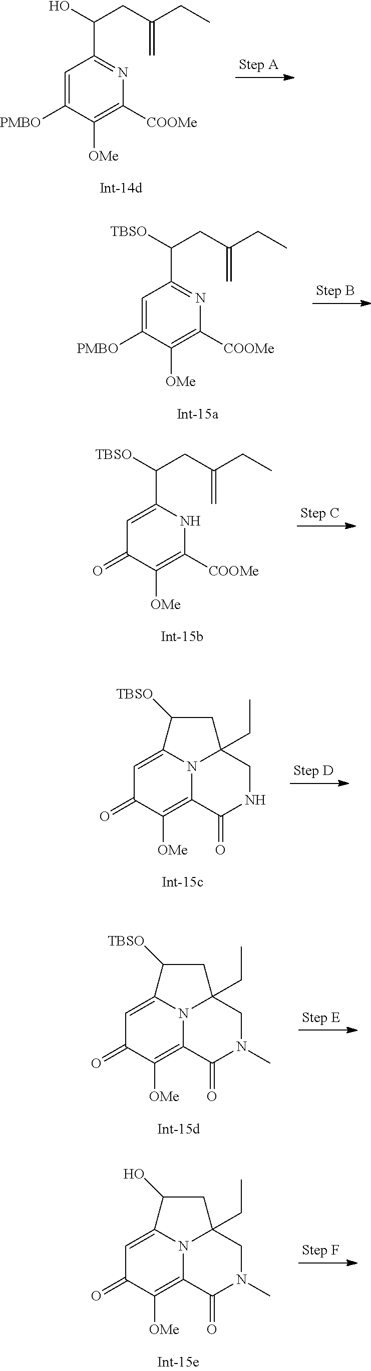

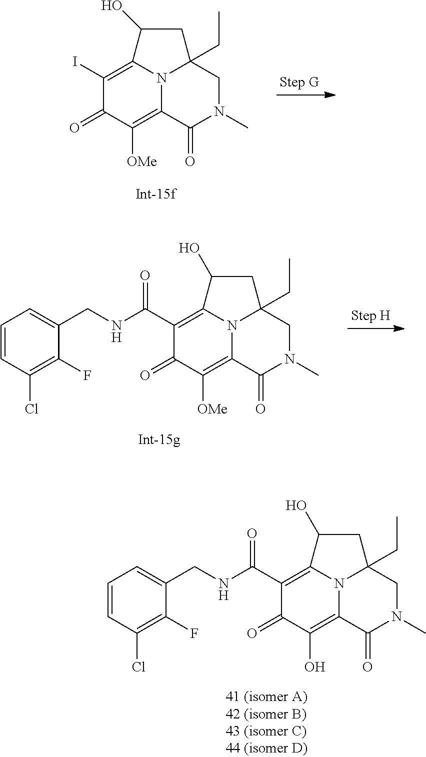

### Step A—Synthesis of Compound Int-15a

To a solution of compound Int-14d (15.7 g, 39.1 mmol) in DCM (200 mL) was added DMAP (2.389 g, 19.55 mmol), 2,6-dimethylpyridine (12.57 g, 117 mmol) and tert-butyldimethylsilyl trifluoromethanesulfonate (20.68 g, 78 mmol) at 0° C. The reaction mixture was stirred at 20° C. for 2 hours before being quenched with water (50 mL) and extracted with DCM (100 mL). The combined organic phase was dried over Na₂SO₄, filtered, concentrated in vacuo. The crude product was purified by flash silica gel chromatography eluting with 0-20% EtOAc/petroleum ether to afford compound Int-15a. LCMS anal. calcd. for C₂H₄NO₆Si: 515.3; Found: 516.9 (M+1)⁺.

### Step B Synthesis of Compound Int-15b

To a solution of compound Int-15a (8.4 g, 16.29 mmol) in DCM (85 mL) was added TFA (8.5 mL) at 0° C. The reaction mixture was stirred at 20° C. for 2 hours before being quenched with saturated aqueous NaHCO₃ (50 mL) and diluted with water (50 mL). The aqueous phase was extracted with DCM (100 mL) and combined organic layers were dried over Na₂SO₄, filtered, and concentrated in vacuo. The crude product was purified by flash silica gel chromatography (120 g column) eluting with 0-50% EtOAc/petroleum ether to afford compound Int-15b. LCMS anal. calcd. for C₂₀H₃₃NO₅Si: 395.2; Found: 396.5 (M+1)⁺.

### Step C—Synthesis of Compound Int-15c

To a mixture of compound Int-15b (5.3 g, 13.40 mmol) and O-(2,4-dinitrophenyl)hydroxylamine (8.00 g, 40.2 mmol) in CF₃CH₂OH (80 mL) was added bis[rhodium(α,α,α′,α′-tetramethyl-1,3-benzenedipropionic acid)] (0.204 g, 0.268 mmol). The reaction mixture was stirred at 65° C. under nitrogen for 6 hours before being concentrated in vacuo. The resulting residue was purified by flash silica gel chromatography (40 g column) eluting with 0-10% MeOH/DCM to afford compound Int-15c. LCMS anal. calcd. for C₁₉H₃₀N₂O₄Si: 378.2; Found: 379.0 (M+1)⁺.

### Step D—Synthesis of Compound Int-15d

To a mixture of compound Int-15d (1.35 g, 3.57 mmol) in DMF (25 mL) was added NaH (0.428 g, 10.70 mmol) and iodomethane (0.666 mL, 10.70 mmol) at 0° C. The reaction mixture was stirred at 20° C. for 2 hours before being quenched with 1 N HCl (0.5 mL) and concentrated in vacuo. The crude product was purified by preparative TLC plate eluting with 10% MeOH/DCM to afford compound Int-15d. LCMS anal. calcd. for C₂H₃₂N₂O₄Si: 392.2; Found: 393.3 (M+1)⁺.

### Step E—Synthesis of Compound Int-15e

To a mixture of compound Int-15d (2.5 g, 6.37 mmol) in THF (40 mL) was added TBAF (12.74 mL, 12.74 mmol) at 0° C. The reaction mixture was stirred at 20° C. for 2 hours before being concentrated in vacuo. The crude product was purified by preparative TLC plate eluting with 12% MeOH/DCM to afford compound Int-15e. LCMS anal. calcd. for C₁₄H₁₈N₂O₄: 278.1; Found: 279.1 (M+1)⁺.

### Step F—Synthesis of Compound Int-15f

To a solution of compound Int-15e (1.7 g, 6.11 mmol) in MeOH (30 mL) was added m-CPBA (5.27 g, 24.43 mmol) and NIS (5.50 g, 24.43 mmol). The reaction mixture was stirred at 80° C. for 1.5 hours before being quenched with saturated aqueous Na₂SO₃ (15 mL). The reaction mixture was filtered and purified by preparative reverse phase HPLC (Phenomenex Synergi Max-RP, 10 μm, 50×250 mm column) eluting with 0-20% ACN/(water+0.1% TFA) to afford compound Int-15f. LCMS anal. calcd. for C₁₄H₁₇IN₂O₄: 404.0; Found: 404.8 (M+1)⁺.

### Step G—Synthesis of Compound Int-15g

To a solution of compound Int-15f (300 mg, 0.742 mmol) in DMSO (5 mL) was added (3-chloro-2-fluorophenyl)methanamine (237 mg, 1.484 mmol), Pd(Ph₃P)₄ (429 mg, 0.371 mmol), and DIEA (0.648 mL, 3.71 mmol). The reaction mixture was degassed and purged with CO (3×) before being stirred at 80° C. for 1.5 hours under a CO balloon. The reaction mixture was filtered and purified by preparative reverse phase HPLC (Boston Green ODS, 5 μm, 30×150 mm column) eluting with 40-60% ACN/(water+0.1% TFA). This material was further purified by chiral preparative SFC (DAICEL CHIRALPAK AS-H, 5 μm, 30×250 mm column, 60 mL/min, 40% (EtOH+0.1% NH₃H₂O)/CO₂, 220 nm) to afford a mixture of Isomer A and Isomer B of compound Int-15g (st eluting component), Isomer C of compound Int-15g (2^{nd} eluting component), and Isomer D of compound Int-15g (3^{rd} eluting component). The mixture of Isomer A and Isomer B of compound Int-15g was further purified by preparative chiral SFC (DAICEL CHIRALPAK IC, 10 μm, 30×250 mm column, 50 mL/min, 50% (EtOH+0.1% NH₃H₂O)/CO₂, 220 nm) to afford Isomer A of compound Int-15g (1st eluting component) and Isomer B of compound Int-15g (2^{nd} eluting component). Isomer A of compound Int-15g was further purified by chiral preparative SFC (DAICEL CHIRALPAK AD, 10 μm, 30×250 mm, 70 mL/min, 50% (MeOH+0.1% NH₃H₂O)/CO₂, 220 nm) to afford Isomer A of Int-15g. LCMS anal. calcd. for C₂₂H₂₃C₁FN₃O₅: 463.1; Found: 464.1 (M+1)⁺.

### Step H—Synthesis of Compound 41, Compound 42, Compound 43, and Compound 44

To a solution of Isomer B of compound Int-15g (50 mg, 0.108 mmol) in acetonitrile (3 mL) was added magnesium bromide (99 mg, 0.539 mmol). The mixture was stirred at 10° C. for 12 hours before being diluted with MeOH (0.5 mL) and purified by preparative reverse phase HPLC (Boston Green ODS, 5 μm, 30×150 mm column) eluting with 33-63% ACN/(water+0.1% TFA). Product fractions were co-evaporated with toluene two times to afford compound 42. ¹H NMR (400 MHz, CDCl₃) δ:10.99 (br s, 1H); 7.30 (q, J=6.6 Hz, 2H); 7.11-6.98 (m, 1H); 5.67 (d, J=7.9 Hz, 1H); 4.86-4.63 (m, 2H); 3.69 (s, 2H); 3.20 (s, 3H); 2.46 (d, J=14.0 Hz, 1H); 2.20-2.14 (m, 1H); 2.04-1.88 (m, 2H); 1.09-1.00 (m, 3H). LCMS anal. calcd. for C₂₁H₂₁ClFN₃O₅: 449.1; Found: 450.2 (M+1)⁺.

Following essentially the method employed to produce compound 42 in step H of example 15, compound 41 was prepared from Isomer A of compound Int-15g. ¹H NMR (400 MHz, CDCl₃) δ: 11.43 (br s, 1H); 7.33-7.26 (m, 2H); 7.04 (t, J=7.9 Hz, 1H); 5.66 (t, J=7.9 Hz, 1H); 4.72 (br d, J=5.7 Hz, 2H); 3.87 (d, J=13.2 Hz, 1H); 3.63 (d, J=12.7 Hz, 1H); 3.20 (s, 3H); 2.82 (dd, J=13.2, 7.9 Hz, 1H); 2.35 (s, 1H); 2.07 (dd, J=13.2, 7.9 Hz, 1H); 1.86-1.67 (m, 2H); 0.85 (t, J=7.5 Hz, 3H). LCMS anal. calcd. for C₂₁H₂₁ClFN₃O₅: 449.1; Found: 450.2 (M+1)⁺.

Following essentially the method employed to produce compound 42 in step H of example 15, compound 43 was prepared from Isomer C of compound Int-15g. ¹H NMR (400 MHz, CDCl₃) δ:11.42 (br s, 1H); 7.33-7.27 (m, 2H); 7.08-7.00 (m, 1H); 5.66 (t, J=7.9 Hz, 1H); 4.72 (br d, J=5.7 Hz, 2H); 3.87 (d, J=13.2 Hz, 1H); 3.63 (d, J=13.2 Hz, 1H); 3.20 (s, 3H); 2.82 (dd, J=13.2, 7.9 Hz, 1H); 2.07 (dd, J=13.2, 8.3 Hz, 1H); 1.86-1.68 (m, 2H); 0.85 (t, J 7.5 Hz, 3H). LCMS anal. calcd. for C₂₁H₂₁ClFN₃O₅: 449.1; Found: 450.2 (M+1)⁺.

Following essentially the method employed to produce compound 42 in step H of example 15, compound 44 was prepared from Isomer D of compound Int-15g. ¹H NMR (400 MHz, CDCl₃) δ: 10.99 (br s, 1H); 7.34-7.27 (m, 2H); 7.04 (t, J=7.9 Hz, 1H); 5.65 (d, J=7.9 Hz, 1H); 4.78-4.58 (m, 2H); 3.76-3.64 (m, 2H); 3.20 (s, 3H); 2.45 (d, J=14.0 Hz, 1H); 2.18 (dd, J 14.0, 8.3 Hz, 1H); 2.06-1.90 (m, 2H), 1.27 (br d, J=7.0 Hz, 1H), 1.01 (t, J=7.5 Hz, 3H). LCMS anal. calcd. for C₂₁H₂₁ClFN₃O₅: 449.1; Found: 450.2 (M+1)⁺.

### Example 16

### Preparation of Compounds 45-48

Starting from Int-15f, using essentially the same method described in Step G and Step H in example 15 with the exception of substituting with 2,4,6-trifluorobenzylamine, purifying by chiral preparative SFC (DAICEL CHIRALPAK AS-H, 5 μm, 30×250 mm column, 65 mL/min, 35% (EtOH+0.1% NH₃H₂O)/CO₂, 220 nm) to afford a mixture of Isomer A and Isomer B, Isomer C, and Isomer D, further purifying the mixture of Isomer A and Isomer B by chiral preparative SFC (DAICEL CHIRALPAK AD, 10 μm, 30×250 mm column, 70 mL/min, 40% (EtOH+0.1% NH₃H₂O)/CO₂, 220 nm) to afford Isomer A and Isomer B, and further purifying Isomer A by chiral preparative SFC (DAICEL CHIRALPAK AD, 10 μm, 30×250 mm column, 50 mL/min, 50% (MeOH+0.1% NH₃H₂O)/CO₂, 220 nm) in Step G, the following compounds were prepared:

| | | MS | |
|---|---|---|---|
| Compound # | Structure | (M + H) | ¹H NMR |
| 45 (Isomer A) | 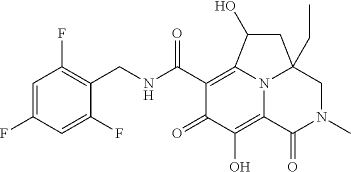 [SMILES: CCC12CC(O)c3c(C(=O)NCc4c(F)cc(F)cc4F)c(=O)c(O)c(n31)C(=O)N(C)C2] | 452.2 | (400 MHz, CDCl₃) δ: 11.32 (br s, 1H); 6.88 (br s, 1H); 6.67 (t, J = 8.1 Hz, 2H); 5.67 (t, J = 7.9 Hz, 1H); 4.85-4.46 (m, 2H); 3.85 (d, J = 13.2 Hz, 1H); 3.62 (d, J = 13.2 Hz, 1H); 3.19 (s, 3H); 2.81 (dd, J = 12.9, 7.7 Hz, 1H); 2.06 (dd, J = 12.9, 8.1 Hz, 1H); 1.84-1.65 (m, 2H); 0.83 (t, J = 7.5 Hz, 3H). |
| 46 (Isomer B) | 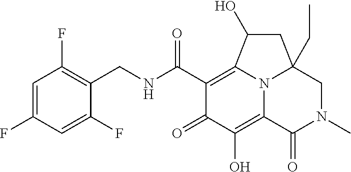 [SMILES: CCC12CC(O)c3c(C(=O)NCc4c(F)cc(F)cc4F)c(=O)c(O)c(n31)C(=O)N(C)C2] | 452.2 | (400 MHz, CDCl₃) δ: 10.88 (br s, 1H); 6.67 (t, J = 8.1 Hz, 2H); 5.66 (d, J = 7.9 Hz, 1H); 4.75- 4.53 (m, 2H); 3.84-3.57 (m, 2H); 3.19 (s, 3H); 2.44 (d, J = 13.6 Hz, 1H); 2.20-1.80 (m, 3H); 1.00 (t, J = 7.5 Hz, 3H). |
| 47 (Isomer C) | 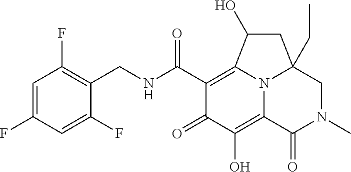 [SMILES: CCC12CC(O)c3c(C(=O)NCc4c(F)cc(F)cc4F)c(=O)c(O)c(n31)C(=O)N(C)C2] | 452.2 | (400 MHz, CDCl₃) δ: 11.32 (br s, 1H); 6.88 (br s, 1H); 6.67 (t, J = 8.1 Hz, 2H); 5.67 (t, J = 7.9 Hz, 1H); 4.61-4.75 (m, 2H); 3.85 (d, J = 13.2 Hz, 1H); 3.61 (d, J = 12.7 Hz, 1H); 3.19 (s, 3H); 2.80 (dd, J = 13.2, 7.9 Hz, 1H); 2.06 (dd, J = 13.2, 8.3 Hz, 1H); 1.87-1.63 (m, 2H); 0.83 (t, J = 7.5 Hz, 3H). |
| 48 (Isomer D) | 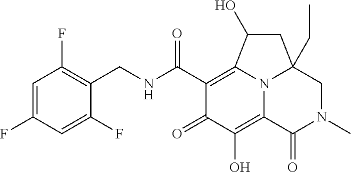 [SMILES: CCC12CC(O)c3c(C(=O)NCc4c(F)cc(F)cc4F)c(=O)c(O)c(n31)C(=O)N(C)C2] | 452.2 | (400 MHz, CDCl₃) δ: 10.90 (br s, 1H); 6.67 (t, J = 8.1 Hz, 2H); 5.68 (d, J = 7.9 Hz, 1H); 4.65 (br d, J = 5.3 Hz, 2H); 3.68 (s, 2H); 3.19 (s, 3H); 2.47 (s, 1H); 2.22- 1.88 (m, 3H), 1.00 (t, J = 7.5 Hz, 3H). |

### Example 17

### Preparation of Compounds 49-52

Starting from Int-15f, using essentially the same method described in Step G and Step H in example 15 with the exception of substituting with 2,3,6-trifluorobenzylamine, purifying by chiral preparative SFC (DAICEL CHIRALPAK AD, 10 m, 30×250 mm column, 50 mL/min, 35% (EtOH+0.10% NH₃H₂O)/CO₂, 220 nm) to afford Isomer A, Isomer B, and a mixture of Isomer C and Isomer D, and further purifying the mixture of Isomer C and D by chiral preparative SFC (DAICEL CHIRALPAK AD, 10 m, 30×250 mm column, 50 mL/min 25% (EtOH+0.100 NH₃H₂O)/CO₂, 220 nm) to afford isomer C and isomer Din Step G, the following compounds were prepared:

| | | MS | |
|---|---|---|---|
| Compound # | Structure | (M + H) | ¹H NMR |
| 49 (Isomer A) | 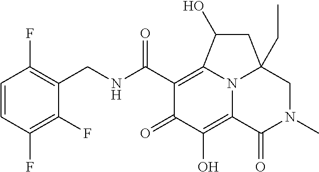 [SMILES: CCC12CC(O)c3c(C(=O)NCc4c(F)ccc(F)c4F)c(=O)c(O)c(n31)C(=O)N(C)C2] | 452.1 | (400 MHz, CDCl₃) δ: 11.38 (br s, 1H); 7.14-7.02 (m, 1H); 6.85 (br d, J = 7.5 Hz, 2H); 5.67 (t, J = 7.9 Hz, 1H); 4.93-4.66 (m, 2H); 3.85 (d, J = 13.2 Hz, 1H); 3.61 (d, J = 13.2 Hz, 1H); 3.19 (s, 3H); 2.80 (dd, J = 13.2, 7.9 Hz, 1H); 2.06 (dd, J = 12.9, 7.7 Hz, 1H); 1.85-1.62 (m, 2H); 0.83 (t, J = 7.5 Hz, 3H). |
| 50 (Isomer B) | 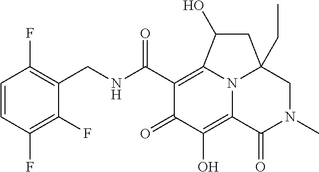 [SMILES: CCC12CC(O)c3c(C(=O)NCc4c(F)ccc(F)c4F)c(=O)c(O)c(n31)C(=O)N(C)C2] | 452.1 | (400 MHz, CDCl₃) δ: 11.38 (br s, 1H); 7.09-7.06 (m, 1H); 6.84 (br d, J = 7.5 Hz, 2H); 5.67 (t, J = 8.0 Hz, 1H); 4.78-4.71 (m, 2H); 3.85 (d, J = 13.2 Hz, 1H); 3.82 (d, J = 13.2 Hz, 1H); 3.19 (s, 3H); 2.80 (dd, J = 13.2, 8.0 Hz, 1H); 2.06 (dd, J = 13.2, 7.6 Hz, 1H); 1.79-1.70 (m, 2H); 0.83 (t, J = 7.6 Hz, 3H). |
| 51 (Isomer C) | 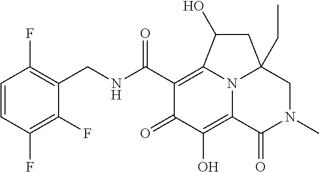 [SMILES: CCC12CC(O)c3c(C(=O)NCc4c(F)ccc(F)c4F)c(=O)c(O)c(n31)C(=O)N(C)C2] | 452.1 | (400 MHz, CDCl₃) δ: 10.93 (br s, 1H); 7.11-6.97 (m, 1H); 6.84 (br t, J = 8.3 Hz, 1H); 5.65 (d, J = 8.3 Hz, 1H); 5.62-5.49 (m, 1H); 4.72 (br d, J = 4.4 Hz, 2H); 3.84-3.58 (m, 2H); 3.19 (s, 3H); 2.44 (d, J = 14.0 Hz, 1H); 2.27- 1.82 (m, 3H); 1.00 (t, J = 7.5 Hz, 3H). |
| 52 (Isomer D) | 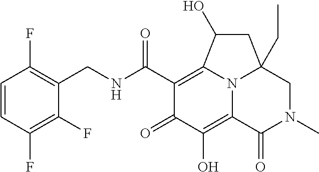 [SMILES: CCC12CC(O)c3c(C(=O)NCc4c(F)ccc(F)c4F)c(=O)c(O)c(n31)C(=O)N(C)C2] | 452.1 | (400 MHz, CDCl₃) δ: 10.92 (br s, 1H); 7.12-6.99 (m, 1H); 6.84 (br t, J = 9.4 Hz, 1H); 5.64 (d, J = 7.9 Hz, 1H); 4.75-4.58 (m, 2H); 3.87-3.57 (m, 2H); 3.19 (s, 3H); 2.44 (d, J = 13.6 Hz, 1H); 2.19 (dd, J = 14.0, 7.9 Hz, 1H); 2.05-1.83 (m, 2H); 1.00 (t, J = 7.5 Hz, 3H). |

### Example 18

### Preparation of Compounds 53-56

Starting from Int-15f, using essentially the same method described in Step G and Step H in example 15 with the exception of substituting with (3-chloro-2,6-difluorophenyl)methanamine, purifying by chiral preparative SFC (DAICEL CHIRALPAK AD, 10 μm, 30×250 mm column, 70 mL/min, 40% (EtOH+0.1% NH₃H₂O)/CO₂, 220 nm) to afford Isomer A, a mixture of Isomer B and Isomer C, and Isomer D, further purifying the mixture of Isomer B and C by chiral preparative SFC (DAICEL CHIRALPAK OJ-H, 5 μm, 30×250 mm column, 50 mL/min, 30% (EtOH+0.1% NH₃H₂O)/CO₂, 220 nm) to afford isomer B and isomer C, and further purifying Isomer B by chiral preparative SFC (DAICEL CHIRALPAK OJ-H, 5 μm, 30×250 mm column, 50 mL/min 30% (EtOH+0.1% NH₃H₂O)/CO₂, 220 nm) in step G, the following compounds were prepared:

| | | MS | |
|---|---|---|---|
| Compound # | Structure | (M + H) | ¹H NMR |
| 53 (Isomer A) | 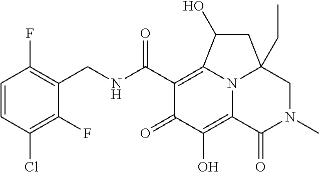 [SMILES: CCC12CC(O)c3c(C(=O)NCc4c(F)ccc(Cl)c4F)c(=O)c(O)c(n31)C(=O)N(C)C2] | 468.1 | (400 MHz, CDCl₃) δ: 11.38 (br s, 1H); 7.33-7.27 (m, 1H); 6.90-6.78 (m, 2H); 5.67 (t, J = 7.9 Hz, 1H); 4.81- 4.67 (m, 2H); 3.85 (d, J = 13.6 Hz, 1H); 3.61 (d, J = 13.2 Hz, 1H); 3.19 (s, 3H); 2.82-2.62 (m, 1H); 2.06 (dd, J = 13.2, 8.3 Hz, 1H); 1.85-1.73 (m, 2H); 0.83 (t, J = 7.5 Hz, 3H). |
| 54 (Isomer B) | 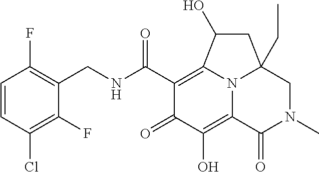 [SMILES: CCC12CC(O)c3c(C(=O)NCc4c(F)ccc(Cl)c4F)c(=O)c(O)c(n31)C(=O)N(C)C2] | 468.1 | (400 MHz, CDCl₃) δ: 11.38 (br s, 1H); 7.30 (td, J = 8.6, 5.7 Hz, 1H); 7.00-6.77 (m, 2H); 5.66 (t, J = 7.7 Hz, 1H); 4.81-4.65 (m, 2H); 3.86 (d, J = 13.2 Hz, 1H); 3.61 (d, J = 12.7 Hz, 1H); 3.19 (s, 3H); 2.80 (dd, J = 13.2, 7.5 Hz, 1H); 2.06 (dd, J = 13.2, 7.9 Hz, 1H); 1.81-1.68 (m, 3H); 0.83 (t, J = 7.5 Hz, 3H). |
| 55 (Isomer C) | 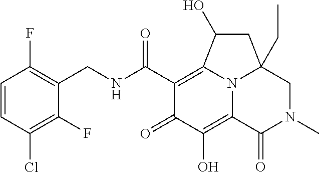 [SMILES: CCC12CC(O)c3c(C(=O)NCc4c(F)ccc(Cl)c4F)c(=O)c(O)c(n31)C(=O)N(C)C2] | 468.1 | (400 MHz, CDCl₃) δ: 10.91-10.86 (m, 1H); 7.31 (td, J = 8.6, 5.7 Hz, 1H); 6.88 (t, J = 8.8 Hz, 1H); 5.60 (d, J = 7.9 Hz, 1H); 4.79-4.62 (m, 2H); 3.85- 3.60 (m, 2H); 3.18 (s, 3H); 2.42 (d, J = 14.0 Hz, 1H); 2.24-2.15 (m, 1H); 2.08-1.87 (m, 2H); 0.99 (t, J = 7.5 Hz, 3H). |
| 56 (Isomer D) | 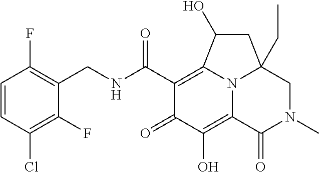 [SMILES: CCC12CC(O)c3c(C(=O)NCc4c(F)ccc(Cl)c4F)c(=O)c(O)c(n31)C(=O)N(C)C2] | 468.1 | (400 MHz, CDCl₃) δ: 11.09-10.75 (m, 1H); 7.31 (td, J = 8.6, 5.7 Hz, 1H); 6.96-6.79 (m, 1H); 5.61 (d, J = 7.9 Hz, 1H); 4.83-4.59 (m, 2H); 3.84-3.58 (m, 2H); 3.18 (s, 3H); 2.42 (d, J = 13.6 Hz, 1H); 2.21 (br dd, J = 13.8, 8.1 Hz, 1H); 1.99-1.81 (m, 2H); 0.99 (t, J = 7.5 Hz, 3H). |

### Example 19

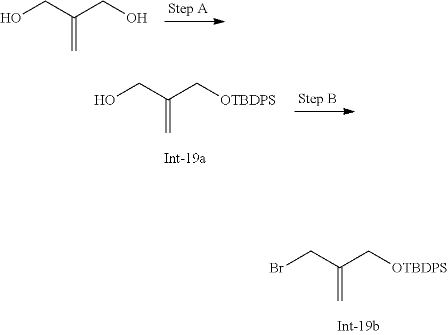

### Step A—Synthesis of Compound Int-19a

TBDPSCl (9.0 mL, 35.0 mmol) was added dropwise to a stirred hazy solution of 2-methylenepropane-1,3-diol (3 g, 34.1 mmol) and imidazole (4.70 g, 69.0 mmol) in DCM (340 mL). The reaction mixture was stirred at room temperature overnight. The following morning, the reaction mixture was concentrated under reduced pressure (˜80 mL) before being filtered through a pad of celite, rinsing over with additional DCM/MeOH. The filtrate was evaporated under reduced pressure. The resulting oil was purified by silica gel column (220 g) chromatography eluting with 0-20% EtOAc/hexanes to afford compound Int-19a. ¹H NMR (500 MHz, CDCl₃): δ 7.69 (d, J=7.4 Hz, 4H); 7.49-7.37 (m, 6H); 5.16 (app. s, 1H); 5.13 (app. s, 1H); 4.27 (s, 2H); 4.19 (d, J=6.2 Hz, 2H); 1.80 (t, J=6.1 Hz, 1H); 1.08 (s, 9H).

### Step B—Synthesis of Compound Int-19b

Triphenylphosphine (3.87 g, 14.75 mmol) and carbon tetrabromide (5.12 g, 15.44 mmol) were added to a stirred solution of Int-19a (3.8542 g, 11.80 mmol) in DCM (118.0 mL). The reaction mixture was stirred at room temperature for 1.5 hours before being concentrated under reduced pressure to 15-20 mL. The concentrated reaction mixture was purified by silica gel column (120 g) chromatography eluting with 0-10% DCM/hexanes to afford compound Int-19b. ¹H NMR (500 MHz, CDCl₃): δ 7.69 (d, J=7.0 Hz, 4H); 7.49-7.37 (m, 6H); 5.32 (app. s, 1H); 5.30 (app. s, 1H); 4.31 (s, 2H); 4.04 (s, 2H); 1.08 (s, 9H).

### Example 20

### Preparation of Compounds 57-60

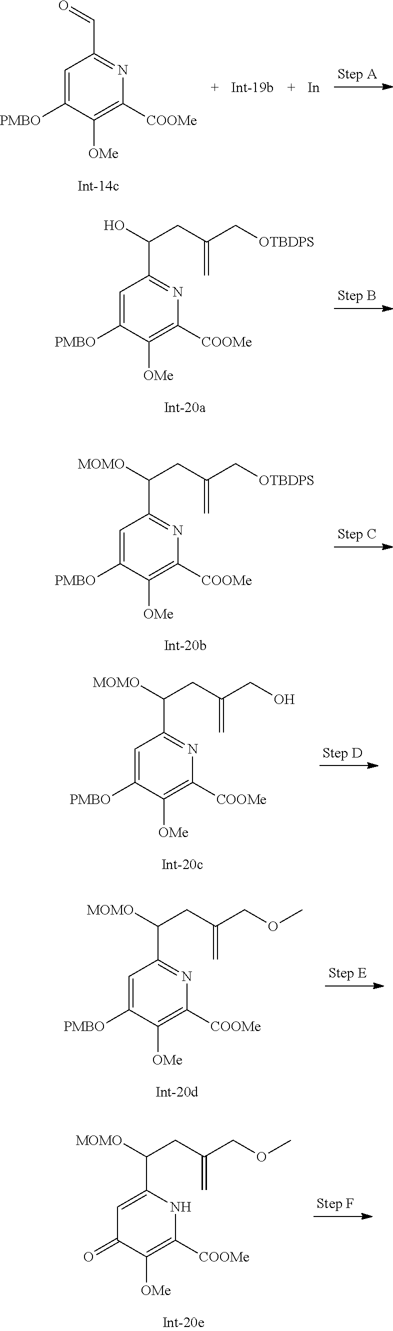

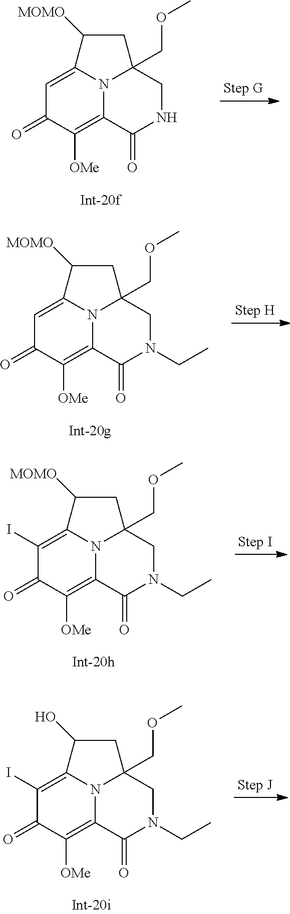

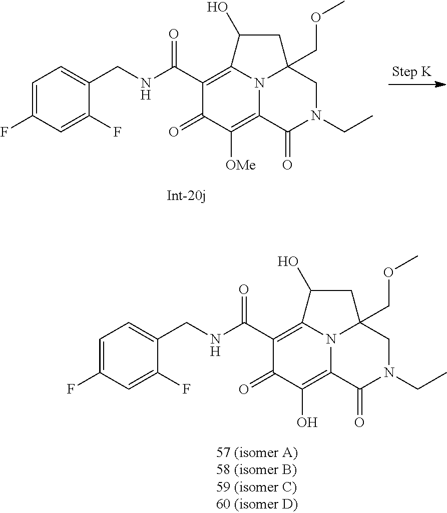

### Step A Synthesis of Compound Int-20a

Compound Int-14c (503.2 mg, 1.519 mmol), sodium iodide (762.5 mg, 5.09 mmol), compound Int-19b (1.1612 g, 2.98 mmol), THF (6.0 mL), and water (6.0 mL) were combined. The reaction mixture was vigorously stirred at room temperature for 10 minutes prior to the addition of indium (392.5 mg, 3.42 mmol). After 16.5 hours, the reaction mixture was diluted with EtOAc (75 mL) before being filtered through a pad of celite, rinsing with additional EtOAc (2×50 mL). The combined filtrate was poured into a 250 mL separation funnel and the layers were allowed to separate. The organic layer was dried over MgSO₄, filtered, and evaporated under reduced pressure. The resulting foam was purified by silica gel column (40 g) chromatography eluting with 0-80% (25% EtOH/EtOAc)/hexanes to afford compound Int-20a. LCMS anal. calcd. for C₃₇H₄₃NO₇Si: 641.28; Found: 642.42 (M+1)⁺.

### Step B Synthesis of Compound Int-20b

A stirred solution of compound Int-20a (886.0 mg, 1.380 mmol) in DCM (14.0 mL) was cooled to 0° C. in an ice bath. N,N-diisopropylethylamine (1.2 mL, 6.87 mmol), MOMCl (0.52 mL, 6.85 mmol) (added dropwise), and DMAP (37.2 mg, 0.304 mmol) were added to the reaction mixture. After 50 minutes, the reaction mixture was removed from the bath and allowed to warm to room temperature. 1.5 days later, the reaction mixture was concentrated under reduced pressure (˜6 mL remaining) before being purified by silica gel column (40 g) chromatography eluting with 0-20% (25% EtOH/EtOAc)/hexanes to afford compound Int-20b. LCMS anal. calcd. for C₃₉H₄₇NO₈Si: 685.31; Found: 686.51 (M+1)⁺.

### Step C—Synthesis of Compound Int-20c

TBAF 1.0 M in THF (1.7 mL, 1.700 mmol) was added to a stirred solution of compound Int-20b (866.6 mg, 1.263 mmol) in THF (11.0 mL). The reaction mixture was stirred at room temperature. After 4.5 hours, the reaction mixture was evaporated under reduced pressure. The resulting product was purified by silica gel column (40 g) chromatography eluting with 0-50% (25% EtOH/EtOAc)/hexanes to afford compound Int-20c. LCMS anal. calcd. for C₂₃H₂₉NO₈: 447.19; Found: 448.23 (M+1)⁺.

### Step D—Synthesis of Compound Int-20d

Iodomethane (0.21 mL, 3.36 mmol) and sodium hydride 60% dispersion in oil (107.1 mg, 2.68 mmol) were sequentially added to a stirred solution of compound Int-20c (498.1 mg, 1.113 mmol) in THF (11.0 mL) that had been cooled to 0° C. in an ice bath. After 50 minutes, the reaction mixture was removed from the ice bath and allowed to warm to room temperature. Following an additional 20 minutes, the reaction mixture was cooled to 0° C. in an ice bath before being diluted with EtOAc (50 mL). 1.0 M HCl (3 mL, 3 mmol) was diluted to 50 mL with additional water. About 10-15 mL of this HCl solution was slowly added to the reaction. The reaction mixture was removed from the ice bath and immediately partitioned between EtOAc (50 mL) and the remaining diluted HCl solution. The aqueous layer was extracted with EtOAc (2×50 mL). The organic layers were combined, dried over MgSO₄, filtered, and evaporated under reduced pressure. The resulting oil was dissolved in MeOH (10.0 mL) and TMS-Diazomethane 2.0 M in diethyl ether (2.0 mL, 4.00 mmol) was added dropwise. The reaction mixture was stirred at room temperature for 2 hours before being evaporated under reduced pressure. The resulting product was purified by silica gel column (40 g) chromatography eluting with 0-30% (25% EtOH/EtOAc)/hexanes to afford compound Int-20d. LCMS anal. calcd. for C₂₄H₃₁NO₈: 461.20; Found: 462.33 (M+1)⁺.

### Step E—Synthesis of Compound Int-20e

4-methylbenzenesulfonic acid hydrate (281.3 mg, 1.479 mmol) was added to a stirred solution of compound Int-20d (502.8 mg, 1.089 mmol) in MeOH (11.0 mL). The reaction mixture was stirred at room temperature for 20 hours. Sodium bicarbonate (123.9 mg, 1.475 mmol) and triethylamine (0.21 mL, 1.507 mmol) were added to the reaction mixture, which was placed in the freezer over the weekend. The reaction mixture was removed from the freezer, allowed to warm to room temperature, filtered (0.45 μm syringe filter), and diluted with MeOH before being purified by reverse phase HPLC (Waters Sunfire C18 OBD, 10 μm, 30×150 mm column) eluting with 0-60% (ACN/water)+0.05% TFA. Product fractions were combined and concentrated under reduced pressure until water started to evaporate. The remaining aqueous solution (˜100 mL) was extracted with DCM (4×50 mL). The organic layers were combined, dried over Na₂SO₄, filtered, and evaporated under reduced pressure to afford compound Int-20e. LCMS anal. calcd. for C₁₆H₂₃NO₇: 341.15; Found: 342.32 (M+1)⁺.

### Step F—Synthesis of Compound Int-20f

Bis[rhodium(α,α,α′,α′-tetramethyl-1,3-benzenedipropionic acid)] (21.6 mg, 0.028 mmol) and O-(2,4-dintrophenyl)hydroxylamine (290.7 mg, 1.460 mmol) were added to a stirred solution of compound Int-20e (318.9 mg, 0.934 mmol) in 2,2,2-trifluoroethanol (9.5 mL). The reaction mixture was stirred at room temperature for 3 hours. Additional bis[rhodium(α,α,α′,α′-tetramethyl-1,3-benzenedipropionic acid)] (25.8 mg, 0.034 mmol) and O-(2,4-dinitrophenyl)hydroxylamine (286.5 mg, 1.44 mmol) were added to the reaction mixture. After an additional 21 hours, the reaction mixture was evaporated under reduced pressure. The resulting residue was purified by silica gel column (40 g) chromatography eluting with 0-100% (90:9:1 DCM/MeOH/NH₄₀H)/DCM to afford compound Int-20f. LCMS anal. calcd. for C₁₅H₂₀N₂O₆: 324.13; Found: 325.21 (M+1)⁺.

### Step G—Synthesis of Compound Int-20g

Cesium carbonate (615.0 mg, 1.888 mmol) and iodoethane (65 μl, 0.804 mmol) were added to a stirred solution of compound Int-20f (199.5 mg, 0.615 mmol) in DMSO (6.0 mL). The reaction mixture was stirred at room temperature for 3.5 hours before being filtered (0.45 μm syringe filter) and purified by reverse phase HPLC (Waters Sunfire C18 OBD, 10 μm, 30×150 mm column) eluting with 0-70% (ACN/water)+0.05% TFA. The product fractions were combined, partially concentrated under reduced pressure, frozen, and lyophilized to afford compound Int-20g. LCMS anal. calcd. for C₁₇H₂₄N₂O₆: 352.16; Found: 353.28 (M+1)⁺.

### Step H—Synthesis of Compound Int-20h

NIS (237.5 mg, 1.056 mmol) and m-CPBA (217.1 mg, 0.969 mmol) were added to a stirred solution of compound Int-20g (199.7 mg, 0.567 mmol) in MeOH (5.6 mL). The reaction mixture was heated to 70° C. for 2 hours before being cooled to room temperature and evaporated under reduced pressure. The resulting solid was purified by silica gel column (24 g) chromatography eluting with 04% MeOH/DCM to afford compound Int-20h. LCMS anal. calcd. for C₁₇H₂₃IN₂O₆: 478.06; Found: 479.09 (M+1)⁺.

### Step I—Synthesis of Compound Int-20i

Compound Int-20h (271 mg, 0.567 mmol) was dissolved in HCl 1.25 M in MeOH (6.0 mL, 7.50 mmol) and heated to 40° C. After 18.5 hours, the reaction mixture was cooled to room temperature and evaporated under reduced pressure. The resulting solid was purified by silica gel column (24 g) chromatography eluting with 0-10% MeOH/DCM to afford compound Int-20i. LCMS anal. calcd. for C₁₅H₁₉IN₂O₅: 434.03; Found: 435.05 (M+1)⁺.

### Step J-Synthesis of Compound Int-20j

Bis(2-diphenylphosphinophenyl)ether (8.9 mg, 0.017 mmol), N,N-diisopropylethylamine (62 μl, 0.355 mmol), 2,4-difluorobenzylamine (25 μl, 0.210 mmol), and Pd(OAc)₂ (9.5 mg, 0.042 mmol) were added to a stirred solution of compound Int-20i (30.1 mg, 0.069 mmol) in DMSO (1.0 mL). The reaction mixture was degassed (3×) and placed under nitrogen before being degassed and placed under a carbon monoxide balloon. The reaction mixture was stirred at 100° C. for 16 hours before being cooled to room temperature, filtered (0.45 μm syringe filter), diluted with MeOH, and purified by reverse phase HPLC (Waters Sunfire C18 OBD, 10 μm, 30×150 mm column) eluting with 10-90% (ACN/water)+0.05% TFA. Product fractions were combined, frozen, and lyophilized, which was further purified by chiral preparative SFC (ChiralPak AD-H, 21×250 mm column, 70 g/min, 120 bar, 25% IPA/CO₂, 40° C.) to afford Isomer A of compound Int-20j (st eluting component), Isomer B of compound Int-20j (2^{nd} eluting component), Isomer C of compound Int-20j (3^{rd} eluting component), and Isomer D of compound Int-20j (4^{th} eluting component). Isomer A, Isomer C, and Isomer D were each further purified using the chiral preparative SFC conditions described above to afford sufficient purity. LCMS anal. calcd. for C₂₃H₂₅F₂N₃O₆: 477.17; Found: 478.17 (M+1)⁺.

### Step K-Synthesis of Compound 57, Compound 58, Compound 59, and Compound 60

Isomer A of compound Int-20j (11.0 mg, 0.023 mmol), magnesium bromide (45.9 mg, 0.249 mmol), and acetonitrile (0.5 mL) were combined and stirred at room temperature. After 2 hours, the reaction mixture was diluted with MeOH and filtered (0.45 μm syringe filter) before being purified by reverse phase HPLC (Waters Sunfire C18 OBD, 10 μm, 30×150 mm column) eluting with 10-90% (ACN/water)+0.05% TFA. Product fractions were combined and concentrated under reduced pressure until most of the ACN had been removed. The remaining aqueous solution was extracted with DCM (3×˜5 mL). The organic layers were sequentially dried over Na₂SO₄, filtered, combined, and evaporated under reduced pressure. The resulting residue was dissolved in ACN (˜5 mL), diluted with water (5 mL), frozen, and lyophilized to afford compound 57. ¹H NMR (500 MHz, CD₃OD): δ 7.50-7.43 (m, 1H); 7.02-6.92 (m, 2H); 5.66 (t, J=7.8 Hz, 1H); 4.69-4.61 (m, 2H); 3.96 (d, J=13.4 Hz, 1H); 3.89 (d, J=13.3 Hz, 1H); 3.71 (dq, J=14.4, 7.1 Hz, 1H); 3.61-3.49 (m, 3H); 3.26 (s, 3H); 2.93 (dd, J=12.8, 7.8 Hz, 1H); 2.07 (dd, J=12.8, 7.9 Hz, 1H); 1.24 (t, J=7.2 Hz, 3H). LCMS anal. calcd. for C₂₂H₂₃F₂N₃O₆: 463.16; Found: 464.35 (M+1)⁺.

Following essentially the method employed to produce compound 57 in step K of example 20, compound 58 was prepared from Isomer B of compound Int-20j. ¹H NMR (500 MHz, CD₃OD): δ 7.50-7.43 (m, 1H); 7.01-6.91 (m, 2H); 5.74 (d, J=7.5 Hz, 1H); 4.69-4.60 (m, 2H); 4.00 (d, J=13.0 Hz, 1H); 3.84-3.72 (m, 2H); 3.69 (d, J=9.1 Hz, 1H); 3.64 (d, J 9.1 Hz, 1H); 3.44 (dq, J=14.1, 7.1 Hz, 1H); 3.38 (s, 3H); 2.49 (d, J=13.9 Hz, 1H); 2.32 (dd, J 13.5, 7.7 Hz, 1H); 1.25 (t, J=7.2 Hz, 3H). LCMS anal. calcd. for C₂₂H₂₃F₂N₃O₆: 463.16; Found: 464.13 (M+1)⁺.

Following essentially the method employed to produce compound 57 in step K of example 20, compound 59 was prepared from Isomer C of compound Int-20j. ¹H NMR (500 MHz, CD₃OD): δ 7.50-7.43 (m, 1H); 7.02-6.92 (m, 2H); 5.66 (t, J=7.8 Hz, 1H); 4.69-4.61 (m, 2H); 3.96 (d, J=13.4 Hz, 1H); 3.89 (d, J=13.3 Hz, 1H); 3.71 (dq, J=14.3, 7.2 Hz, 1H); 3.61-3.49 (m, 3H); 3.26 (s, 3H); 2.93 (dd, J=12.8, 7.7 Hz, 1H); 2.07 (dd, J=12.8, 7.8 Hz, 1H); 1.24 (t, J=7.2 Hz, 3H). LCMS anal. calcd. for C₂₂H₂₃F₂N₃O₆: 463.16; Found: 464.18 (M+1)⁺.

Following essentially the method employed to produce compound 57 in step K of example 20, compound 60 was prepared from Isomer D of compound Int-20j. ¹H NMR (500 MHz, CD₃OD): δ 7.49-7.42 (m, 1H); 7.01-6.91 (m, 2H); 5.74 (d, J=7.5 Hz, 1H); 4.68-4.60 (m, 2H); 4.00 (d, J=13.0 Hz, 1H); 3.84-3.72 (m, 2H); 3.69 (d, J=9.0 Hz, 1H); 3.64 (d, J 9.1 Hz, 1H); 3.44 (dq, J=14.3, 7.3 Hz, 1H); 3.38 (s, 3H); 2.48 (d, J=13.9 Hz, 1H); 2.32 (dd, J 13.5, 7.8 Hz, 1H); 1.25 (t, J=7.2 Hz, 3H). LCMS anal. calcd. for C₂₂H₂₃F₂N₃O₆: 463.16; Found: 464.19 (M+1)⁺.

### Example 21

### Preparation of Compounds 61-64

Starting from Int-20i, using essentially the same method described in Step J and Step K in example 20 with the exception of substituting with 2,4,6-trifluorobenzylamine, purifying by chiral preparative SFC (ChiralPak AS-H, 21×250 mm column, 50 g/min, 120 bar, 30% EtOH/CO₂, 40° C.) to afford a mixture of Isomer A and Isomer B, Isomer C, and Isomer D, further purifying the mixture of Isomer A and Isomer B by chiral preparative SFC (ChiralPak OJ-H, 21×250 mm column two times, 50 g/min, 120 bar, 15% EtOH/CO₂, 40° C.) to afford Isomer A and Isomer B, and further purifying Isomer by chiral preparative SFC (ChiralPak OJ-H, 21×250 mm column two times, 50 g/m, 120 bar, 15% EtOH/CO₂, 40° C.) in step J, the following compounds were prepared:

| | | MS | |
|---|---|---|---|
| Compound # | Structure | (M + H) | ¹H NMR |
| 61 (Isomer A) | 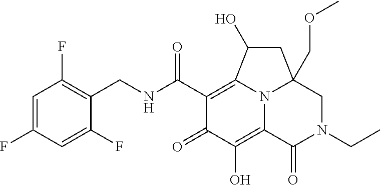 [SMILES: CCN1CC2(COC)CC(O)c3c(C(=O)NCc4c(F)cc(F)cc4F)c(=O)c(O)c(n32)C1=O] | 482.21 | (500 MHz, CD₃OD): δ 6.99-6.83 (m, 2H); 5.74 (d, J = 7.6 Hz, 1H); 4.71-4.63 (m, 2H); 3.99 (d, J = 13.0 Hz, 1H); 3.84-3.71 (m, 2H); 3.68 (d, J = 9.6 Hz, 1H); 3.63 (d, J = 9.1 Hz, 1H); 3.43 (dq, J = 14.4, 7.2 Hz, 1H); 3.37 (s, 3H); 2.48 (d, J = 13.9 Hz, 1H); 2.31 (dd, J = 13.7, 7.6 Hz, 1H); 1.24 (t, J = 7.2 Hz, 3H). |
| 62 (Isomer B) | 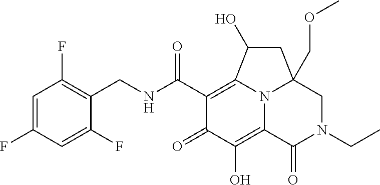 [SMILES: CCN1CC2(COC)CC(O)c3c(C(=O)NCc4c(F)cc(F)cc4F)c(=O)c(O)c(n32)C1=O] | 482.20 | (500 MHz, CD₃OD): δ 6.95-6.87 (m, 2H); 5.66 (t, J = 7.8 Hz, 1H); 4.73- 4.64 (m, 2H); 3.95 (d, J = 13.3 Hz, 1H); 3.89 (d, J = 13.3 Hz, 1H); 3.70 (dq, J = 14.2, 7.2 Hz, 1H); 3.61-3.48 (m, 3H); 3.25 (s, 3H); 2.93 (dd, J = 12.8, 7.8 Hz, 1H); 2.06 (dd, J = 12.7, 7.9 Hz, 1H); 1.24 (t, J = 7.2 Hz, 3H). |
| 63 (Isomer C) | 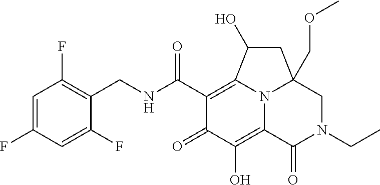 [SMILES: CCN1CC2(COC)CC(O)c3c(C(=O)NCc4c(F)cc(F)cc4F)c(=O)c(O)c(n32)C1=O] | 482.20 | (500 MHz, CD₃OD): δ 6.95-6.87 (m, 2H): 5.66 (t, J = 7.8 Hz, 1H); 4.73- 4.64 (m, 2H); 3.95 (d, J = 13.4 Hz, 1H); 3.89 (d, J = 13.4 Hz, 1H); 3.70 (dq, J = 14.4, 7.1 Hz, 1H); 3.61-3.48 (m, 3H); 3.26 (s, 3H); 2.93 (dd, J = 12.8, 7.8 Hz, 1H); 2.06 (dd, J = 12.8, 7.9 Hz, 1H); 1.24 (t, J = 7.2 Hz, 3H). |
| 64 (Isomer D) | 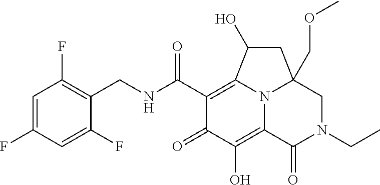 [SMILES: CCN1CC2(COC)CC(O)c3c(C(=O)NCc4c(F)cc(F)cc4F)c(=O)c(O)c(n32)C1=O] | 482.21 | (500 MHz, CD₃OD): δ 6.95-6.87 (m, 2H); 5.74 (d, J = 7.6 Hz, 1H); 4.72-4.63 (m, 2H); 3.99 (d, J = 13.0 Hz, 1H); 3.84-3.71 (m, 2H); 3.71- 3.66 (m, 1H); 3.63 (d, J = 9.1 Hz, 1H); 3.43 (dq, J = 14.4, 7.3 Hz, 1H); 3.37 (s, 3H); 2.48 (d, J = 13.9 Hz, 1H); 2.31 (dd, J = 13.8, 7.8 Hz, 1H); 1.24 (t, J = 7.2 Hz, 3H). |

### Example 22

### Preparation of Compounds 65-72

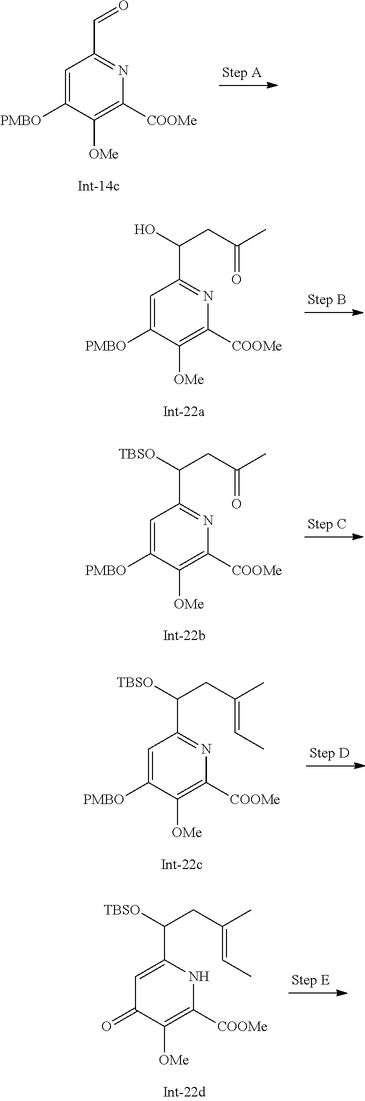

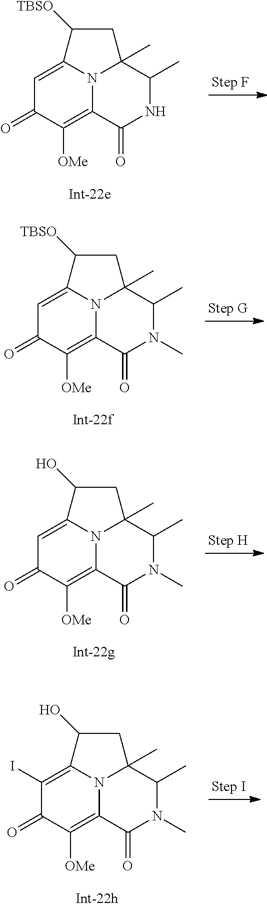

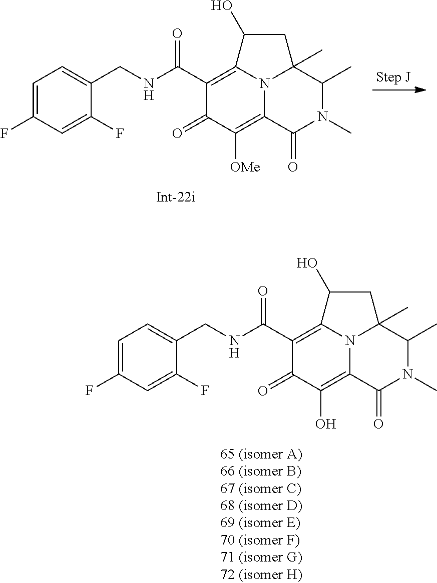

### Step A Synthesis of Compound Int-22a

A mixture of compound Int-14c (50 mg, 0.151 mmol) and K₂CO₃ (83 mg, 0.604 mmol) in dry acetone (5 mL) was stirred at 70° C. After 14 hours, the reaction mixture was filtered and the filtrate was evaporated to afford compound Int-22a. This material was used in step B of example 22 without further purification. LCMS anal. calcd. for C₂₀H₂₃NO₇: 389.2; Found: 390.1 (M+1)⁺.

### Step B—Synthesis of Compound Int-22b

To a solution of compound Int-22a (60 mg, 0.154 mmol), DMAP (9.41 mg, 0.077 mmol), and 2,6-dimethylpyridine (165 mg, 1.541 mmol) in DCM (5 mL) was added tert-butyldimethylsilyl trifluoromethanesulfonate (244 mg, 0.925 mmol) dropwise at 0° C. under N₂. The mixture was stirred at 25° C. for 1 hour before being quenched with water (10 mL). The separated aqueous phase was extracted with DCM (2×10 m). The combined organic layers were dried over Na₂SO₄, filtered, and concentrated. The residue was purified by flash silica gel chromatography (4 g column) eluting with 0-25% EtOAc/petroleum ether to afford compound Int-22b. LCMS anal. calcd. for C₂₆H₃₇NO₇Si: 503.2; Found: 504.4 (M+1)⁺.

### Step C—Synthesis of Compound Int-22c

To a solution of compound Int-22b (6.2 g, 12.31 mmol) and 5-(ethylsulfonyl)-1-phenyl-1H-tetrazole (5.87 g, 24.62 mmol) in THF (120 mL) was added LiHMDS 1 M in THF (49.2 mL, 49.2 mmol) dropwise at −78° C. under N₂. Then the reaction mixture was stirred at −78° C. for 1 hour before being quenched with aqueous NH₄Cl (200 mL) at −78° C. The aqueous layer was extracted with EtOAc (3×100 mL). The combined organic layers were dried over Na₂SO₄, filtered, and concentrated. The residue was purified by flash silica gel chromatography (120 g column) eluting with 0-8% EtOAc/petroleum ether to afford compound Int-22c. LCMS anal. calcd. for C₂₈H₄₁NO₆Si: 515.3; Found: 516.3 (M+1)⁺.

### Step D—Synthesis of Compound Int-22d

A mixture of compound Int-22c (2.45 g, 4.75 mmol) in DCM (50 mL) and TFA (5 mL) was stirred at 25° C. After 1 hour, the solvent was evaporated and the residue was purified by flash silica gel chromatography (40 g column) eluting with 0-50% EtOAc/petroleum ether to afford compound Int-22d. LCMS anal. calcd. for C₂₀H₃₃NO₅Si: 395.2; Found: 396.2 (M+1)⁺.

### Step E—Synthesis of Compound Int-22e

To a mixture of compound Int-22d (1.7 g, 4.30 mmol) and O-(2,4-dinitrophenyl)hydroxylamine (2.57 g, 12.89 mmol) in CF₃CH₂OH (20 mL) was added bis[rhodium(α,α,α′,α′-tetramethyl-1,3-benzenedipropionic acid)] (0.164 g, 0.215 mmol). The mixture was stirred at 60° C. under N₂. After 36 hours, the solvent was evaporated and the residue was purified by flash silica gel chromatography (40 g column) eluting with 0-5% MeOH/DCM to afford compound Int-22e. LCMS anal. calcd. for C₁₉H₃₀N₂O₄Si: 378.2; Found: 379.1 (M+1)⁺.

### Step F—Synthesis of Compound Int-22f

To a mixture of compound Int-22e (1 g, 2.64 mmol) and Mel (0.496 mL, 7.93 mmol) in DMF (10 mL) was added NaH (0.211 g, 5.28 mmol) at 0° C. under N₂. The mixture was stirred at 0° C. for 1 hour before being quenched with aqueous NH₄Cl (2 mL). The mixture was purified by preparative reverse phase HPLC (Phenomenex Synergi C18, 4 μm, 30×150 mm column) eluting with 34-44% ACN/(water+0.1% TFA) to afford a mixture of Isomer A, Isomer B, Isomer C, and Isomer D of compound Int-22f (1^{st} eluting component), a mixture of Isomer E and Isomer F of compound Int-22f (2^{nd} eluting component), and a mixture of Isomer G and Isomer H of compound Int-22f (3^{rd} eluting component). LCMS anal. calcd. for C₂₀H₃₂N₂O₄Si: 392.2; Found: 393.2 (M+1)⁺.

### Step G—Synthesis of Compound Int-22g

A mixture of Isomer A, Isomer B, Isomer C, and Isomer D of compound Int-22f (270 mg, 0.688 mmol) and TBAF 1 M in THF (0.344 mL, 0.344 mmol) in THF (5 mL) was stirred at 25° C. After 14 hours, the solvent was evaporated to dryness and the residue was purified by flash silica gel chromatography (12 g column) eluting with 0-12% MeOH/DCM to afford a mixture of Isomer A, Isomer B, Isomer C, and Isomer D of compound Int-22g. LCMS anal. calcd. for C₁₄H₁₈N₂O₄: 278.1; Found: 279.1 (M+1)⁺.

To a solution of Isomer E and Isomer F of compound Int-22f (120 mg, 0.306 mmol) in THF (5 mL) was added a solution of 1 M TBAF in THF (0.611 mL, 0.611 mmol). The reaction was stirred at 25° C. for 1 hour. The solvent was evaporated to dryness and the residue was purified by flash silica gel chromatography (4 g column) eluting with 0-10% MeOH/DCM to afford a mixture of Isomer E and Isomer F of compound Int-22g. LCMS anal. calcd. for C₁₄H₁₈N₂O₄: 278.1; Found: 279.1 (M+1)⁺.

A mixture of Isomer G and Isomer H of compound Int-22f (400 mg, 1.019 mmol) and TBAF 1 M in THF (3.06 mL, 3.06 mmol) in THF (5 mL) was stirred at 25° C. After 14 hours, the solvent was evaporated to dryness and the residue was purified by flash silica gel chromatography (12 g column) eluting with 0-10% MeOH/DCM to afford a mixture of Isomer G and Isomer H of compound Int-22g. LCMS anal. calcd. for C₁₄H₁₈N₂O₄: 278.1; Found: 279.1 (M+1)⁺.

### Step H—Synthesis of Compound Int-22h

A mixture of Isomer A, Isomer B, Isomer C, and Isomer D of compound Int-22g (0.25 g, 0.898 mmol), m-CPBA (0.620 g, 3.59 mmol), and NIS (0.808 g, 3.59 mmol) in MeOH (10 mL) was stirred at 90° C. After 1 hour, the reaction was quenched with 2 g of Na₂S₂O₅ and 0.5 mL of water. The mixture was stirred at 25° C. for 10 minutes before the solvent was evaporated and the residue was purified by flash silica gel chromatography (12 g column) eluting with 0-10% MeOH/DCM to afford a mixture of Isomer A, Isomer B, Isomer C, and Isomer D of compound Int-22h. LCMS anal. calcd. for C₁₄H₁₇IN₂O₄: 404.0; Found: 405.0 (M+1)⁺.

A mixture of Isomer E and Isomer F of compound Int-22g (70 mg, 0.252 mmol), m-CPBA (130 mg, 0.755 mmol), and NIS (170 mg, 0.755 mmol) in MeOH (10 mL) was stirred at 90° C. After 1 hour, the reaction was quenched with 300 mg of Na₂S₂O₅ and 1 mL of water. The mixture was stirred at 25° C. for 10 minutes before the solvent was evaporated and the residue was purified by flash silica gel chromatography (4 g column) eluting with 0-12% MeOH/DCM to afford a mixture of Isomer E and Isomer F of compound Int-22h. LCMS anal. calcd. for C₁₄H₁₇IN₂O₄: 404.0; Found: 405.1 (M+1)⁺.

A mixture of Isomer G and Isomer H of compound Int-22g (160 mg, 0.575 mmol), m-CPBA (298 mg, 1.725 mmol), and NIS (388 mg, 1.725 mmol) in MeOH (10 mL) was stirred at 90° C. After 2 hours, the reaction was quenched with 700 mg of Na₂S₂O₅ and 1 mL of water. The mixture was stirred at 25° C. for 10 minutes before the solvent was evaporated and the residue was purified by flash silica gel chromatography (12 g column) eluting with 0-10% MeOH/DCM to afford a mixture of Isomer G and Isomer H of compound Int-22h. LCMS anal. calcd. for C₁₄H₁₇IN₂O₄: 404.0; Found: 405.1 (M+1)⁺.

### Step I—Synthesis of Compound Int-22i

To the solution of a mixture of Isomer A, Isomer B, Isomer C, and Isomer D of compound Int-22h (370 mg, 0.915 mmol) in DMSO (3 mL) was added (2,4-difluorophenyl)methanamine (393 mg, 2.75 mmol), DIEA (0.959 mL, 5.49 mmol), and Pd(Ph₃P)₄ (529 mg, 0.458 mmol). The mixture was degassed and purged with CO three times. The resulting mixture was stirred at 90° C. under CO (15 psi). After 3 hours, the mixture was filtered and purified by preparative reverse phase HPLC (Boston Green ODS, 5 μm, 30×150 mm column) eluting with 29-49% ACN/(water+0.1% TFA), which was further purified by preparative SFC (DAICEL CHIRALPAK AD-H, 5 μm, 30×250 mm column, 50 mL/min, 35% (IPA+0.1% NH₃H₂O)/CO₂) to afford Isomer A of compound Int-22i (1st eluting component), Isomer B of compound Int-22i (2^{nd} deluting component), Isomer C of compound Int-22i (3^{rd} eluting component), and Isomer D of compound Int-22i (1^{st} eluting component). LCMS anal. calcd. for C₂₂H₂₃F₂N₃O₅: 447.2; Found: 448.2 (M+1)⁺.

To a solution of a mixture of Isomer E and Isomer F of compound Int-22h (110 mg, 0.272 mmol) in DMSO (10 mL) was added (2,4-difluorophenyl)methanamine (117 mg, 0.816 mmol), DIEA (0.285 mL, 1.633 mmol), and Pd(Ph₃P)₄ (157 mg, 0.136 mmol). The mixture was degassed and purged with CO three times. The resulting mixture was stirred at 90° C. under CO (15 psi). After 2 hours, the mixture was filtered and purified by preparative reverse phase HPLC (Boston Green ODS, 5 μm, 30×150 mm column) eluting with 29-49% ACN/(water+0.1% TFA), which was further purified by preparative SFC (DAICEL CHIRALPAK AD, 10 μm, 50×250 mm column, 70 mL/min, 40% (IPA+0.1% NH₃H₂O)/CO₂) to afford Isomer E of compound Int-22i (1^{st} eluting component) and Isomer F of compound Int-22i (2^{nd} eluting component). LCMS anal. calcd. for C₂₂H₂₃F₂N₃O₅: 447.2; Found: 448.2 (M+1)⁺.

To a solution of a mixture of Isomer G and Isomer H of compound Int-22h (320 mg, 0.792 mmol) in DMSO (10 mL) was added (2,4-difluorophenyl)methanamine (340 mg, 2.375 mmol), DIEA (0.830 mL, 4.75 mmol), and Pd(Ph₃P)₄ (457 mg, 0.396 mmol). The mixture was degassed and purged with CO three times. The resulting mixture was stirred at 90° C. under CO (15 psi). After 3 hours, the mixture was filtered and purified by preparative reverse phase HPLC (Boston Green ODS, 5 μm, 30×150 mm column) eluting with 29-49% ACN/(water+0.1% TFA), which was further purified by preparative SFC (DAICEL CHIRALPAK AD, 10 μm, 50×250 mm column, 70 mL/min, 50% (IPA+0.1% NH₃H₂O)/CO₂) to afford Isomer G of compound Int-22i (1^{st} eluting component) and Isomer H of compound Int-22i (2^{nd} eluting component). LCMS anal. calcd. for C₂₂H₂₃F₂N₃O₅: 447.2; Found: 448.2 (M+1)⁺.

### Step J—Synthesis of Compound 65, Compound 66, Compound 67, Compound 68, Compound 69, Compound 70, Compound 71, and Compound 72

A mixture of Isomer A of compound Int-22i (7 mg, 0.016 mmol) and magnesium bromide (28.8 mg, 0.156 mmol) in acetonitrile (3 mL) was stirred at 25° C. After 2 hours, MeOH (1 mL) was added and the mixture was purified by preparative HPLC (Boston Green ODS, 5 μm, 30×150 mm column) eluting with 30-60% ACN/(water+0.1% TFA) to afford compound 65. ¹H NMR (400 MHz, CD₃OD) δ:11.34 (br s, 1H); 7.60-7.25 (m, 1H); 7.06-6.75 (m, 2H); 5.67 (t, J=7.9 Hz, 1H); 4.72-4.53 (m, 2H); 3.91 (q, J=6.6 Hz, 1H); 3.23-3.11 (m, 3H); 2.67 (dd, J=13.0, 7.8 Hz, 1H); 2.26 (dd, J=13.0, 8.1 Hz, 1H); 1.59-1.37 (m, 3H); 1.26 (d, J=6.6 Hz, 3H). LCMS anal. calcd. for C₂₁H₂F₂N₃O₅: 433.1; Found: 434.2 (M+1)⁺.

Following essentially the method employed to produce compound 65 in step J of example 22, compound 66 was prepared from Isomer B of compound Int-22i. ¹H NMR (400 MHz, CD₃OD) δ: 7.52-7.34 (m, 1H); 7.02-6.83 (m, 2H); 5.64 (t, J=8.1 Hz, 1H); 4.73-4.53 (m, 2H); 4.06 (q, J=7.0 Hz, 1H); 3.14 (s, 3H); 2.80 (dd, J=12.5, 7.3 Hz, 1H); 2.13-1.98 (m, 1H); 1.45 (d, J=7.1 Hz, 3H); 1.28 (s, 3H). LCMS anal. calcd. for C₂₁H₂F₂N₃O₅: 433.1; Found: 434.2 (M+1)⁺.

Following essentially the method employed to produce compound 65 in step J of example 22, compound 67 was prepared from Isomer C of compound Int-22i. ¹H NMR (400 MHz, CD₃OD) δ: 11.34 (br s, 1H); 7.53-7.31 (m, 1H); 7.06-6.78 (m, 2H); 5.67 (t, J=7.9 Hz, 1H); 4.72-4.53 (m, 2H); 3.96-3.78 (m, 1H); 3.23-3.12 (m, 3H); 2.67 (dd, J=13.0, 7.8 Hz, 1H); 2.32-2.19 (m, 1H); 1.51-1.41 (m, 3H); 1.26 (br d, J=6.6 Hz, 3H). LCMS anal. calcd. for C₂₁H₂F₂N₃O₅: 433.1; Found: 434.2 (M+1)⁺.

Following essentially the method employed to produce compound 65 in step J of example 22, compound 68 was prepared from Isomer D of compound Int-22i. ¹H NMR (400 MHz, CD₃OD) δ: 7.55-7.33 (m, 1H); 7.00-6.79 (m, 2H); 5.63 (t, J=8.1 Hz, 1H); 4.75-4.45 (m, 2H); 4.07 (q, J=6.8 Hz, 1H); 3.13 (s, 3H); 2.79 (dd, J=12.5, 7.6 Hz, 1H); 2.06 (br dd, J=12.1, 8.9 Hz, 1H); 1.44 (d, J=6.8 Hz, 3H); 1.27 (s, 3H). LCMS anal. calcd. for C₂₁H₂F₂N₃O₅: 433.1; Found: 434.2 (M+1)⁺.

Following essentially the method employed to produce compound 65 in step J of example 22, compound 69 was prepared from Isomer E of compound Int-22i. ¹H NMR (400 MHz, CD₃OD) δ: 10.90 (br s, 1H); 7.53-7.28 (m, 1H); 7.03-6.75 (m, 2H); 5.75 (d, J=7.8 Hz, 1H); 4.63 (s, 2H); 3.92 (m, 1H); 3.18 (s, 3H); 2.61 (dd, J=13.8, 7.5 Hz, 1H); 2.16 (d, J=14.2 Hz, 1H); 1.70 (s, 3H); 1.17 (d, J=6.6 Hz, 3H). LCMS anal. calcd. for C₂₁H₂₁F₂N₃O₅: 433.1; Found: 434.2 (M+1)⁺.

Following essentially the method employed to produce compound 65 in step J of example 22, compound 70 was prepared from Isomer F of compound Int-22i. ¹H NMR (400 MHz, CD₃OD) δ: 10.90 (br s, 1H); 7.57-7.31 (m, 1H); 7.06-6.78 (m, 2H); 5.75 (d, J=7.8 Hz, 1H); 4.62 (s, 2H); 3.92 (q, J=6.6 Hz, 1H); 3.18 (s, 3H); 2.61 (dd, J=14.1, 7.7 Hz, 1H); 2.16 (d, J 13.9 Hz, 1H); 1.70 (s, 3H); 1.17 (d, J=6.6 Hz, 3H). LCMS anal. calcd. for C₂₁H₂F₂N₃O₅: 433.1; Found: 434.2 (M+1)⁺.

Following essentially the method employed to produce compound 65 in step J of example 22, compound 71 was prepared from Isomer G of compound Int-22i. ¹H NMR (400 MHz, CD₃OD) δ: 7.50-7.34 (m, 1H); 7.04-6.83 (m, 2H); 5.66 (d, J=7.3 Hz, 1H); 4.68-4.54 (m, 2H); 3.96 (m, 1H); 3.13 (s, 3H); 2.40-2.24 (m, 2H); 1.48 (s, 3H); 1.45 (d, J=6.8 Hz, 3H). LCMS anal. calcd. for C₂₁H₂₁F₂N₃O₅: 433.1; Found: 434.2 (M+1)⁺.

Following essentially the method employed to produce compound 65 in step J of example 22, compound 72 was prepared from Isomer H of compound Int-22i. ¹H NMR (400 MHz, CD₃OD) δ: 7.52-7.36 (m, 1H); 7.03-6.82 (m, 2H); 5.66 (d, J=7.1 Hz, 1H); 4.71-4.49 (m, 2H); 3.96 (q, J=6.8 Hz, 1H); 3.13 (s, 3H); 2.46-2.18 (m, 2H); 1.48 (s, 3H); 1.45 (d, J=6.8 Hz, 3H). LCMS anal. calcd. for C₂₁H₂₁F₂N₃O₅: 433.1; Found: 434.2 (M+1)⁺.

### Example 23

### Preparation of Compound Int-23b

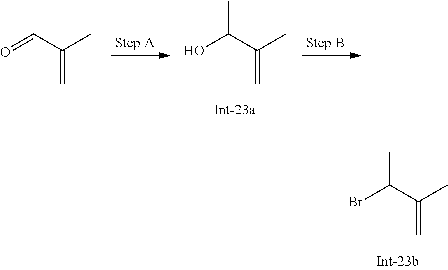

### Step A—Synthesis of Compound Int-23a

To a solution of methylmagnesium bromide 3 M in Et₂O (71.1 mL, 213 mmol) stirred at 0° C. was added a solution of methacrylaldehyde (13.6 g, 194 mmol) in Et₂O (130 mL) dropwise during a period of 45 mins under N₂. After the addition, the solution was stirred at 0° C. for 30 minutes before being poured into 200 mL of 2 N HCl at 0° C. The layers were separated and the aqueous layer was extracted with Et₂O (2×200 mL). The combined organic extracts were washed with NaHCO₃ (150 mL) and brine (150 mL), dried over Na₂SO₄, and filtered. The filtrate was concentrated at room temperature to leave a residue which was distilled at reduced pressure (water pump, 55 70° C.) to afford compound Int-23a. ¹H NMR (400 MHz, CDCl₃) δ: 4.93 (s, 1H); 4.77 (s, 1H); 4.22 (q, J=6.3 Hz, 1H); 1.73 (s, 3H); 1.26 (d, J=6.6 Hz, 3H).

### Step B—Synthesis of Compound Int-23b

To a 0° C. solution of compound Int-23a (3 g, 34.8 mmol) in Et₂O (50 mL) was added phosphorus tribromide (1.314 mL, 13.93 mmol) dropwise under N₂ with vigorous stirring. After 1 hour at 0° C., the reaction was quenched with 20 mL of water. The layers were separated, and the organic extract was washed with aqueous NaHCO₃ (30 mL) and water (30 mL), dried over Na₂SO₄, and filtered. The filtrate was concentrated at room temperature to give compound Int-23b that was used in Step C of example 14 without further purification. ¹H NMR (400 MHz, CDCl₃) δ: 5.06 (s, 1H); 4.86 (t, J=1.3 Hz, 1H); 4.72 (q, J=6.8 Hz, 1H); 1.88 (s. 3H); 1.62 (d, J=6.8 Hz, 3H).

### Example 24

### Preparation of Compounds 73-80

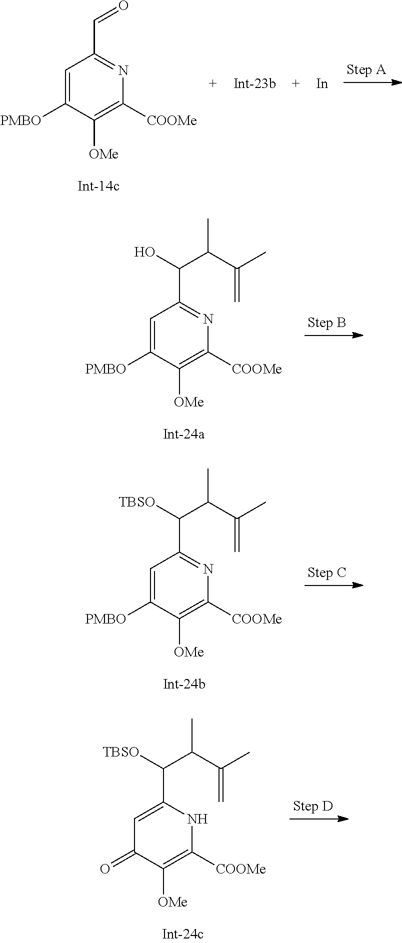

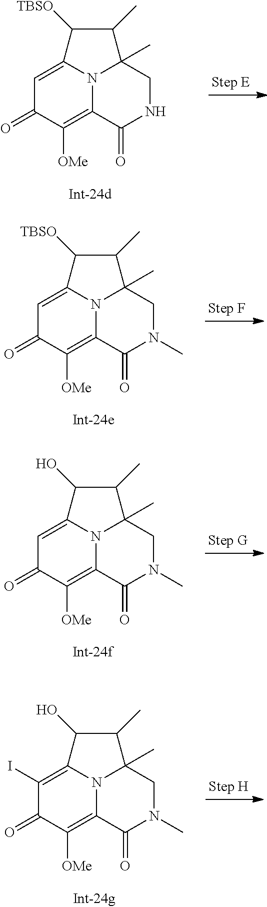

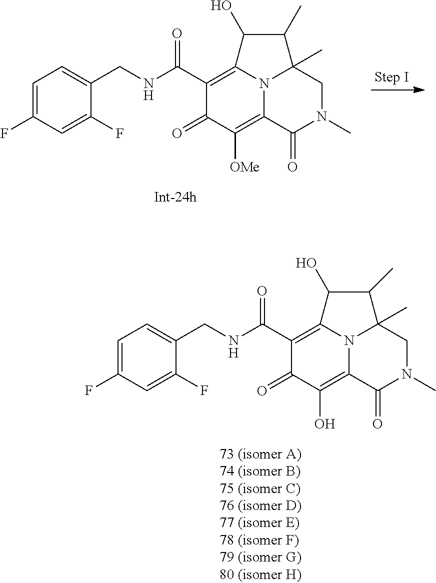

### Step A Synthesis of Compound Int-24a

The mixture of compound Int-14c (1 g, 3.02 mmol), sodium iodide (0.905 g, 6.04 mmol) and indium (1.733 g, 15.09 mmol) in DMF (15 mL) was stirred at 25° C. for 10 minutes before Int-23b (1.349 g, 9.05 mmol) was added. The mixture was stirred at 25° C. for 14 hours before being diluted with EtOAc (50 mL). After filtration, the organic phase was washed with water (2×20 mL) and brine (20 mL) before being dried over Na₂SO₄. After filtration, the organic solvent was removed in vacuo and the residue was purified by flash silica gel chromatography (20 g column) eluting with 0-35% EtOAc/petroleum ether to afford compound Int-24a. LCMS anal. calcd. for C₂₂H₂₇NO₆: 401.2; Found: 402.2 (M+1)⁺.

### Step B Synthesis of Compound Int-24b

To a 0° C. solution of compound Int-24a (800 mg, 1.993 mmol), DMAP (122 mg, 0.996 mmol), and 2,6-dimethylpyridine (2135 mg, 19.93 mmol) in DCM (10 mL) was added tert-butyldimethylsilyl trifluoromethanesulfonate (3161 mg, 11.96 mmol) dropwise. The mixture was stirred at 25° C. for 1 hour before being quenched with water (20 mL). The separated aqueous phase was extracted with DCM (2×20 mL), and the combined organic layers were dried over Na₂SO₄, filtered, and concentrated to dryness. The resulting residue was purified by flash silica gel chromatography (40 g column) eluting with 0-25% EtOAc/petroleum ether to afford compound Int-24b. LCMS anal. calcd. for C₂H₄₁NO₆Si: 515.3; Found: 516.3 (M+1)⁺.

### Step C—Synthesis of Compound Int-24c

The mixture of compound Int-24b (750 mg, 1.454 mmol) in DCM (10 mL) and TFA (1 mL) was stirred at 25° C. After 2 hours, the mixture was concentrated in vacuo and the residue was purified by flash silica gel chromatography (12 g column) eluting with 0-50% EtOAc/petroleum ether to afford compound Int-24c. LCMS anal. calcd. for C₂₀H₃₃NO₅Si: 395.2; Found: 396.2 (M+1)⁺.

### Step D—Synthesis of Compound Int-24d

To the mixture of compound Int-24c (640 mg, 1.618 mmol) and O-(2,4-dinitrophenyl)hydroxylamine (966 mg, 4.85 mmol) in CF₃CH₂OH (10 mL) was added bis[rhodium(α,α,α′,α′-tetramethyl-1,3-benzenedipropionic acid)] (24.67 mg, 0.032 mmol). The mixture was stirred at 60° C. under N₂. After 36 hours, the solvent was evaporated and the residue was purified by flash silica gel chromatography (24 g column) eluting with 0-3% MeOH/DCM to afford compound Int-24d. LCMS anal. calcd. for C₁₉H₃₀N₂O₄Si: 378.2; Found: 379.2 (M+1)⁺.

### Step E—Synthesis of Compound Int-24e

To a 0° C. mixture of compound Int-24d (340 mg, 0.898 mmol) and Mel (0.168 mL, 2.69 mmol) in DMF (5 mL) was added NaH (71.8 mg, 1.796 mmol) under N₂. The mixture was stirred at 0° C. for 1 hour before being quenched with aqueous NH₄Cl (20 mL). The aqueous layer was extracted with EtOAc (3×20 mL). The combined organic layers were washed with brine (3×10 mL), dried over Na₂SO₄, filtered, and evaporated to dryness. The resulting residue was purified by flash silica gel chromatography (4 g column) eluting with 0-8% MeOH/DCM to afford compound Int-24e. LCMS anal. calcd. for C₂H₃₂N₂O₄Si: 392.2; Found: 393.2 (M+1)⁺.

### Step F—Synthesis of Compound Int-24f

A mixture of compound Int-24e (270 mg, 0.688 mmol) and TBAF 1 M in THF (1.376 mL, 1.376 mmol) in THF (5 mL) was stirred at 25° C. After 1 hour, the solvent was evaporated to dryness and the residue was purified by flash silica gel chromatography (12 g column) eluting with 0-15% MeOH/DCM to afford compound Int-24f. LCMS anal. calcd. for C₁₄H₁₈N₂O₄: 278.1; Found: 279.1 (M+1)⁺.

### Step G—Synthesis of Compound Int-24g

A mixture of compound Int-24f (210 mg, 0.755 mmol), m-CPBA (521 mg, 3.02 mmol), and NIS (679 mg, 3.02 mmol) in MeOH (5 mL) was stirred at 90° C. After 1 hour, the reaction was quenched with 1 g of Na₂S₂O₅ and 5 mL of water. The mixture was stirred at 25° C. for 10 minutes before the solvent was evaporated and the residue was purified by flash silica gel chromatography (20 g column) eluting with 0-10% MeOH/DCM to afford compound Int-24g. LCMS anal. calcd. for C₁₄H₁₇IN₂O₄: 404.0; Found: 405.0 (M+1)⁺.

### Step H—Synthesis of Compound Int-24h

To a solution of compound Int-24g (580 mg, 1.435 mmol) in DMSO (10 mL) was added (2,4-difluorophenyl)methanamine (616 mg, 4.30 mmol), DIEA (1.504 mL, 8.61 mmol), and Pd(Ph₃P)₄ (829 mg, 0.717 mmol). The mixture was degassed and purged with CO three times. The resulting mixture was stirred at 90° C. under CO (15 psi). After 6 hours, the reaction mixture was diluted with EtOAc (30 mL), and washed with water (2×10 mL) and brine (1×10 mL). The organic layer was dried over sodium sulfate, filtered, and concentrated in vacuo. The residue was purified by flash silica gel chromatography (12 g column) eluting with 50-100% EtOAc/petroleum ether to afford the crude product, which was further purified by preparative reverse phase HPLC (Phenomenex Synergi C18, 4 μm, 30×150 mm column) eluting with 26-41% ACN/(water+0.1% TFA) to afford a mixture of Isomer A and Isomer B of compound Int-24h (1st eluting component) and a mixture of Isomer C, Isomer D, Isomer E, Isomer F, Isomer G, and Isomer H of compound Int-24h (2^{nd} eluting component).

The mixture of Isomer A and Isomer B of compound Int-24h was further purified by preparative SFC (Phenomenex-Cellulose-2, 10 μm, 30×250 mm column, 80 mL/min, 50% (EtOH+0.1% NH₃H₂O)/CO₂) to afford Isomer A of compound Int-24h (1st eluting component) and Isomer B of compound Int-24h (2^{nd} eluting component).
The mixture of Isomer C, Isomer D, Isomer E, Isomer f, Isomer G, and Isomer H of compound Int-24h was further purified by preparative SFC (Phenomenex-Cellulose-2, 5 μm, 30×250 mm column, 50 mL/min, 40% (EtOH+0.1% NH₃H₂O)/CO₂) to afford a mixture of Isomer C, Isomer D, and Isomer E of compound Int-24h (st eluting component), Isomer F of compound Int-24h (2^{nd} eluting component), Isomer G of compound Int-24h (3^{rd} eluting component), and Isomer H of compound Int-24h (4^{th} eluting component). The mixture of Isomer C, Isomer D, and Isomer E of compound Int-24h was further purified by preparative SFC (YMC CHIRAL Amylose-C, 10 μm, 30×250 mm column, 70 mL/min, 55% (EtOH+0.1% NH₃H₂O)/CO₂) to afford Isomer C of compound Int-24h (1st eluting component), Isomer D of compound Int-24h (2^{nd} eluting component), and Isomer E of compound Int-24h (3^{rd} eluting component). Isomer G of compound Int-24h was further purified by preparative SFC (DAICEL CHIRALPAK IC, 5 μm, 30×250 mm column, 50 mL/min, 50% (EtOH+0.1% NH₃H₂O)/CO₂) to afford Isomer G of compound Int-24h. LCMS anal. calcd. for C₂₂H₂₃F₂N₃O₅: 447.2; Found: 448.1 (M+1)⁺.

### Step I—Synthesis of Compound 73, Compound 74, Compound 75, Compound 76, Compound 77, Compound 78, Compound 79, and Compound 80

A mixture of Isomer A of compound Int-24h (33 mg, 0.074 mmol) and magnesium bromide (136 mg, 0.738 mmol) in acetonitrile (3 mL) was stirred at 25° C. After 2 hours, MeOH (1 mL) was added and the mixture was purified by preparative reverse phase HPLC (Boston Green ODS, 5 μm, 30×150 mm column) eluting with 30-60% ACN/(water+0.1% TFA). The product fractions were co-evaporated with toluene (2×) to afford compound 73. ¹H NMR (400 MHz, CDCl₃) δ: 10.95 (br, 1H); 7.46-7.30 (m, 1H); 6.93-6.68 (m, 2H); 5.26 (s, 1H); 4.74-4.52 (m, 2H); 3.91 (d, J=12.7 Hz, 1H); 3.30 (d, J=12.7 Hz, 1H); 3.22 (s, 3H); 2.64 (q, J=7.7 Hz, 1H); 1.70 (s, 3H); 1.11 (d, J=7.9 Hz, 3H). LCMS anal. calcd. for C₂₁H₂F₂N₃O₅: 433.1; Found: 434.2 (M+1)⁺.

Following essentially the method employed to produce compound 73 in step I of example 24, compound 74 was prepared from Isomer B of compound Int-24h. 1H NMR (400 MHz, CDCl₃) δ: 10.95 (br, 1H); 7.45-7.29 (m, 1H), 6.88-6.68 (m, 2H); 5.26 (s, 1H); 4.73-4.48 (m, 2H); 3.91 (d, J=12.7 Hz, 1H); 3.30 (d, J=12.7 Hz, 1H); 3.22 (s, 3H); 2.64 (q, J=7.7 Hz, 1H); 1.70 (s, 3H); 1.11 (d, J=7.5 Hz, 3H). LCMS anal. calcd. for C₂₁H₂F₂N₃O₅: 433.1; Found: 434.2 (M+1)⁺.

Following essentially the method employed to produce compound 73 in step I of example 24, compound 75 was prepared from Isomer C of compound Int-24h. ¹H NMR (400 MHz, CDCl₃) δ: 11.35 (br, 1H); 7.38-7.22 (m, 1H); 7.02 (s, 1H); 6.82-6.63 (m, 2H); 5.06 (d, J=9.0 Hz, 1H); 4.71-4.38 (m, 2H); 3.76-3.63 (m, 1H); 3.39 (s, 1H); 3.15 (s, 3H); 2.37-2.27 (m, 1H); 1.32-1.24 (m, 6H). LCMS anal. calcd. for C₂₁H₂F₂N₃O₅: 433.1; Found: 434.2 (M+1)⁺.

Following essentially the method employed to produce compound 73 in step I of example 24, compound 76 was prepared from Isomer D of compound Int-24h. ¹H NMR (400 MHz, CDCl₃) δ: 11.62-11.34 (m, 2H); 7.48-7.39 (m, 1H); 7.37-7.24 (m, 1H); 6.80-6.64 (m, 2H); 5.61 (d, J=7.1 Hz, 1H); 4.64-4.47 (m, 2H); 3.92 (d, J=13.0 Hz, 1H); 3.22 (d, J=13.0 Hz, 1H); 3.16 (s, 3H); 2.88 (m, 1H); 1.40 (s, 3H); 1.02 (d, J=7.3 Hz, 3H). LCMS anal. calcd. for C₂₁H₂₁F₂N₃O₅: 433.1; Found: 434.1 (M+1)⁺.

Following essentially the method employed to produce compound 73 in step I of example 24, compound 77 was prepared from Isomer E of compound Int-24h. ¹H NMR (400 MHz, CDCl₃) δ: 11.34 (br, 1H); 7.34-7.23 (m, 1H); 7.08-6.91 (m, 1H); 6.83-6.57 (m, 2H); 5.05 (d, J 8.8 Hz, 1H); 4.72-4.47 (m, 2H); 3.73 (d, J=13.0 Hz, 1H); 3.41 (d, J=12.7 Hz, 1H); 3.15 (s, 3H); 2.39-2.23 (m, 1H); 1.37-1.20 (m, 6H). LCMS anal. calcd. for C₂₁H₂₁F₂N₃O₅: 433.1; Found: 434.2 (M+1)⁺.

Following essentially the method employed to produce compound 73 in step I of example 24, compound 78 was prepared from Isomer F of compound Int-24h. ¹H NMR (400 MHz, CDCl₃) δ: 11.36 (br, 1H); 10.80 (br, 1H); 7.34-7.23 (m, 1H); 6.87-6.65 (m, 2H); 5.38 (d, J=6.6 Hz, 1H); 4.90 (br s, 1H); 4.62-4.51 (m, 2H); 3.66 (d, J=12.5 Hz, 1H); 3.40 (d, J=12.7 Hz, 1H); 3.14 (s, 3H); 2.37-2.24 (m, 1H); 1.46 (s, 3H); 1.21 (d, J=7.1 Hz, 3H). LCMS anal. calcd. for C₂₁H₂₁F₂N₃O₅: 433.1; Found: 434.2 (M+1)⁺.

Following essentially the method employed to produce compound 73 in step I of example 24, compound 79 was prepared from Isomer G of compound Int-24h. ¹H NMR (400 MHz, CDCl₃) δ: 11.47 (br, 2H); 7.47-7.40 (m, 1H); 7.34-7.26 (m, 1H); 6.82-6.67 (m, 2H); 5.61 (d, J 6.8 Hz, 1H); 4.66-4.48 (m, 2H); 3.92 (d, J=13.0 Hz, 1H); 3.22 (d, J=12.7 Hz, 1H); 3.16 (s, 3H); 2.92-2.84 (m, 1H); 1.40 (s, 3H); 1.03 (br d, J=3.4 Hz, 3H). LCMS anal. calcd. for C₂₁H₂₁F₂N₃O₅: 433.1; Found: 434.1 (M+1)⁺.

Following essentially the method employed to produce compound 73 in step I of example 24, compound 80 was prepared from Isomer H of compound Int-24h. ¹H NMR (400 MHz, CDCl₃) δ: 11.35 (br, 1H); 10.79 (br, 1H); 7.39-7.26 (m, 1H); 6.86-6.65 (m, 2H); 5.38 (d, J=6.8 Hz, 1H); 4.90 (br s, 1H); 4.64-4.44 (m, 2H); 3.67 (d, J=12.7 Hz, 1H); 3.40 (d, J=12.7 Hz, 1H); 3.23-3.04 (m, 3H), 2.34-2.22 (m, 1H); 1.46 (s, 3H); 1.20 (d, J=7.1 Hz, 3H). LCMS anal. calcd. for C₂₁H₂₁F₂N₃O₅: 433.1; Found: 434.2 (M+1)⁺.

### Assessing Antiviral Potency in a Multiple Round HIV-1 Infection Assay

The antiviral activity of the Examples herein was assessed in an assay that measures the rate of replication of HIV in cell culture, and performed according to the following procedure. HIV-1 replication was monitored using MT4-gag-GFP clone D3 (hereafter designated MT4-GFP), which are MT-4 cells modified to harbor a GFP reporter gene, the expression of which is dependent on the HIV-1 expressed proteins tat and rev. Productive infection of an MT4-GFP cell with HIV-1 results in GFP expression approximately 24 h post-infection. MT4-GFP cells were maintained at 37° C./5% CO₂/90% relative humidity in RPMI 1640 supplemented with 10% fetal bovine serum, 100 U/ml penicillin/streptomycin, and 400 μg/ml G418 to maintain the reporter gene. For infections, MT4-GFP cells were placed in the same medium lacking G418 and infected overnight with HIV-1 (H9/IIIB strain) virus at an approximate multiplicity of infection of 0.01 in the same incubation conditions. Cells were then washed and re-suspended in either RPMI 1640 at 2×10⁵ cells/mL (0% NHS condition) or 100% normal human serum (NHS) at 2×10⁵ cells/mL (100% NHS condition). Compound plates were prepared by dispensing compounds dissolved in DMSO into wells of 384 well poly-D-lysine-coated plates (0.2 μl/well) using an ECHO acoustic dispenser. Each compound was tested in a 10-point serial 3-fold dilution (typical final concentrations: 1050 nM-0.05 nM for 0% NHS condition or 42 μM-2.13 nM for 100% NHS condition). Controls included no inhibitor (DMSO only) and a combination of three antiviral agents (efavirenz, indinavir, an in-house integrase strand transfer inhibitor at final concentrations of 4 μM each). Cells were added (50p/well) to compound plates and the infected cells were maintained at 37° C./5% CO₂/90% relative humidity.

Infected cells were quantified at two time points, ˜48 h and ˜72 h post-infection, by counting the number of green cells in each well using an Acumen eX3 scanner. The increase in the number of green cells over ˜24 h period gives the reproductive ratio, R0, which is typically 5-15 and has been shown experimentally to be in logarithmic phase (data not shown). Inhibition of R0 is calculated for each well, and IC₅₀s determined by non-linear 4-parameter curve fitting. Assay IC₅₀ results are shown in the table below.

| | WILD TYPE | WILD TYPE |
|---|---|---|
| | CELL ASSAY | CELL ASSAY |
| | Viking IP | Viking IP |
| Compound | (0% NHS) | (100% NHS) |
| No. | (nM) | (nM) |
| 1 | 7.6 | 236.6 |
| 2 | 6.3 | 94.9 |
| 3 | 7.1 | 122 |
| 4 | 4.8 | 58.8 |
| 5 | 3.4 | 1405 |
| 6 | 4.7 | 303.3 |
| 7 | 2.7 | 651.9 |
| 8 | 2.3 | 96.1 |
| 9 | 2.1 | >42020 |
| 10 | 2.1 | 907.6 |
| 11 | 1.5 | 1328 |
| 12 | 3.5 | 215.1 |
| 13 | 4.0 | 705.5 |
| 14 | 4.9 | 112.5 |
| 15 | 4.4 | 164.6 |
| 16 | 3.3 | 64.7 |
| 17 | 2.5 | 968.9 |
| 18 | 1.7 | 155.4 |
| 19 | 1.7 | 45.0 |
| 20 | 1.4 | 215.9 |
| 21 | 3.7 | 927.2 |
| 22 | 3.0 | 227.3 |
| 23 | 1.9 | 121 |
| 24 | 1.2 | 201.4 |
| 25 | 1.4 | 1701 |
| 26 | 2.1 | 107.2 |
| 27 | 2.1 | 29 |
| 28 | 1.0 | 128.6 |
| 29 | 1.9 | 3127 |
| 30 | 2.1 | 410.2 |
| 31 | 1.7 | 268.4 |
| 32 | 2.1 | 55.7 |
| 33 | 3.0 | 1205 |
| 34 | 2.1 | 178.3 |
| 35 | 2.1 | 115.8 |
| 36 | 2.1 | 59.9 |
| 37 | 2.2 | 547.4 |
| 38 | 1.2 | 32.5 |
| 39 | 2.1 | 322.1 |
| 40 | 1.9 | 39.1 |
| 41 | 2.2 | 30.4 |
| 42 | 2.8 | 59.3 |
| 43 | 3.7 | 1269 |
| 44 | 3.7 | 259.4 |
| 45 | 1.1 | 17.9 |
| 46 | 1.6 | 36.8 |
| 47 | 2.3 | 1460 |
| 48 | 2.1 | 260.9 |
| 49 | 2.6 | 1018 |
| 50 | 2.9 | 25.5 |
| 51 | 1.7 | 32.2 |
| 52 | 2.7 | 149.7 |
| 53 | 2.6 | 4369 |
| 54 | 2.8 | 29.5 |
| 55 | 2.2 | 93.6 |
| 56 | 3.4 | 890.8 |
| 57 | 3.1 | 9974 |
| 58 | 2.3 | 270 |
| 59 | 3.2 | 105.6 |
| 60 | 7.1 | 1264 |
| 61 | 2.3 | 95.9 |
| 62 | 2.4 | 60.54 |
| 63 | 2.9 | 9297 |
| 64 | 2.9 | 2744 |
| 65 | 1.7 | 107.3 |
| 66 | 1.4 | 32.3 |
| 67 | 2.3 | 82.8 |
| 68 | 2.4 | 45.5 |
| 69 | 1.9 | 77.5 |
| 70 | 1.2 | 26.3 |
| 71 | 3.1 | 91.1 |
| 72 | 1.3 | 15.8 |
| 73 | 2.3 | 387 |
| 74 | 1.6 | 38.4 |
| 75 | 2.5 | 171.6 |
| 76 | 2.5 | 236.9 |
| 77 | 3.3 | 4472 |
| 78 | 3.2 | 383 |
| 79 | 4.5 | 942.2 |
| 80 | 2.8 | 192.3 |

### Treatment or Prevention of HIV Infection

The Tricyclic Heterocycle Compounds may be useful in the inhibition of HIV, the inhibition of HIV integrase, the treatment of HIV infection and/or reduction of the likelihood or severity of symptoms of HIV infection and the inhibition of HIV viral replication and/or HIV viral production in a cell-based system. For example, the Tricyclic Heterocycle Compounds may be useful in treating infection by HIV after suspected past exposure to HIV by such means as blood transfusion, exchange of body fluids, bites, accidental needle stick, or exposure to subject blood during surgery or other medical procedures.

Accordingly, in one embodiment, the invention provides methods for treating HIV infection in a subject, the methods comprising administering to the subject an effective amount of at least one Tricyclic Heterocycle Compound or a pharmaceutically acceptable salt or prodrug thereof. In a specific embodiment, the amount administered is effective to treat or prevent infection by HIV in the subject. In another specific embodiment, the amount administered is effective to inhibit HIV viral replication and/or viral production in the subject. In one embodiment, the HIV infection has progressed to AIDS.

The Tricyclic Heterocycle Compounds are also useful in the preparation and execution of screening assays for antiviral compounds. For example the Tricyclic Heterocycle Compounds may be useful for identifying resistant HIV cell lines harboring mutations, which are excellent screening tools for more powerful antiviral compounds. Furthermore, the Tricyclic Heterocycle Compounds may be useful in establishing or determining the binding site of other antivirals to the HIV Integrase.

The compositions and combinations of the present invention may be useful for treating a subject suffering from infection related to any HIV genotype.

### Combination Therapy

In another embodiment, the present methods for treating or preventing HIV infection can further comprise the administration of one or more additional therapeutic agents which are not Tricyclic Heterocycle Compounds.

In one embodiment, the additional therapeutic agent is an antiviral agent.

In another embodiment, the additional therapeutic agent is an immunomodulatory agent, such as an immunosuppressive agent.

Accordingly, in one embodiment, the present invention provides methods for treating a viral infection in a subject, the method comprising administering to the subject: (i) at least one Tricyclic Heterocycle Compound (which may include two or more different Tricyclic Heterocycle Compounds), or a pharmaceutically acceptable salt or prodrug thereof, and (ii) at least one additional therapeutic agent that is other than a Tricyclic Heterocycle Compound, wherein the amounts administered are together effective to treat or prevent a viral infection.

When administering a combination therapy of the invention to a subject, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a Tricyclic Heterocycle Compound and an additional therapeutic agent may be present in fixed amounts (dosage amounts) in a single dosage unit (e.g., a capsule, a tablet and the like).

In one embodiment, at least one Tricyclic Heterocycle Compound is administered during a time when the additional therapeutic agent(s) exert their prophylactic or therapeutic effect, or vice versa.

In another embodiment, at least one Tricyclic Heterocycle Compound and the additional therapeutic agent(s) are administered in doses commonly employed when such agents are used as monotherapy for treating a viral infection.

In another embodiment, at least one Tricyclic Heterocycle Compound and the additional therapeutic agent(s) are administered in doses lower than the doses commonly employed when such agents are used as monotherapy for treating a viral infection.

In still another embodiment, at least one Tricyclic Heterocycle Compound and the additional therapeutic agent(s) act synergistically and are administered in doses lower than the doses commonly employed when such agents are used as monotherapy for treating a viral infection.

In one embodiment, at least one Tricyclic Heterocycle Compound and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Viral infections and virus-related disorders that may be treated or prevented using the combination therapy methods of the present invention include, but are not limited to, those listed above.

In one embodiment, the viral infection is HIV infection.

In another embodiment, the viral infection is AIDS.

The at least one Tricyclic Heterocycle Compound and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of at least one Tricyclic Heterocycle Compound and the additional therapeutic agent(s) may inhibit the resistance of a viral infection to these agents.

As noted above, the present invention is also directed to use of a compound of Formula I with one or more anti-HIV agents. An “anti-HIV agent” is any agent which is directly or indirectly effective in the inhibition of HIV reverse transcriptase or another enzyme required for HIV replication or infection, the treatment or prophylaxis of HIV infection, and/or the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith. For example, the compounds of this invention may be effectively administered, whether at periods of pre-exposure and/or post-exposure, in combination with effective amounts of one or more anti-HIV agents selected from HIV antiviral agents, immunomodulators, antiinfectives, or vaccines useful for treating HIV infection or AIDS. Suitable HIV antivirals for use in combination with the compounds of the present invention include, for example, those listed in Table A as follows:

TABLE A

| Name | Type |
|---|---|
| abacavir, ABC, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Reyataz ® | PI |
| AZT, zidovudine, azidothymidine, Retrovir ® | nRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, DLV, Rescriptor ® | nnRTI |
| dolutegravir, Tivicay ® | II |
| Doravirine | nnRTI |
| efavirenz, EFV, Sustiva ®, Stocrin ® | nnRTI |
| efavirenz + emtricitabine + tenofovir DF, Atripla ® | nnRTI + nRTI |
| EFdA (4′-ethynyl-2-fluoro-2′-deoxyadenosine) | nRTI |
| emtricitabine, FTC, Emtriva ® | nRTI |
| emtricitabine + tenofovir DF, Truvada ® | nRTI |
| emvirine, Coactinon ® | nnRTI |
| enfuvirtide, Fuzeon ® | FI |
| enteric coated didanosine, Videx EC ® | nRTI |
| etravirine, TMC-125 | nnRTI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| Lopinavir | PI |
| lopinavir + ritonavir, Kaletra ® | PI |
| maraviroc, Selzentry ® | EI |
| nelfinavir, Viracept ® | PI |
| nevirapine, NVP, Viramune ® | nnRTI |
| rilpivirine, TMC-278 | nnRTI |
| ritonavir, Norvir ® | PI |
| saquinavir, Invirase ®, Fortovase ® | PI |
| stavudine, d4T, didehydrodeoxythymidine, Zerit ® | nRTI |
| tenofovir DF (DF = disoproxil fumarate), TDF, Viread ® | nRTI |
| tipranavir, Aptivus ® | PI |

EI = entry inhibitor; FI = fusion inhibitor; PI = protease inhibitor; nRTI = nucleoside reverse transcriptase inhibitor; II =integrase inhibitor; nnRTI = non-nucleoside reverse transcriptase inhibitor. Some of the drugs listed in the table are used in a salt form; e.g., abacavir sulfate, indinavir sulfate, atazanavir sulfate, nelfinavir mesylate.

In one embodiment, one or more anti-HIV drugs are selected from, lamivudine, abacavir, ritonavir, darunavir, atazanavir, emtricitabine, tenofovir, rilpivirine and lopinavir.

In another embodiment, the compound of formula (I) is used in combination with lamivudine.

In still another embodiment, the compound of formula (I) is used in combination atazanavir.

In another embodiment, the compound of formula (I) is used in combination with darunavir.

In another embodiment, the compound of formula (I) is used in combination with rilpivirine.

In one embodiment, the compound of formula (I) is used in combination with lamivudine and abacavir.

In another embodiment, the compound of formula (I) is used in combination with darunavir.

In another embodiment, the compound of formula (I) is used in combination with emtricitabine and tenofovir.

In still another embodiment, the compound of formula (I) is used in combination atazanavir.

In another embodiment, the compound of formula (I) is used in combination with ritonavir and lopinavir.

In one embodiment, the compound of formula (I) is used in combination with abacavir and lamivudine.

In another embodiment, the compound of formula (I) is used in combination with lopinavir and ritonavir.

In one embodiment, the present invention provides pharmaceutical compositions comprising (i) a compound of formula (I) or a pharmaceutically acceptable salt or prodrug thereof; (ii) a pharmaceutically acceptable carrier; and (iii) one or more additional anti-HIV agents selected from lamivudine, abacavir, ritonavir and lopinavir, or a pharmaceutically acceptable salt or prodrug thereof, wherein the amounts present of components (i) and (iii) are together effective for the treatment or prophylaxis of infection by HIV or for the treatment, prophylaxis, or delay in the onset or progression of AIDS in the subject in need thereof.

In another embodiment, the present invention provides a method for the treatment or prophylaxis of infection by HIV or for the treatment, prophylaxis, or delay in the onset or progression of AIDS in a subject in need thereof, which comprises administering to the subject (i) a compound of formula (I) or a pharmaceutically acceptable salt or prodrug thereof and (ii) one or more additional anti-HIV agents selected from lamivudine, abacavir, ritonavir and lopinavir, or a pharmaceutically acceptable salt or prodrug thereof, wherein the amounts administered of components (i) and (ii) are together effective for the treatment or prophylaxis of infection by HIV or for the treatment, prophylaxis, or delay in the onset or progression of AIDS in the subject in need thereof.

It is understood that the scope of combinations of the compounds of this invention with anti-HIV agents is not limited to the HIV antivirals listed in Table A, but includes in principle any combination with any pharmaceutical composition useful for the treatment or prophylaxis of AIDS. The HIV antiviral agents and other agents will typically be employed in these combinations in their conventional dosage ranges and regimens as reported in the art, including, for example, the dosages described in the Physicians' Desk Reference, Thomson PDR, Thomson PDR, 57^{th} edition (2003), the 58^{th} edition (2004), the 59^{th} edition (2005), and the like. The dosage ranges for a compound of the invention in these combinations are the same as those set forth above.

The doses and dosage regimen of the other agents used in the combination therapies of the present invention for the treatment or prevention of HIV infection may be determined by the attending clinician, taking into consideration the approved doses and dosage regimen in the package insert; the age, sex and general health of the subject; and the type and severity of the viral infection or related disease or disorder. When administered in combination, the Tricyclic Heterocycle Compound(s) and the other agent(s) may be administered simultaneously (i.e., in the same composition or in separate compositions one right after the other) or sequentially. This is particularly useful when the components of the combination are given on different dosing schedules, e.g., one component is administered once daily and another component is administered every six hours, or when the pharmaceutical compositions are different, e.g., one is a tablet and one is a capsule. A kit comprising the separate dosage forms is therefore advantageous.

### Compositions and Administration

When administered to a subject, the Tricyclic Heterocycle Compounds may be administered as a component of a composition that comprises a pharmaceutically acceptable carrier or vehicle. The present invention provides pharmaceutical compositions comprising an effective amount of at least one Tricyclic Heterocycle Compound and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules may be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Additionally, the compositions of the present invention may be formulated in sustained release form to provide the rate controlled release of any one or more of the components or active ingredients to optimize therapeutic effects, i.e., antiviral activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the one or more Tricyclic Heterocycle Compounds are administered orally.

In another embodiment, the one or more Tricyclic Heterocycle Compounds are administered intravenously.

In one embodiment, a pharmaceutical preparation comprising at least one Tricyclic Heterocycle Compound is in unit dosage form. In such form, the preparation is subdivided into unit doses containing effective amounts of the active components.

Compositions may be prepared according to conventional mixing, granulating or coating methods, respectively, and the present compositions can contain, in one embodiment, from about 0.1% to about 99% of the Tricyclic Heterocycle Compound(s) by weight or volume. In various embodiments, the present compositions can contain, in one embodiment, from about 1% to about 70% or from about 5% to about 60% of the Tricyclic Heterocycle Compound(s) by weight or volume.

The compounds of Formula I may be administered orally in a dosage range of 0.001 to 1000 mg/kg of mammal (e.g., human) body weight per day in a single dose or in divided doses. One dosage range is 0.01 to 500 mg/kg body weight per day orally in a single dose or in divided doses. Another dosage range is 0.1 to 100 mg/kg body weight per day orally in single or divided doses. For oral administration, the compositions may be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the subject to be treated. The specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24 hour period. In still another embodiment, the total daily dosage is administered in four divided doses over a 24 hour period.

The unit dosages of the Tricyclic Heterocycle Compounds may be administered at varying frequencies. In one embodiment, a unit dosage of a Tricyclic Heterocycle Compound may be administered once daily. In another embodiment, a unit dosage of a Tricyclic Heterocycle Compound may be administered twice weekly. In another embodiment, a unit dosage of a Tricyclic Heterocycle Compound may be administered once weekly. In still another embodiment, a unit dosage of a Tricyclic Heterocycle Compound may be administered once biweekly. In another embodiment, a unit dosage of a Tricyclic Heterocycle Compound may be administered once monthly. In yet another embodiment, a unit dosage of a Tricyclic Heterocycle Compound may be administered once bimonthly. In another embodiment, a unit dosage of a Tricyclic Heterocycle Compound may be administered once every 3 months. In a further embodiment, a unit dosage of a Tricyclic Heterocycle Compound may be administered once every 6 months. In another embodiment, a unit dosage of a Tricyclic Heterocycle Compound may be administered once yearly.

The amount and frequency of administration of the Tricyclic Heterocycle Compounds will be regulated according to the judgment of the attending clinician considering such factors as age, condition and size of the subject as well as severity of the symptoms being treated. The compositions of the invention can further comprise one or more additional therapeutic agents, selected from those listed above herein.

### Kits

In one aspect, the present invention provides a kit comprising a therapeutically effective amount of at least one Tricyclic Heterocycle Compound, or a pharmaceutically acceptable salt or prodrug of said compound and a pharmaceutically acceptable carrier, vehicle or diluent.

In another aspect the present invention provides a kit comprising an amount of at least one Tricyclic Heterocycle Compound, or a pharmaceutically acceptable salt or prodrug of said compound and an amount of at least one additional therapeutic agent listed above, wherein the amounts of the two or more active ingredients result in a desired therapeutic effect. In one embodiment, the one or more Tricyclic Heterocycle Compounds and the one or more additional therapeutic agents are provided in the same container. In one embodiment, the one or more Tricyclic Heterocycle Compounds and the one or more additional therapeutic agents are provided in separate containers.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1. A compound of the formula: 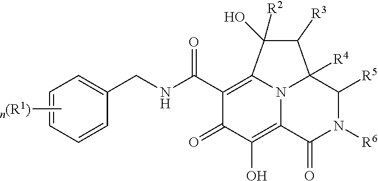
or a pharmaceutically acceptable salt thereof,
wherein:
each occurrence of R¹ is independently halo, hydroxyl, C₁₋₆ alkyl and —O—(C₁-C₆ alkyl);
R² is hydrogen, methyl or ethyl;
R³ is hydrogen, methyl or ethyl;
R⁴ is C₁₋₆ alkyl or (C₁₋₆ alkyl)OR⁷;
R⁵ is hydrogen, C₁₋₆ alkyl or (C₁₋₆ alkyl)OR⁷;
R⁶ is hydrogen, C₁₋₆ alkyl or (C₁₋₆ alkyl)OR⁷;
R⁷ is hydrogen or C₁₋₆ alkyl, which is optionally substituted with one to three halo;
n is an integer between one and three.

2. The compound of claim 1 wherein each R¹ is halo, or a pharmaceutically acceptable salt thereof.

3. The compound of claim 1 wherein R² is hydrogen or methyl, or a pharmaceutically acceptable salt thereof.

4. The compound of claim 1 wherein R³ is hydrogen or methyl, or a pharmaceutically acceptable salt thereof.

5. The compound of claim 1 wherein R⁴ is methyl, ethyl, CH₂OCH₃, CH₂CH₂OCH₃, CH₂CH₂OCHF₂, or a pharmaceutically acceptable salt thereof.

6. The compound of claim 1 wherein R⁴ is methyl or ethyl, or a pharmaceutically acceptable salt thereof.

7. The compound of claim 1 wherein R⁵ is hydrogen or methyl, or a pharmaceutically acceptable salt thereof.

8. The compound of claim 1 wherein R⁶ is methyl or ethyl, or a pharmaceutically acceptable salt thereof.

9. The compound of claim 1 selected from: 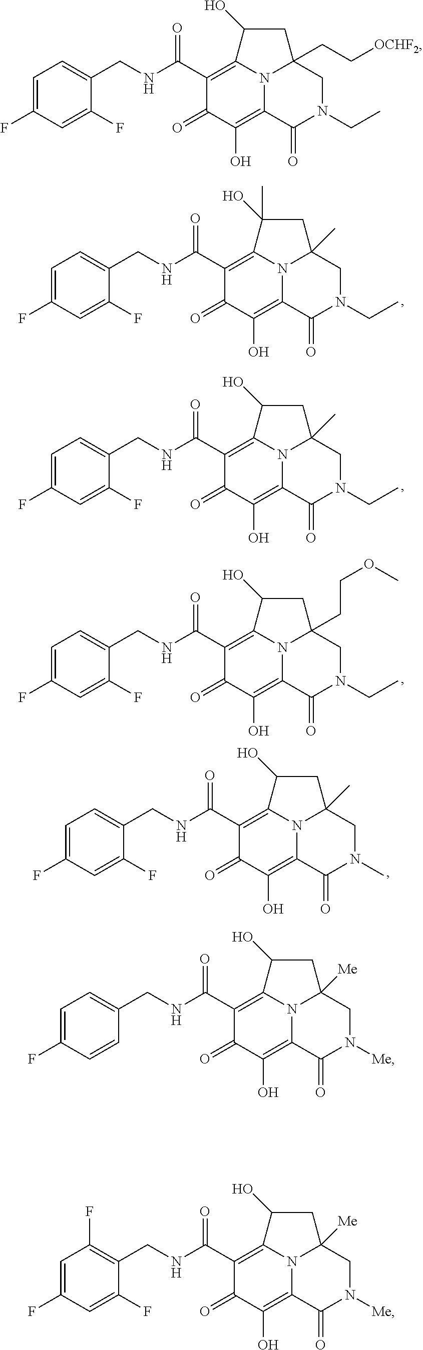 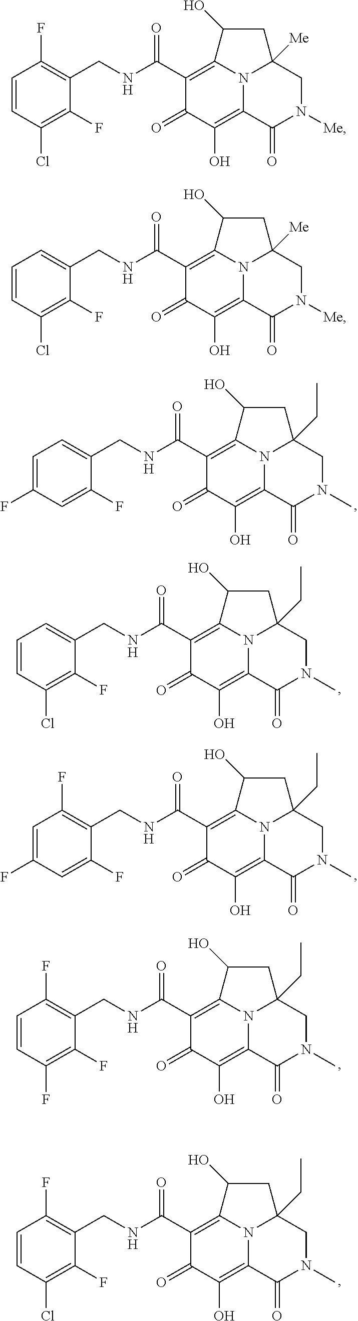 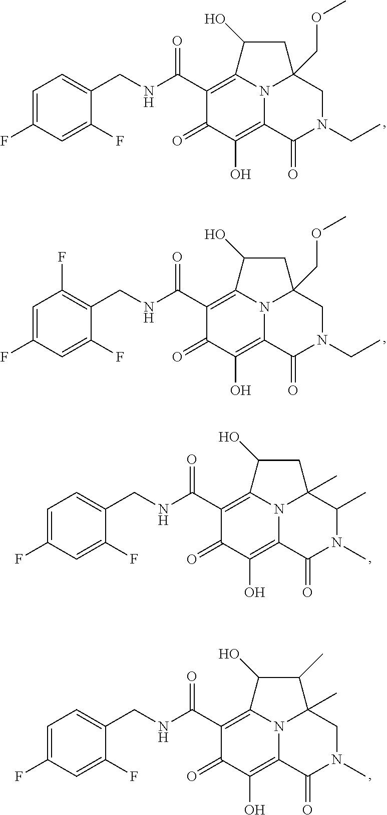
or a pharmaceutically acceptable salt thereof.

10. A pharmaceutical composition comprising an effective amount of a compound according to claim 1, or a pharmaceutically acceptable salt thereof, and a pharmaceutically acceptable carrier.

11. A method for the inhibition of HIV integrase in a subject in need thereof which comprises administering to the subject an effective amount of the compound according to claim 1, or a pharmaceutically acceptable salt thereof.

12. A method for the treatment of infection by HIV or for the treatment of AIDS in a subject in need thereof, which comprises administering to the subject an effective amount of the compound according to claim 1, or a pharmaceutically acceptable salt thereof.

13. The pharmaceutical composition of claim 10, further comprising one or more additional therapeutic agents selected from, raltegravir, lamivudine, abacavir, ritonavir, dolutegravir, arunavir, atazanavir, emtricitabine, tenofovir, elvitegravir, rilpivirine and lopinavir.

14. The method of claim 12, further comprising administering to the subject one or more additional therapeutic agents selected from raltegravir, lamivudine, abacavir, ritonavir, dolutegravir, arunavir, atazanavir, emtricitabine, tenofovir, elvitegravir, rilpivirine and lopinavir, wherein the amounts administered of the compound of claim 1 and the one or more additional therapeutic agents, are together effective to treat infection by HIV or to treat, prevent or delay the onset or progression of AIDS.

15. (canceled)

16. (canceled)
